SEQUENCE LISTING (20 sequences)

SEQ ID NO: 1 - protein, 21 aa, Artificial
  Synthetic Construct; linker between luciferase and Mdm2
GSGGGSGGGGSGGGGSGGTGS

SEQ ID NO: 2 - DNA, 48 bp, Artificial
  Synthetic Construct
ggaggcggaagtggtggaggcggaagcggcggtggaggtagtggtgga

SEQ ID NO: 3 - DNA, 54 bp, Artificial
  Synthetic Construct
gctactgctagccaccatggaagacgccaaaaacataaagaaaggcccggcgcc

SEQ ID NO: 4 - DNA, 46 bp, Artificial
  Synthetic Construct
agtagcagtagcggatcccacggcgatctttccgcccttcttggcc

SEQ ID NO: 5 - DNA, 123 bp, Artificial
  Synthetic Construct
gctactgctactggatccggaggcggaagtggtggaggcggaagcggcggtggaggtagtggtggaaccggttgcaataccaacatgtctgtacctactgatggtgctgtaaccacctcacag

SEQ ID NO: 6 - DNA, 48 bp, Artificial
  Synthetic Construct
gctactctcgagctaggggaaataagttagcacaatcatttgaattgg

SEQ ID NO: 7 - DNA, 20 bp, Artificial
  Synthetic Construct
ttggcgtgccaagcttctct

SEQ ID NO: 8 - DNA, 20 bp, Artificial
  Synthetic Construct
tacctgagtccgatgattcc

SEQ ID NO: 9 - DNA, 24 bp, Artificial
  Synthetic Construct
ggcggcagaccagcatgacagatt

SEQ ID NO: 10 - DNA, 20 bp, Artificial
  Synthetic Construct
gcagggggcggccagggtat

SEQ ID NO: 11 - DNA, 22 bp, Artificial
  Synthetic Construct
cctggagggtcctgtacaatct

SEQ ID NO: 12 - DNA, 21 bp, Artificial
  Synthetic Construct
gcacctaattgggctccatct

SEQ ID NO: 13 - DNA, 21 bp, Artificial
  Synthetic Construct
tggagctgcagaggatgattg

SEQ ID NO: 14 - DNA, 21 bp, Artificial
  Synthetic Construct
aaacatgtcagctgccactcg

SEQ ID NO: 15 - DNA, 21 bp, Artificial
  Synthetic Construct
tgacactggcaaaacaatgca

SEQ ID NO: 16 - DNA, 21 bp, Artificial
  Synthetic Construct
ggtccttttcaccagcaagct

SEQ ID NO: 17 - DNA, 66 bp, Artificial
  Synthetic Construct
gcggaccgatgtatccatatgacgtcccagactatgccacatcattttccacctctgctcagtgtt

SEQ ID NO: 18 - DNA, 28 bp, Artificial
  Synthetic Construct
ggggtaccttatgctataaaaaccttaa

SEQ ID NO: 19 - DNA, 21 bp, Artificial
  Synthetic Construct
aacttacgctgagtacttcga

SEQ ID NO: 20 - DNA, 21 bp, Artificial
  Synthetic Construct
agagattcagctggttattaa

(19) United States
(12) Patent Application Publication
(10) Pub. No.: US 2012/0135089 A1
(43) Pub. Date: May 31, 2012

(54) E3 LIGASE INHIBITORS

(76) Inventors: Brent R. Stockwell, New York, NY (US); Ariel Herman, New York, NY (US)

(21) Appl. No.: 13/256,680

(22) PCT Filed: Mar. 17, 2010
(86) PCT No.: PCT/US10/00793

§ 371 Date: Jan. 9, 2012

Related U.S. Application Data
(60) Provisional application No. 61/210,322, filed on Mar. 17, 2009.
(60) Provisional application No. 61/274,901, filed on Aug. 20, 2009.

(51) Int. Cl. A61K 33/24; A61K 31/515; C12N 9/99; C07D 471/04; A61K 31/573; A61K 31/5377; A61K 38/12; A61K 31/704; A61K 31/675; A61K 31/7048; C07D 403/06; A61K 31/437

(52) U.S. Cl. 424/649; 544/300; 514/270; 435/184; 546/87; 514/292; 514/274; 544/123; 514/232.8; 514/19.9; 514/34; 514/110; 514/27; 514/83; 514/171

(57) ABSTRACT

The present invention provides, e.g., compounds having the structure of formula (I): The present invention also provides pharmaceutical compositions that include such compounds, and methods for modulating murine double minute 2 protein (Mdm2) E3 ligase activity, particularly the Mdm2-MdmX hetero-complex E3 ligase activity, using such compounds.

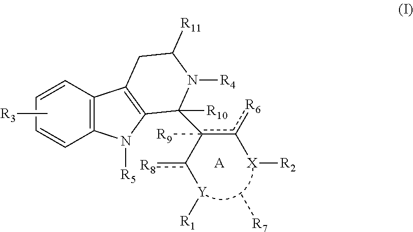

### CROSS-REFERENCE TO RELATED APPLICATIONS

The present application claims benefit to U.S. provisional application Ser. No. 61/210,322 filed Mar. 17, 2009 and U.S. provisional application Ser. No. 61/274,901 filed Aug. 20, 2009. The entire contents of both applications are incorporated by reference herein.

### FEDERALLY SPONSORED RESEARCH OR DEVELOPMENT

This invention was made with government support under Grant number R03MH082369-01 awarded by the National Institutes of Health. The government has certain rights in the invention.

### FIELD OF THE INVENTION

The present invention relates to, inter alia, compounds, pharmaceutical compositions, and methods for modulating E3 ligase activity, particularly murine double minute 2 protein Mdm2 (Mdm2)-MdmX hetero-complex E3 ligase activity.

### BACKGROUND OF THE INVENTION

The ubiquitin-dependent proteasome pathway is the main cellular mechanism for controlling the degradation of proteins in diverse cellular pathways. The ubiquitin pathway regulates key components of cell physiology, and therefore mutations in the pathway underlie many human diseases, including cancer. There are three steps in ubiquitination: (i) activation of a ubiquitin activating enzyme (E1) by the addition of a 76-amino acid ubiquitin moiety, (ii) transfer of a ubiquitin moiety to a cysteine residue on a ubiquitin conjugating enzyme (E2) and (iii) formation of an isopeptide bond between the ubiquitin moiety and a lysine in the target protein, catalyzed by a ubiquitin ligase (E3) (1). The specificity of the process is controlled by the E3 enzyme, which recognizes and interacts with the target protein to be degraded. One class of E3s is the RING domain proteins that mediate the transfer of ubiquitin from the E2 to the E3-bound substrate.

In humans, there are two E1s, 36 E2s and up to 1000 E3s (20). Two classes of E3s are HECT and RING domains. The RING domain proteins can be further subdivided into the canonical RING domain and the RING-related domains known as U box and PHD finger (21). While HECT domain ligases contain a 350-residue ligase domain that directly binds the activated ubiquitin molecule (22, 23), RING finger ligases serve as a scaffold for spatially coordinating the ubiquitin source and the target molecule (24). Although the RING finger motifs show little homology to each other (25), the core conserved structure for these domains is a pattern of cysteine and histidine residues coordinating two zinc ions (26).

For many protein classes, no small molecule ligands exist (27, 28). Accordingly, developing inhibitors of these protein classes represents an important challenge. By discovering small molecule modulators of these targets, the “druggable genome”, that is, the subset of disease-modifying proteins encoded by the human genome that can be targeted with small molecule therapeutics, will be expanded. The number of proteins targeted by existing small molecule drugs has been estimated to be just 483 in one study (31) and 399 in another study (27). Moreover, half of all existing drugs target just five protein families (G-protein-coupled receptors, protein kinases, proteases, nuclear hormone receptors and phosphodiesterases) (27). Additional druggable domains include ion channels, transporters, phosphatases, cytochrome P450s and metabolic enzymes. Based on analysis of the human genome sequence, the total number of potentially druggable targets in these classes may be as small as 2,200, given one recent analysis (28). Since there are up to 1000 E3 ligases encoded in the human genome, a strategy for inhibiting E3 ligases with small molecules would significantly expand the druggable protein universe.

The murine double minute 2 protein (Mdm2, also used to denote the human protein), is an E3 ligase of paramount importance in cancer biology because of its ability to regulate the tumor suppressor protein, p53. p53 is a transcription factor that controls a large number of genes involved in apoptosis, growth arrest, and senescence. Although there are other known E3 ligases for p53, Mdm2 is believed to be the ligase of primary importance for regulating p53. This was explicitly illustrated by the deletion of the Mdm2 gene which is embryonic lethal in mice, but can be rescued by the additional deletion of the p53 gene (2-3). In normal unstressed cells, p53 levels are kept low by the negative regulator Mdm2 via two primary mechanisms. First, Mdm2 causes the rapid degradation of p53 through ubiquitination and proteasomal degradation (4-6) and, secondly, Mdm2 binds and inactivates the N-terminal transactivation domain of p53, preventing transcriptional activation (7). Upon stress signals, such as DNA-damage or expression of oncogenes, post-translational modifications of both p53 and Mdm2 occur that prevent their ability to interact, stabilizing p53. This allows p53 to accumulate and function as a transcription factor. Additionally, p53 activates the transcription of Mdm2, creating an auto-regulatory negative feedback loop between Mdm2 and p53. Once the stress response is over, p53 levels return to basal levels, due to Mdm2 regulation. Additionally, Mdm2 also has ubiquitin ligase activity against itself, therefore regulating its own expression (8). Although there are other known E3 ligases targeting p53, Mdm2 is believed to be of primary importance in p53 regulation. This was explicitly illustrated by the rescue of mouse embryonic lethality caused by the deletion of Mdm2, by a concomitant deletion of p53.

The Mdm2 homologue, MdmX (also called Mdm4 and used to denote both the human and mouse form of the protein), is a non-redundant and essential p53 regulator exemplified in mouse loss-of-function studies (48-50). Similarly to Mdm2, MdmX is overexpressed in human tumors that are generally distinct from p53-mutant tumors (51). Experimental evidence suggests that Mdm2 and MdmX function together to inhibit p53 activity. MdmX can interact with p53 and inhibit its transactivation (52). Although MdmX has no intrinsic RING activity on its own, Mdm2 and MdmX can form hetero-oligomers through their RING domains (53) whereby MdmX can augment Mdm2 activity (54, 55). Through this RING domain interaction, Mdm2 can also directly ubiquitinate and degrade MdmX upon DNA-damage stimuli (56-58).

The importance of the p53 pathway in tumor progression is revealed by the fact that approximately 50% of tumors have a mutation in the TP53 gene. Additionally, overexpression of Mdm2 occurs in approximately 7% of all human tumors and 20% of soft tissue tumors, which can lead to the inactivation of p53 and tumor progression, (9). In p53 wild-type tumors, there is the possibility of stimulating p53 by inhibiting the negative regulatory affects of Mdm2, thereby re-establishing p53 activity and inducing cell death. p53 activating signals can exist in established animal tumors and restoring p53 in animal models can inhibit tumor growth (10-11). Additionally, because Mdm2 has oncogenic effects independent of p53 (12), Mdm2 inhibitors may be useful in mutant or null p53 cancers, as well as those with wild-type p53.

Mdm2 has several conserved domains, including a p53-binding domain, a nuclear localization signal, a nuclear export signal, an acidic domain, a zinc finger domain and a RING domain that mediates the E3 ubiquitin ligase activity (29). The RING domain functions in concert with the E2 UbCH5 proteins (Ubc5a, b, or c) to induce ubiquitination of substrate proteins such as p53 (29). Mdm2 E3 ligase inhibitors could be used to define novel substrates and functions of Mdm2 that are independent of p53. Several low potency and moderate selectivity inhibitors of Mdm2 E3 ligase activity have been reported, and these compounds were reported to have p53-dependent lethality in tumor cells (30).

Inhibitors of the Mdm2-UBCH5 interaction should disrupt the E3 ligase activity of Mdm2 and therefore its oncogenic activity. Such inhibitors could be developed into novel therapeutic agents for sarcomas involving Mdm2 overexpression or amplification, irrespective of their p53 status. Mdm2 E3 ligase inhibitors could be used to define novel substrates and functions of Mdm2 that are independent of p53.

Small molecules that inhibit Mdm2 may reduce the side effects of traditional chemotherapeutic agents, by potentiating their anti-tumor lethality. Small molecule inhibitors of the Mdm2-p53 protein-protein interaction, most notably the nutlins, have been shown to activate p53, thereby killing tumor cells with wild-type p53 (13). However, the nutlins, and other such compounds, disrupt the N-terminal region of Mdm2 binding to p53 and have no effect on the E3 ligase of Mdm2; such compounds are not likely to inhibit p53-independent functions of Mdm2. More recently, the HLI series of compounds (HL198 and HL1373) as well as other compounds that have not been fully characterized, all discovered in in vitro ubiquitination assays have shown inhibition of Mdm2 E3 ligase activity (14-19). These compounds revealed that inhibiting Mdm2 has therapeutic potential for re-activating p53. However, the Nutlins do not inhibit Mdm2 E3 ligase activity and so do not block p53-independent functions of Mdm2 and the HLI compounds have non-specific effects at higher concentrations. For these reasons, there is a pressing need for more specific and novel inhibitors of Mdm2 E3 ligase activity.

### SUMMARY OF THE INVENTION

To identify Mdm2 E3 ligase inhibitors that might act through new mechanisms, a novel high-throughput cell-based auto-ubiquitination assay used to identify inhibitors of Mdm2 E3 ligase activity has been developed. This is the first cell-based screen used to identify inhibitors of the E3 ligase activity of Mdm2, and can be readily adapted in order to identify inhibitors of other E3 ligases. With this assay, two related compounds were discovered that inhibit Mdm2 E3 ligase activity, in particular, Mdm2-MdmX hetero-complex E3 ligase activity, which were named Mdm2 E3 Ligase inhibitors 23 and 24 (MEL23 and MEL24). This class of compounds has a tetrahydro-beta-carboline and barbituric acid scaffold. Treatment of multiple cell lines with MEL23 and MEL24 inhibited Mdm2 and p53 ubiquitin conjugates and increased the expression of Mdm2 and p53. MEL compounds decreased cell survival in a p53-dependent manner and increased sensitivity to DNA-damaging agents. It is to be understood that reference in the present application to “MEL compounds” and like phrases means MEL23 and/or MEL24 (and not MEL3, 4, or 17) unless specifically, or by context, indicated otherwise.

Most tumors inactivate the p53 pathway. For this reason, small molecules that can re-activate p53 may be useful in the clinic. It is necessary to both increase p53 expression and increase p53 activity. Because Mdm2 can inhibit p53 through two independent mechanisms—E3 ligase-mediated degradation and binding—it was hypothesized that inhibitors of Mdm2 E3 ligase activity alone may not be sufficient to activate p53. However, the applicants have shown that inhibiting Mdm2 E3 ligase activity, particularly Mdm2-MdmX hetero-complex E3 ligase activity, alone is sufficient to cause p53-dependent cell death. Many tumor cells already have p53 activating signals in place, rendering further p53 activation unnecessary.

The additional benefit of the MEL compounds and other specific Mdm2 E3 ligase inhibitors is that they may be beneficial in p53-null or p53-mutant tumors. Mdm2 has been shown to have p53-independent oncogenic activity. It is possible that inhibiting the E3 ligase activity of Mdm2 may lead to inhibition of some of its oncogenic activity.

In addition to the potential use of the MEL compounds as anti-tumor agents, they may have utility in increasing longevity. For example, it has been demonstrated in murine models that an extra copy of p53 and Arf, which is a positive regulator of p53, causes an increase in median lifespan (33). This indicates that compounds that increase p53 levels without affecting regulation of the protein or gene may be useful for increasing longevity. Because the MEL compounds increase p53 levels without activating p53, these compounds may be an unexpectedly efficient means of causing p53-dependent increases in longevity.

In view of the foregoing, the following embodiments are provided to exemplify the present invention. One embodiment of the present invention is a compound according to formula (I):

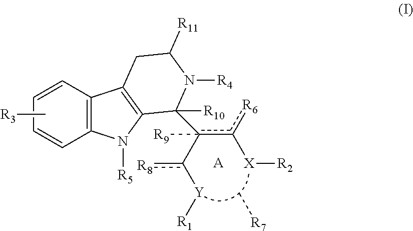

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, (2) R₄ is not methyl, and (3) R₃ is not methoxy;
   and with the third proviso that the compound is not:

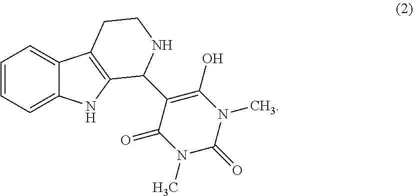

Another embodiment of the present invention is a pharmaceutical composition. This pharmaceutical composition comprises a pharmaceutically acceptable carrier and a compound according to formula (I):

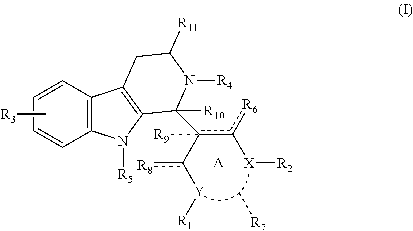

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, (2) R₄ is not methyl, and (3) R₃ is not methoxy;
   and with the third proviso that the compound is not:

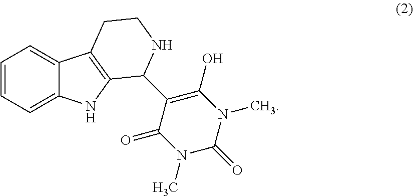

Another embodiment of the present invention is a pharmaceutical composition. This pharmaceutical composition comprises a compound according to the present invention, a pharmaceutically acceptable carrier, and at least one DNA damaging agent.

Yet another embodiment of the present invention is a method for modulating murine double minute 2 protein (Mdm2) ligase activity. This method comprises administering to a cell an amount of a compound according to formula (I):

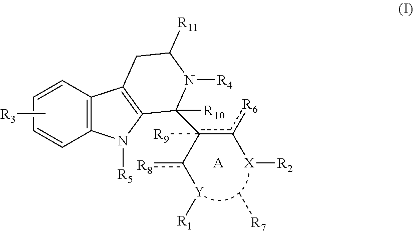

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing; if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, and (2) R₄ is not methyl;
   which amount is effective to modulate Mdm2 ligase activity.

A further embodiment of the present invention is a method for treating or ameliorating the effects of a cancerous disease. This method comprises administering to a patient in need thereof an amount of a compound according to formula (I):

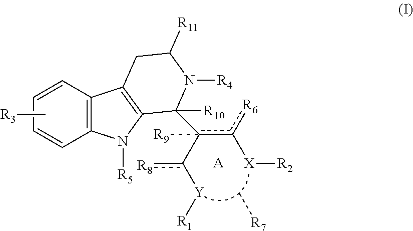

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, and (2) R₄ is not methyl;
   which amount is effective to treat or ameliorate the effects of the cancerous disease.

Another embodiment of the present invention is a compound. This compound has the structure of

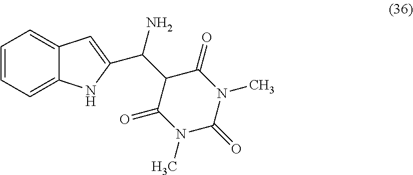

or an enantiomer, optical isomer, diastereomer, N-oxide, crystalline form, hydrate, or pharmaceutically acceptable salt thereof.

Yet another embodiment of the present invention is a pharmaceutical composition comprising the compound 36 disclosed above.

A further embodiment of the present invention is a method for modulating murine double minute 2 protein (Mdm2) ligase activity. This method comprises administering to a cell an amount of a compound 36 disclosed above, which amount is effective to modulate Mdm2 ligase activity.

Yet another embodiment of the present invention is a method for treating or ameliorating the effects of a cancerous disease. This method comprises administering to a patient in need thereof an amount of a compound 36 disclosed above, which amount is effective to treat or ameliorate the effects of the cancerous disease.

### BRIEF DESCRIPTION OF THE DRAWINGS

FIG. 1 depicts various experiments that verify a cell-based luminescence assay according to the present invention. FIG. 1A shows that the expression levels of Mdm2(C464A)-luciferase are higher than that of Mdm2-luciferase. 293T cells were transfected with no DNA, Mdm2-luciferase, or Mdm2(C464A)-luciferase. Forty-eight hours later, cells were lysed, and Mdm2 levels were analyzed by Western blotting with anti-Mdm2 and anti-actin antibodies. FIGS. 1B and 1C show that Mdm2-luciferase protein is regulated by proteasomes. Stable cell lines were seeded in 384-well plates and 24 hours later treated with MG132 for 2 hours or cycloheximide for 30 minutes at the indicated concentrations. Cells were lysed with a Steadylite luminescence buffer (PerkinElmer, Waltham, Mass.) and analyzed on a Victor3 Plate Reader. Each graph represents the median of 10 replicates normalized to a no treatment control; one standard deviation is shown. FIG. 1D shows that p53 levels are lower in Mdm2-luciferase cells than in Mdm2(C464A)-luciferase cells. 293T and stable cell lines were lysed, and equal amounts of cell extracts were analyzed by Western blotting with anti-p53 and anti-eIF4E antibodies. Relative levels of Mdm2 protein in stable cell lines can be seen in FIG. 1E, lanes 1 and 4. FIG. 1E shows that Mdm2-luciferase protein is ubiquitinated, whereas Mdm2(C464A)-luciferase protein is not. Stable cell lines were transfected with or without ubiquitin-HA. Forty-eight hours later, cells were lysed and immunoprecipitated with anti-HA beads, and immunoprecipitated samples were analyzed by Western blotting with an anti-Mdm2 antibody.

FIG. 2 shows the structures of certain of the MEL compounds and their effects on Mdm2, p53, and MDMX in cells. FIG. 2A shows the structures of hit compounds MEL23 and MEL24 and their activity in a cell-based auto-ubiquitination assay. Cells were plated in 384-well format and treated with MG132 (2 hours) or cycloheximide (30 minutes). Steadylite luminescence buffer (PerkinElmer) was added and the relative luminescence levels determined on a Victor3 Plate Reader. Each graph represents the median of 5 replicates normalized to a no treatment control, and one standard deviation is shown. The EC₅₀ for MEL23 and MEL24 are 2.7 μg/ml and 3.0 μg/ml (7.5 μM and 9.2 μM) respectively. The graphs and EC₅₀ values were determined using Graphpad PRISM®. FIG. 2B shows that Mdm2 and p53 levels are increased with MEL compound treatment. U2OS, RKO, and HCT116 cells (p53 wild-type) were incubated with 5 μg/ml (14 μM) of MEL23, 5 μg/ml (15 μM) of MEL24, or 10 μM of MG132 for 6 hours. Cells were lysed and analyzed by Western blotting using anti-Mdm2, anti-p53, and anti-eIF4E antibodies. NT indicates a no treatment control. FIG. 2C shows that MEL compounds inhibit etoposide-induced MdmX degradation. MCF7 cells (p53 wild-type) were incubated with 30 μg/ml (51 μM) etoposide alone or in combination with 50 μM AlIn or 5 μg/ml (14 μM) MEL23, or 5 μg/ml (15 μM) MEL24 for 16 hours. Cells were lysed and analyzed by Western blotting using anti-MDMX and anti-actin antibodies.

FIG. 3 shows that MEL23 and MEL24 are specific towards Mdm2 and cause Mdm2 accumulation independent of p53 transcription. FIG. 3A shows that MEL23 and MEL24 function independently of p53. H1229 cells were transfected with Mdm2-Flag or remained untransfected. Cells were treated with 5 μg/ml (14 μM) MEL23, 5 μg/ml (15 μM) MEL24, 10 μM MG132, or 1 μg/ml of doxorubicin for 6 hours. Cells were lysed and analyzed by Western blotting using anti-Mdm2 and anti-actin antibodies. The lower band indicated with a star seen with the Mdm2 antibody is a non-specific band. NT indicates a no treatment control. FIG. 3B shows that MEL23 and MEL24 increase p53 expression levels in RKO cells, but not RK0-E6 cells. RKO and RK0-E6 cells were treated with 5 μg/ml (14 μM) MEL23, 5 μg/ml (15 μM) MEL24, 10 μM MG132, 0.5 μg/ml doxorubicin, 0.5 μg/ml camptothecin or 25 μg/ml Etoposide for 6 hours. Cells were lysed and analyzed by Western blotting using anti-Mdm2, anti-p53, and anti-actin antibodies. The lower band indicated with a star seen with the anti-Mdm2 antibody is a non-specific band. FIG. 3C shows that MEL23 and MEL24 do not induce p53-Ser15 phosphorylation. RKO cells were treated with 5 μg/ml (14 μM) MEL23, 5 μg/ml (15 μM) MEL24, 10 μM MG132 or 2 μg/ml doxorubicin for 6 hours. Cells were lysed and analyzed by Western blotting using anti-Mdm2, anti-p53, anti-phospho-Ser15 p53, and anti-eiF4E antibodies.

FIG. 4 shows that MEL23 and MEL24 inhibit Mdm2 auto-ubiquitination and p53 ubiquitination in cell based assays. To obtain the results shown in FIG. 4A, Mdm2-luciferase cells were transfected with ubiquitin-HA and treated 8 hours later with 10 μg/ml (28 μM) MEL23, 10 μg/ml (31 μM) MEL24, or a DMSO control. After 16 hours, cells were additionally treated with 10 μM MG132 or a DMSO control. After 3 hours, cells were lysed and immunoprecipitated for 3 hours with anti-HA affinity matrix. Total lysates and immunoprecipitated samples were analyzed by Western blotting using anti-Mdm2 antibody. NT indicates a no treatment control. FIG. 4B shows a Western blot using anti-p53 antibodies in which H1299 cells were transfected with Mdm2-flag, p53, and Ubiquitin-HA. After 8 hours, cells were treated with 10 μg/ml (28 μM) MEL23, 10 μg/ml (31 μM) MEL24, or a DMSO control. After 16 hours, cells were additionally treated with 10 μM MG132 or a DMSO control. After 3 hours cells were lysed and analyzed by Western blotting using anti-p53 antibody. “p53-Ub” indicates the slower migrating species of the protein that are the ubiquitin-conjugated forms.

FIG. 5 shows that MEL23 decreases cell viability alone and in combination with DNA damaging agents. FIG. 5A shows that MEL23 specifically kills RKO cells and not RK0-E6 cells. RKO and RK0-E6 cells were plated in 384-well format and treated with a 10 μg/ml MEL23 for 48 hours. Alamar Blue was added for 16 hours and fluorescence was determined on a Victor3 Plate Reader. All values were normalized to no treatment. FIG. 5B shows that MEL23 synergizes with DNA-damaging agents. U2OS (p53 wild-type) and H1299 (p53 null) cells were plated in 384-well format and treated with the indicated compound for 48 hours. After the incubation period, Alamar Blue was added for 16 hours and fluorescence determined on a Victor3 Plate Reader. Percent cell death was determined by normalization to a no treatment control. Bliss Independence was determined by the formula C=A+B−A*B, where A is treatment with one compound alone and B is treatment with the second compound alone. Highlighted values in red have cell death greater than 50% in excess of Bliss Independence, values in orange have greater than or equal to 25%, and values in yellow have greater than or equal to 15%.

FIG. 6 shows the structure-activity relationship of MEL23 and MEL24. A series of MEL analogs were tested in the cell-based luminescence assay for activity. Cells were seeded in 384-well plates and treated with each MEL analog for 2 hours at the indicated concentrations. A luminescence buffer was added and the relative luminescence levels determined on a Victor3 Plate Reader. The fold induction of luminescence in the Mdm2-luciferase cell line at 15 μM is shown in the table. Each graph represents the median of three replicates normalized to a no treatment control.

FIGS. 7A and 7B show Mdm2 and p53 in vitro ubiquitination assays. Briefly, 50 ng of E1, 50 ng of Ubch5C (an E2 ligase), 1 μg of ubiquitin, 300 ng of Mdm2, and 50 ng of p53 were incubated for 1 hour at 37° C. in a ubiquitination buffer (50 mM TrisHCl pH 7.5, 5 mM MgCl₂, 2 mM NaF, 2 mM ATP, and 0.6 mM DTT). Samples were analyzed by Western blot and probed with either anti-Mdm2 or anti-p53 antibodies. PC indicates positive control and NC indicates negative control with one component of the reaction not included. Indicated samples were treated with 50 μg/ml of the indicated MEL compound.

FIG. 8 shows synergy data, including percent cell death with each treatment as described in FIG. 5B.

FIG. 9 shows a synthetic scheme for a radiolabeled MEL analog. This synthetic scheme can be generalized to generate other MEL23/24 analogs.

FIG. 10 shows that MEL23 increased the transcription of p53 target genes, Mdm2, p21, puma, and bax. RKO cells were treated with 5 μg/ml (14 μM) MEL23 for 24 or 48 hours. Quantitative real-time PCR was used to analyze the mRNA levels of the Mdm2, p21, puma, and bax transcripts. All values are normalized to no treatment, DMSO control. The figure is representative of three independent experiments and standard error bars represent intra-experimental error.

FIG. 11A shows that MEL23 inhibited Mdm2-MdmX hetero-complex E3 ligase activity in vitro. The bottom panel of each of 11A-C shows the results of ubiquitination assays in the presence of increasing concentrations of MEL compounds. These assays were performed with 100 ng of full-length Flag-Mdm2 or 200 ng of Flag-Mdm2/HA-MdmX complexes (100 ng of each protein as each are present in a 1:1 ratio, when purified), ³²P-labeled ubiquitin, UbCH5c, and E1. Following a 30-minute incubation at 37° C., reactions were terminated and resolved on a SDS-PAGE gel followed by autoradiography. PC indicates positive control, and NC indicates negative control with UbCH5C eliminated from the reaction. The gels were quantified by phosphoimaging using ImageQuant software (GE Healthcare Bio-Sciences Corp., Piscataway, N.J.). (Top Panel) FIG. 11B shows that MEL23 inhibited the Mdm2 RING-MdmX RING domain ligase activity in vitro. Reactions were performed as in FIG. 11A except 500 ng GST-Mdm2 (residues 400-491) and GST-MdmX (residues 400-490) were used for ubiquitin polymerization. FIG. 11C shows that MEL compounds do not affect Roc1/Cul1 E3 ligase activity in vitro. Reactions were performed as in FIG. 11A except with 3 μg of the Roc1/Cul1 ligase complex. Ubiquitination activity of the complex was tested in the presence of increasing concentrations of MEL23 and MEL24.

FIG. 12 shows that MEL23 alone decreased cell viability. FIG. 12A shows that MEL23 specifically decreased RKO cell viability. RKO and RKO-E6 cells were seeded in 384-well plates and treated with a dilution series of MEL23 for 48 hours as indicated. Alamar Blue was added for 16 hours and fluorescence determined on a Victor3 Plate Reader. The median percent inhibition of five replicates was determined and normalized to a no treatment control. One standard deviation is shown. FIG. 12B shows the percent survival of wild-type, p53^{-/-} and p53^{-/-}; mdm2^{-/-} mouse embryonic fibroblasts (MEFs). Cells were seeded in 384-well plates and treated with a dilution series of MEL23 for 48 hours at the indicated concentrations. Alamar Blue was added for 16 hours and fluorescence determined on a Victor3 Plate Reader. The median percent inhibition of five replicates was determined and normalized to a no treatment control. One standard deviation is shown.

FIG. 13 shows analysis of individual clones of Mdm2-luciferase cell lines. Three individual clones of each cell line, Mdm2-luciferase and Mdm2(C464A)-luciferase, were seeded in 384-well plates and treated with 5 μM MG132 for 2 hours. The Mdm2-luciferase clones are shown in black and the Mdm2(C464A)-luciferase clones are shown in gray. The graph is the median of 14 samples normalized to the no treatment of that clone. One standard deviation is shown. NT indicates a no treatment control.

FIG. 14 is a schematic for a representative synthesis of MEL24.

FIG. 15 shows a comparison of two independent syntheses of MEL24 on Mdm2 and p53 levels. U2OS cells (p53 wild-type) were treated with 5 μg/ml (15 μM) of MEL24 or the newly synthesized MEL24 from an independent source for 6 hours. Cells were lysed and analyzed by Western blotting using anti-Mdm2, anti-p53, and anti-eIF4E antibodies. NT indicates a no treatment control.

FIG. 16 shows the effects of MEL23, doxorubicin, and ALLN on p53 target genes. MEL23 increased the transcription of p53 target genes to intermediate levels compared to doxorubicin and ALLN. RKO cells were treated with 5 μg/ml (14 μM) MEL23 for 24 or 48 hours, 0.1 μg/ml (0.2 μM) doxorubicin for 48 hours, or 10 μM ALLN for 24 hours. Quantitative real-time PCR was used to analyze mRNA levels of the Mdm2, p21, puma, and bax transcripts. All values were normalized to no treatment (NT), DMSO control. The figure is representative of three independent experiments and standard error bars represent intra-experimental error.

FIGS. 17A-C show that MEL23 inhibits Mdm2, MdmX, and p53 ubiquitination in in vitro ubiquitination assays. Ubiquitin reactions contain 50 ng E1, 1 μg PK-ubiquitin, 50 ng His-UbCH5C, and 100 ng Flag-Mdm2. 50 ng His-p53 was used for in vitro p53 ubiquitination assay. 100 ng Flag-Mdm2 and 100 ng Ha-MdmX were used for ubiquitination reactions with the Mdm2-MdmX hetero-complex. After incubation for 30 minutes at 37° C., SDS sample buffer was added to the samples and they were boiled for 5 minutes. The products were resolved by SDS-PAGE and analyzed by Western blotting with anti-p53, anti-Mdm2 SMP14, 2A10, 3G5 mix, or anti-MdmX. Stars indicate locations where changes in the ubiquitination of the protein occur. The bottom panel of FIG. 17A shows the results of reactions as quantitated using an Odyssey imaging system. The graph represents the quantification of the Mdm2-ubiquitin species, normalized to the positive control. PC indicates positive control, and No E2 and No E3 are negative controls with the indicated protein eliminated from the reaction.

FIG. 18 shows that MEL23 increased Mdm2 in MEFs. MEFs were treated with 5 μg/ml (14 μM) of MEL23 for 6 hours. Cells were lysed and analyzed by Western blotting using anti-Mdm2 and anti-eIF4E antibodies. NT indicates a no treatment control.

FIG. 19 shows the percent survival of tumor-derived and non-transformed cell lines with MEL23 treatment. Cells were seeded in 384-well plates and treated with a dilution series of MEL23 for 48 hours at the indicated concentrations. Alamar Blue was added for 16 hours and fluorescence was determined on a Victor3 Plate Reader. The median percent growth inhibition of five replicates was determined and normalized to a no treatment control. One standard deviation is shown. Cell lines shown in blue, U2OS, RKO, and HCT116, are tumor-derived cell lines and cell lines shown in green, MCF10a and BJEH, are non-transformed cell lines. All cell lines are p53 wild-type.

FIG. 20 shows a representative synthetic scheme for compounds 12 and 19.

FIG. 21 shows a representative synthetic scheme for compound 14.

FIG. 22 shows a representative synthetic scheme for compounds 13, 15, and 16.

FIG. 23 shows a more detailed representative synthetic scheme for compound 19.

FIG. 24 shows an alternative synthetic scheme for compound 15.

FIG. 25 shows an alternative synthetic scheme for compound 14.

FIG. 26 shows an alternative synthetic scheme for compound 19.

FIG. 27 shows the structure and activity of MEL3 and MEL4.

FIG. 28 shows the structure and activity of MEL17.

FIG. 29 shows the effects of MEL23 on Mdm2 and p53 target genes. RKO cells were incubated in decreasing concentrations of MEL23, starting from 10 μg/ml (28 μM) for 6 hours.

FIG. 30 shows that MdmX is needed for efficient MEL24 activity. Bottom panel shows RKO cells, which, were transfected with siRNA against Mdm2 or luciferase as a control. Forty-eight hours after transfection, cells were treated with 10 μg/ml (30 μM) MEL24 or DMSO for 3 hours before harvesting. Lysates were separated by SDS-PAGE and probed with anti-MdmX, anti-p53 and anti-eIF4E antibody. The experiment was done in triplicates. Top panel shows fold change in p53 levels. Control luciferase-knockdown cells show a >6 fold increase in p53 levels MEL24 treatment, compared to <2 fold increase in MdmX knockdown cells. Error bars are SEM.

FIG. 31 shows that MEL compounds do not affect Roc1/Cul1 E3 ligase activity in vitro. Ubiquitination assays in the presence of increasing concentrations of MEL compounds were performed with 1.5 μg of the Roc1-Cul1 ligase complex, ³²P-labeled ubiquitin, UbCH5c, and E1. Following 30 min incubation at 37° C., reactions were terminated and resolved on a SDS-PAGE gel (FIG. 31A) followed by autoradiography and quantitation (FIG. 31B). PC indicated positive control, −E2 indicates no UbCH5C in the reaction and −E3 indicates Roc1/Cul1 eliminated from the reaction.

FIG. 32 shows that MEL23 increases the half-life of p53 and Mdm2. RKO cells were treated with 5 μg/ml (14 μM) for 4 hours, followed by 6, 4, 2, 1 hour or 30 minute treatment with 5 μg/ml (18 μM) cycloheximide. Cells were treated with MG132 (10 μM) for comparison. As shown in FIG. 32A, cell lysates were separated with SDS-PAGE and probed with anti-Mdm2, anti-p53 and anti-eIF4E. Protein levels were measured by densitometry using ImageJ and normalized to eIF4E. As shown in FIG. 32B, half life was calculated by plotting the remaining protein levels compared to cells not treated with cycloheximide.

FIG. 33 shows that MEL23 does not inhibit p53/Mdm2, p53/MdmX or Mdm2/MdmX complex formation. MCF7 cells were seeded in a 10-cm dish overnight and treated for 6 hours with 10 μg/ml (28 μM) MEL23, 5.3 μg/ml (10 μM) Nutlin-3a or DMSO. As shown in FIG. 33A, cell lysates were incubated with anti-p53 (pAB421) for 2 hours, followed by incubation with Protein-A agarose beads for an hour at 4° C. The experimental conditions of the results shown in FIG. 33B were as follows: FLAG-Mdm2 was transfected into cells and after 24 hours, treated with compounds, and cell lysates were incubated with anti-FLAG M2 antibody for 2 hours, followed by incubation with Protein-A/G sepharose beads for an hour at 4° C. Beads were washed and proteins eluted with 5×SDS and separated on SDS-PAGE.

FIG. 34 shows that MEL23 causes G2 cell cycle arrest. RKO, RKO E6, H1299 and MEF's were seeded and treated with designated concentration of MEL23. After two days incubation, cells were harvested and fixed in ethanol, followed by staining in a solution of 7.3 μM RNaseA and 98.7 μM propidium iodide (Sigma, St. Louis, Mo.). The samples were read on a FACS Calibur flow cytometer (BD Biosciences, San Jose, Calif.) and analyzed with CeliQuest software (BD Biosciences).

FIG. 35 shows a representative synthetic scheme for compound 20.

FIG. 36 shows a representative synthetic scheme for compound 21.

FIG. 37 shows a representative synthetic scheme for compound 22.

FIG. 38 shows a representative synthetic scheme for the 3S form of compound 23.

FIG. 39 shows a representative synthetic scheme for the 3R form of compound 23.

FIG. 40 shows a representative synthetic scheme for the 3S form of compound 24.

FIG. 41 shows a representative synthetic scheme for the 3R form of compound 24.

FIG. 42 shows a representative synthetic scheme for the 3S form of compound 25.

FIG. 43 shows a representative synthetic scheme for the 3R form of compound 25.

FIG. 44 shows a representative synthetic scheme for compound 26.

FIG. 45 shows a representative synthetic scheme for compound 27.

FIG. 46 shows a representative synthetic scheme for compound 28.

FIG. 47 shows a representative synthetic scheme for compound 29.

FIG. 48 shows a representative synthetic scheme for compound 30.

FIG. 49 shows a representative synthetic scheme for compound 31.

FIG. 50 shows an alternative synthetic scheme for compound 15.

FIG. 51 shows a representative synthetic scheme for compound 32.

FIG. 52 shows a representative synthetic scheme for compound 33.

FIG. 53 shows a representative synthetic scheme for compound 34.

FIG. 54 shows a representative synthetic scheme for compound 35.

FIG. 55 shows a representative synthetic scheme for compound 36.

### DETAILED DESCRIPTION OF THE INVENTION

One embodiment of the present invention is a compound according to formula (I):

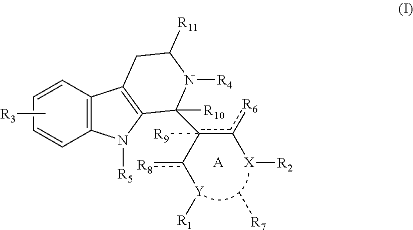

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₄alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
R₈ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, (2) R₄ is not methyl, and (3) R₃ is not methoxy;
   and with the third proviso that the compound is not:

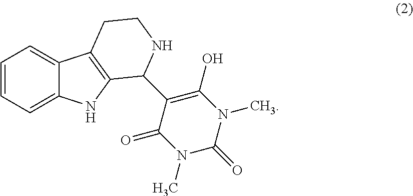

In one aspect of this embodiment, A is a six-membered ring. In another aspect of this embodiment, R₈, R₇, and R₈ are not all oxo. In a further aspect of this embodiment, R₁ and R₂ are independently selected from H, methyl, and phenyl. In yet another aspect of this embodiment, R₁ and R₂ are not both H. In an additional aspect of this embodiment, the compound is further not:

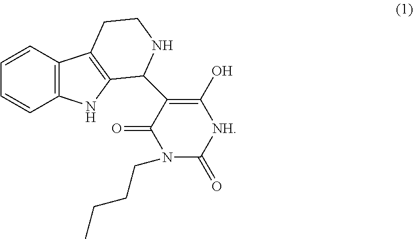

In another aspect of this embodiment, the compound has formula (II):

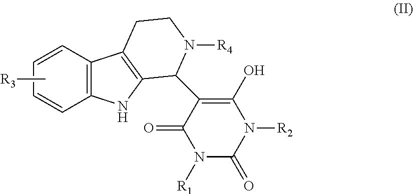

wherein:
R₁, R₂, R₃, and R₄ are independently selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

In a further aspect of this embodiment, the compound has formula (III):

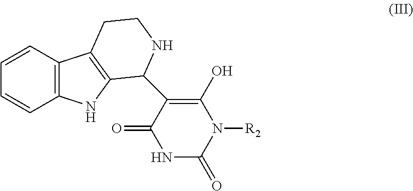

wherein
R₂ is selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carbon/late, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

In an additional aspect of this embodiment, the compound has formula (IV):

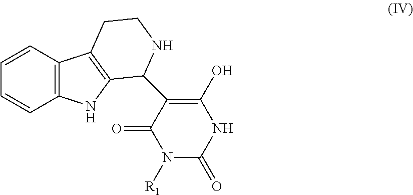

wherein
R₁ is selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

In yet another aspect of this embodiment, the compound has formula (V):

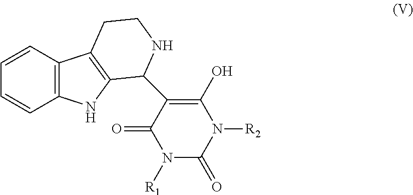

wherein
R₁ and R₂ are independently selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carbon/late, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

In another aspect of this embodiment, the compound has formula (VI):

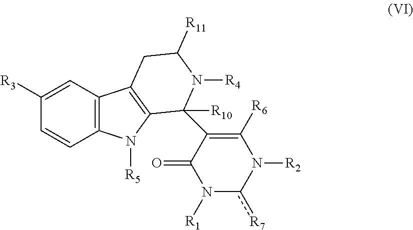

wherein:
R₁, R₂, R₃, R₄, R₅, R₆, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
a dashed line together with a solid line represent a single bond or a double bond;
wherein
if the bond linking a ring atom of A to R₇ is a double bond, R₇ is O; if the bond linking a ring atom of A to R₇ is a single bond, R₇ is selected from the group consisting of H, hydroxyl, C₁₋₈alkyl, C₁₋₄heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof.

In a further aspect of this embodiment, the compound is selected from the group consisting of:

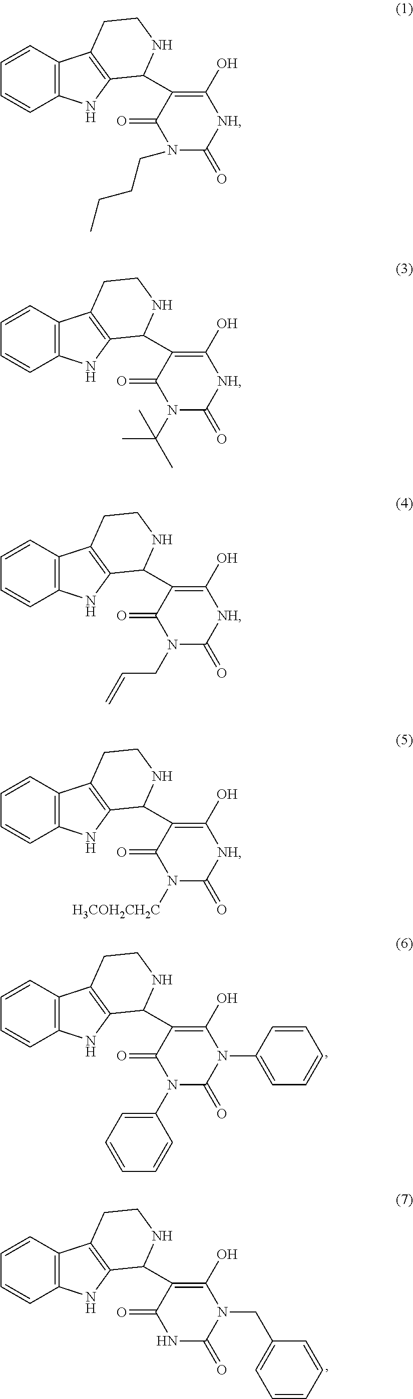

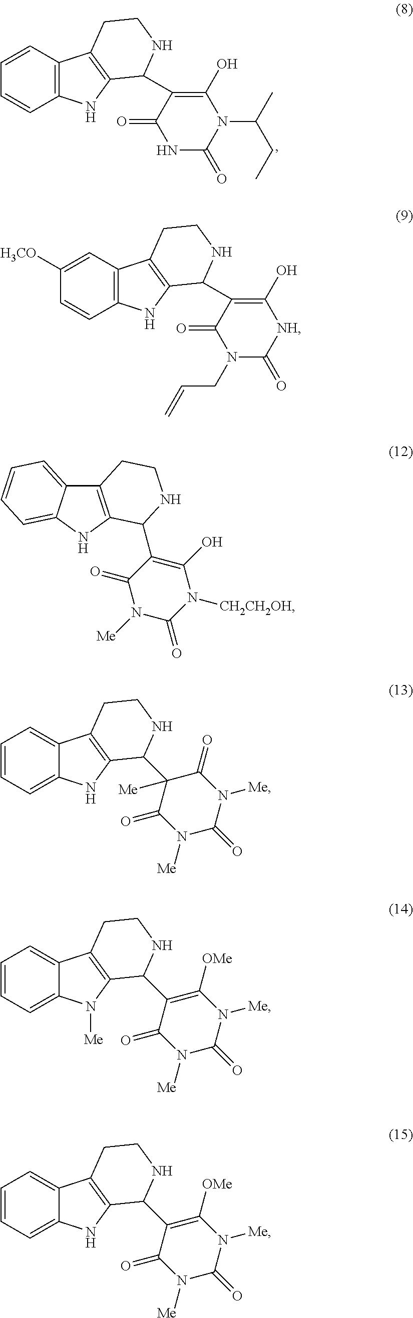

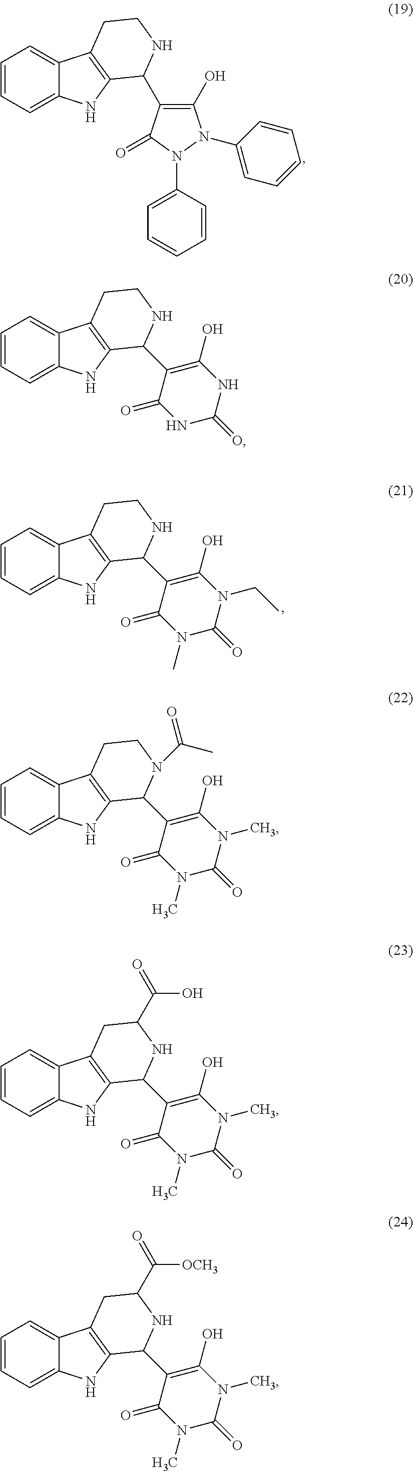

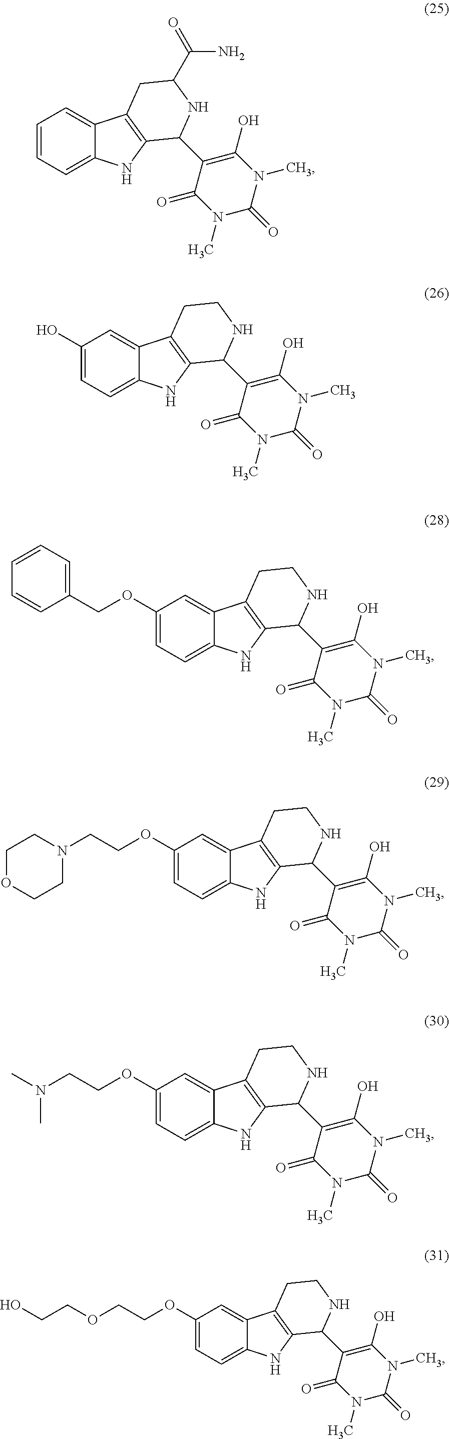

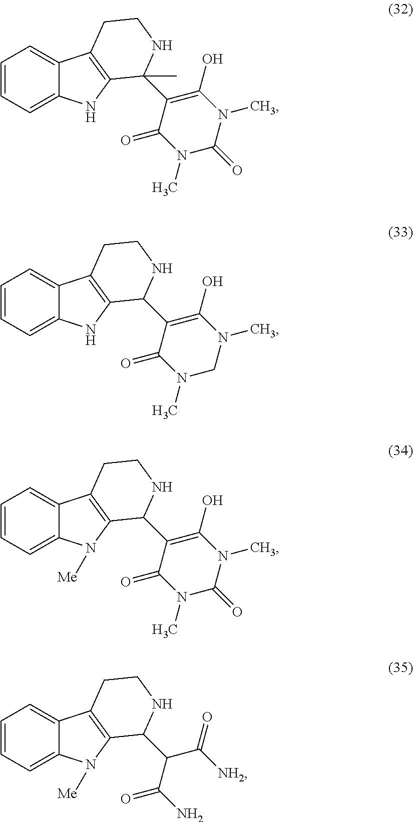

an enantiomer, optical isomer, diastereomer, N-oxide, crystalline form, hydrate, or pharmaceutically acceptable salt thereof.

Another embodiment of the present invention is a pharmaceutical composition. This pharmaceutical composition comprises a pharmaceutically acceptable carrier and a compound according to formula (I):

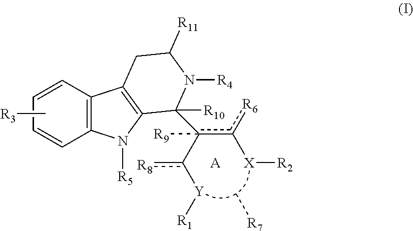

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, (2) R₄ is not methyl, and (3) R₃ is not methoxy;
   and with the third proviso that the compound is not:

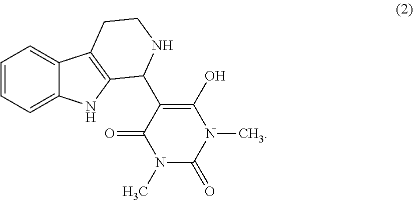

In one aspect of this embodiment, the compound is not:

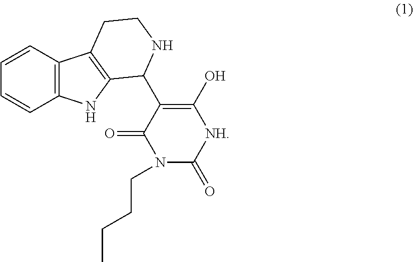

In another aspect of this embodiment, the pharmaceutical composition comprises a pharmaceutically acceptable carrier and a compound as disclosed above, which is selected from the group consisting of:

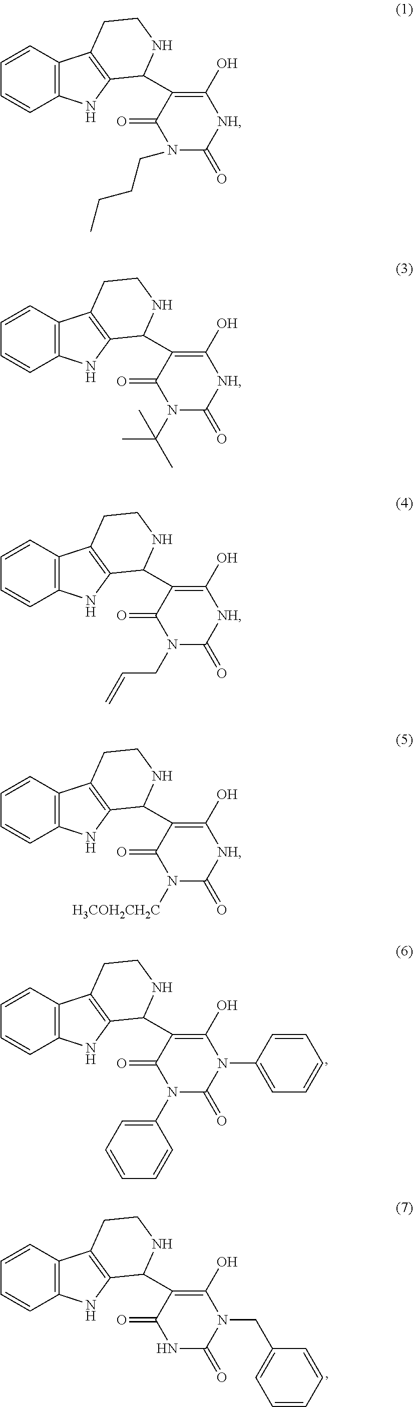

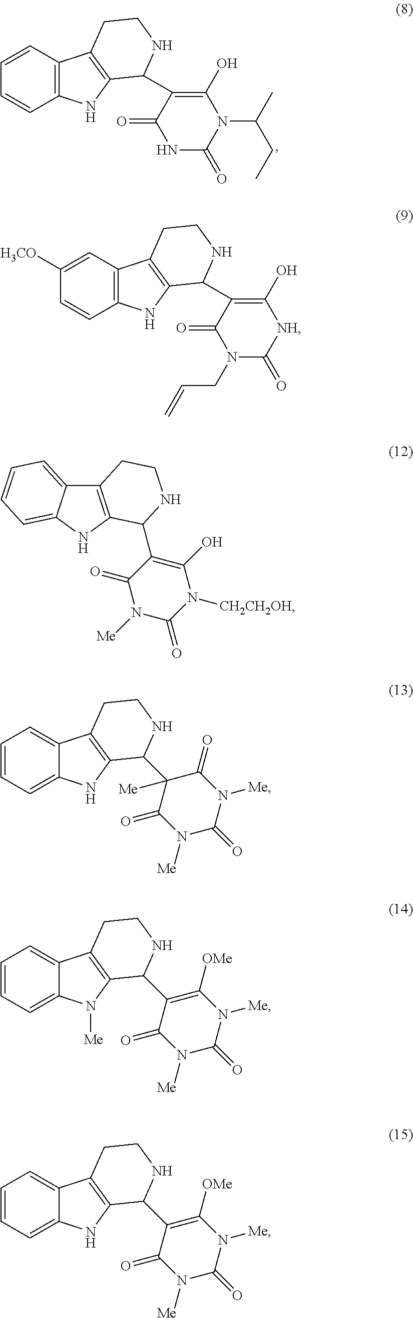

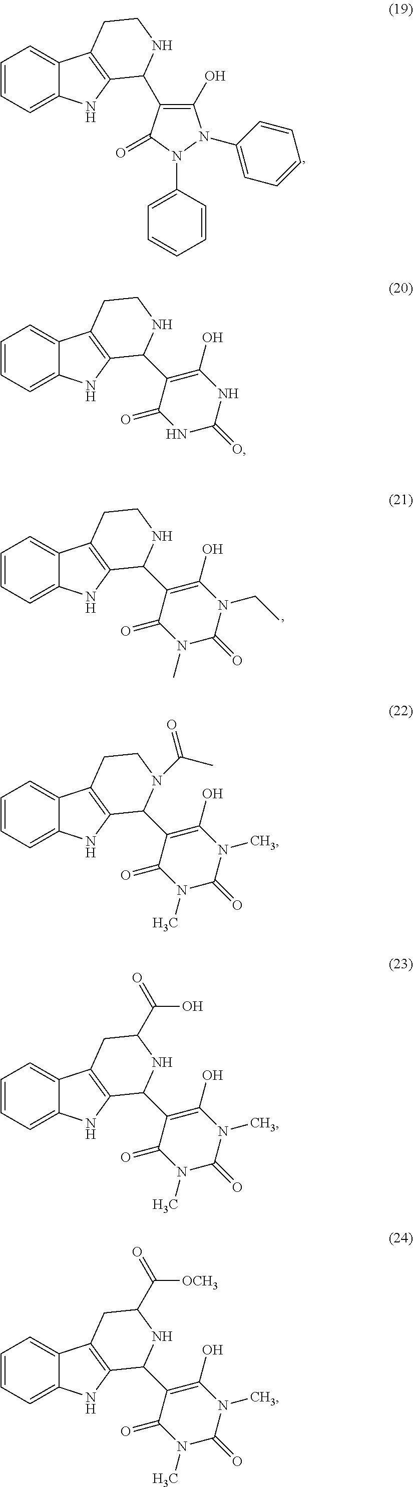

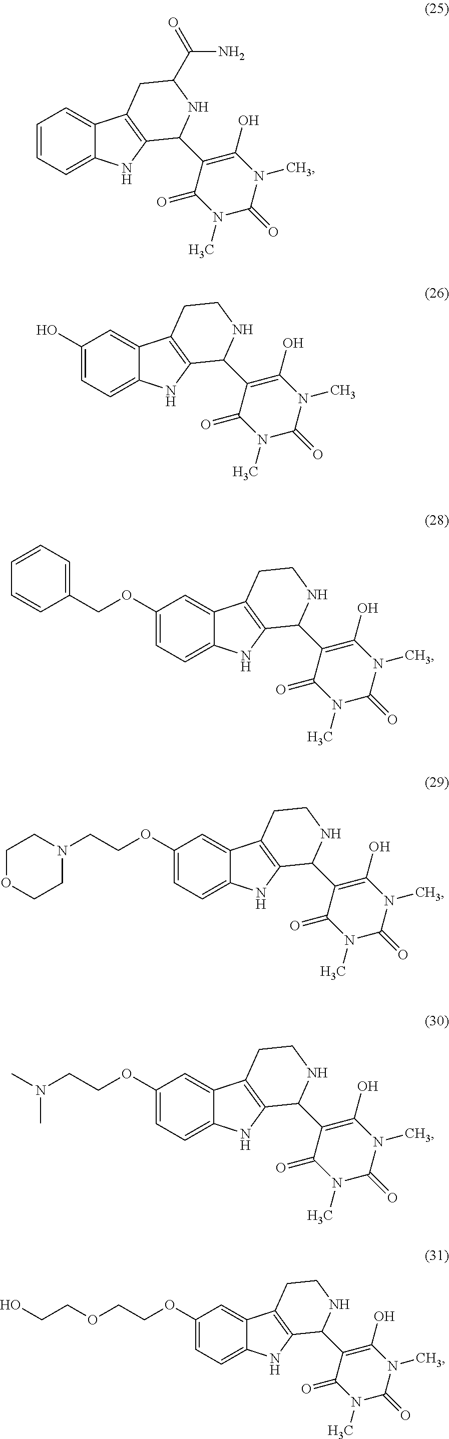

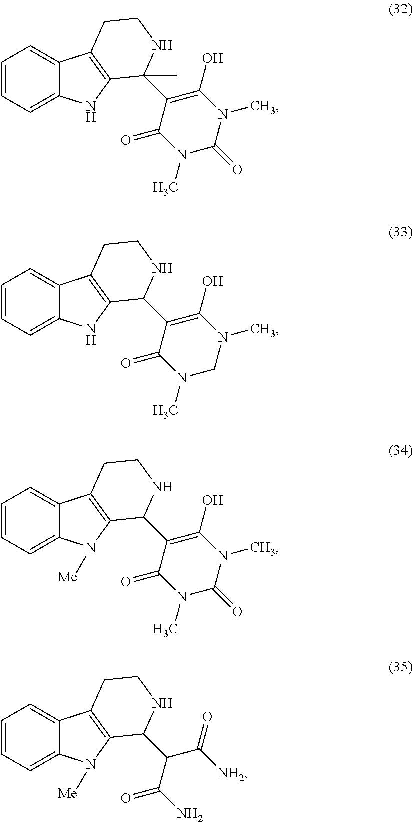

and an enantiomer, optical isomer, diastereomer, N-oxide, crystalline form, hydrate, or pharmaceutically acceptable salt thereof.

Another embodiment of the present invention is a pharmaceutical composition. This pharmaceutical composition comprises a compound according to the present invention, including, e.g., formulae (I)-(VI) compounds, a pharmaceutically acceptable carrier, and at least one DNA damaging agent. In the present invention, a “DNA damaging agent” is understood to mean any agent or treatment that induces DNA damage to a cell including UV irradiation, gamma irradiation, X-rays, alkylating agents, antibiotics that induce DNA damage by binding to DNA, inhibitors of topoisomerases, and any agent used in chemotherapy, which acts by causing DNA damage. Preferably, the DNA damaging agent is selected from the group consisting of actinomycin, amsacrine, anthracyclines, busulfan, cisplatin, cytoxan, epirubicin, hexamethylmelamineoxaliplatin, iphosphamide, mitoxantrone, taxotere, teniposide, triethylenethiophosphoramide, prednizone, dexamethasone, oxaliplatin, zoledronic acid, ibandronate, verapamil, podophyllotoxin, carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinbiastin, methotrexate, and combinations thereof.

A further embodiment of the present invention is a method for modulating murine double minute 2 protein (Mdm2) ligase activity. In the present invention, “modulating Mdm2 ligase activity” includes modulating the activity of Mdm2-MdmX hetero-complex E3 ligase. The modulation may occur by changing the expression level or degradation level of Mdm2 and/or the Mdm2-MdmX hetero-complex. Preferably, the modulation is inhibition of Mdm2-MdmX hereto-complex E3 ligase activity. In the present embodiment, the method comprises administering to a cell an amount of a compound according to formula (I):

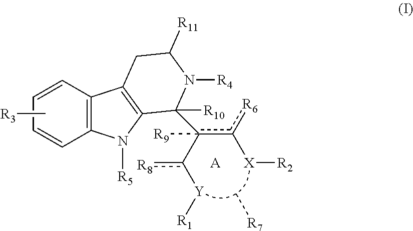

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁, are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, and (2) R₄ is not methyl; which amount is effective to modulate Mdm2 ligase activity. In this embodiment, preferably the Mdm2 ligase activity is Mdm2-MdmX hetero-complex E3 ligase activity.

In the present invention, an “effective” amount or “therapeutically effective” amount of a compound is an amount of such a compound that is sufficient to effect beneficial or desired results as described herein when administered to a cell, which may be a multicellular organism such as a mammal, preferably a human. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a compound according to the invention will be that amount of the compound, which is the lowest dose effective to produce the desired effect with no or minimal side effects.

A suitable, non-limiting example of a dosage of a compound according to the present invention is from about 1 ng/kg to about 1000 mg/kg, such as from about 1 mg/kg to about 100 mg/kg, including from about 5 mg/kg to about 50 mg/kg. Other representative dosages of a compound that modulates Mdm2 E3 activity, particularly Mdm2-MdmX hetero-complex E3 ligase activity, include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg. The effective dose of a compound may be administered as two, three, four, five, six or more sub-doses, administered separately at appropriate intervals throughout the day.

Another embodiment of the present invention is a method for treating or ameliorating the effects of a cancerous disease. This method comprises administering to a patient in need thereof an amount of a compound according to formula (I):

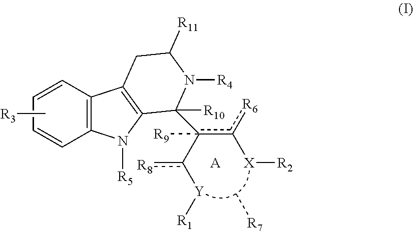

wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₇₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is single bond, R₈ is selected from the group consisting of H, hydroxyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
   or an enantiomer, optical isomer, diastereomer, N-oxide, crystalline form, hydrate, or pharmaceutically acceptable salt thereof,
   with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
   with the second proviso that (1) R₁ is not p-PhOCH₃, and (2) R₄ is not methyl;
   which amount is effective to treat or ameliorate the effects of the cancerous disease.

As used herein, a “cancerous disease” means a class of diseases in which a group of cells display uncontrolled growth (division beyond the normal limits). A cancerous disease may occur in any type of tissue and includes without limitation, colon cancer, breast cancer, bone cancer, colorectal cancer, lung cancer, pancreatic cancer, bladder cancer, skin cancer, liver cancer, lymphoma, and leukemia.

In one aspect of the methods according to the present invention, a DNA damaging agent as previously defined herein, including combinations thereof, is administered, e.g., separately or as a part of a pharmaceutical composition that includes a compound of the invention, including compounds of formula (I)-(VI).

In an additional aspect of the methods according to the present invention, the compound is selected from the group consisting of:

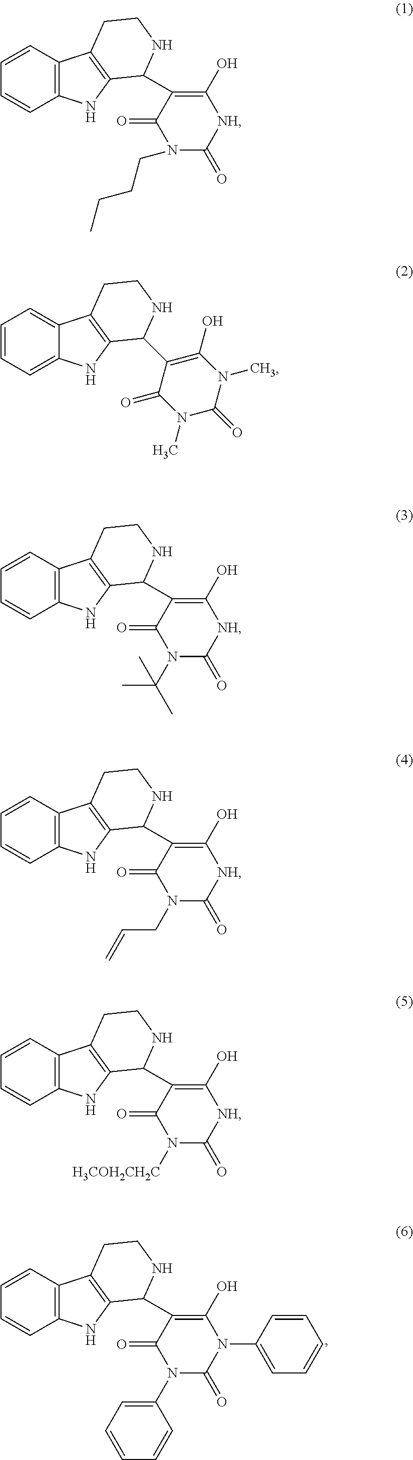

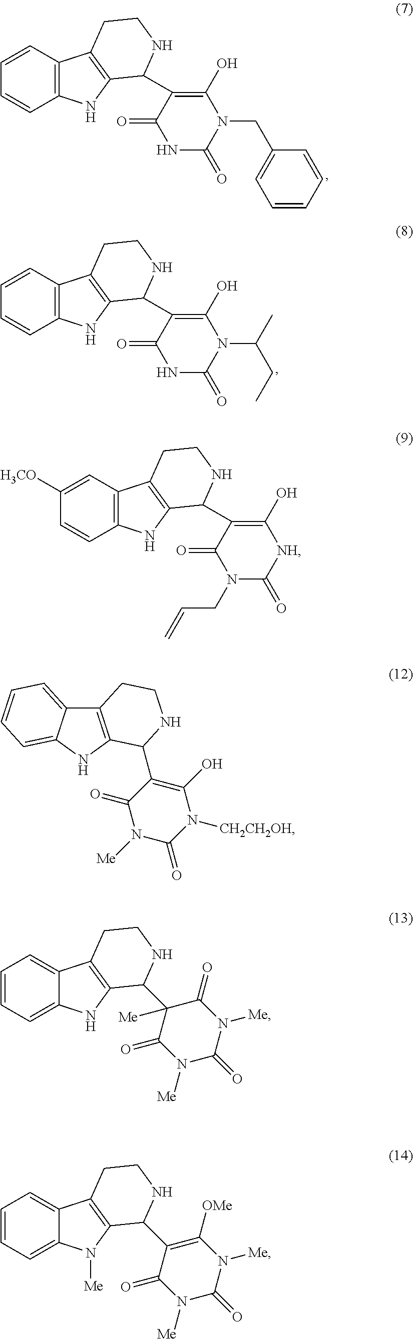

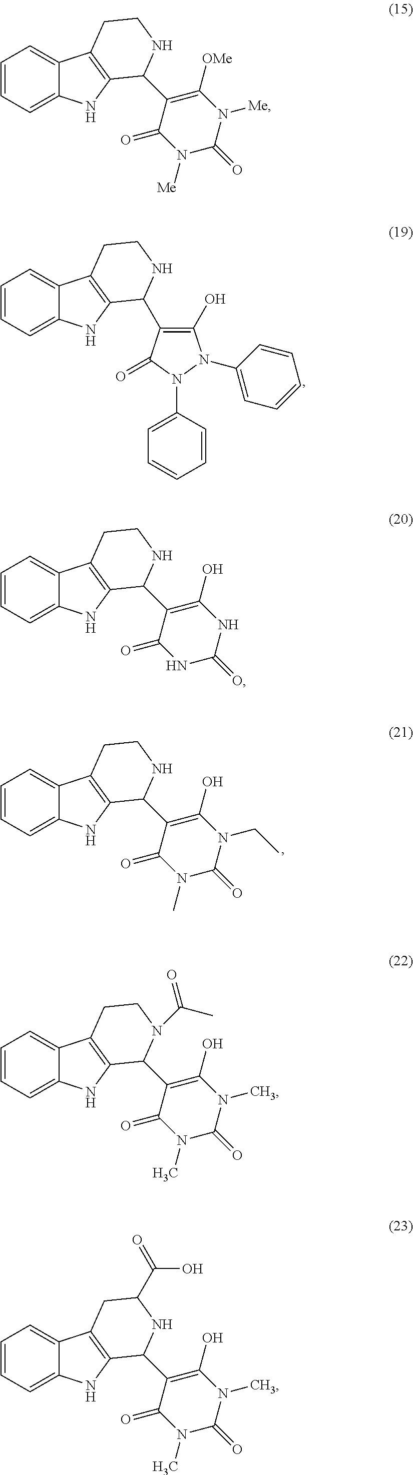

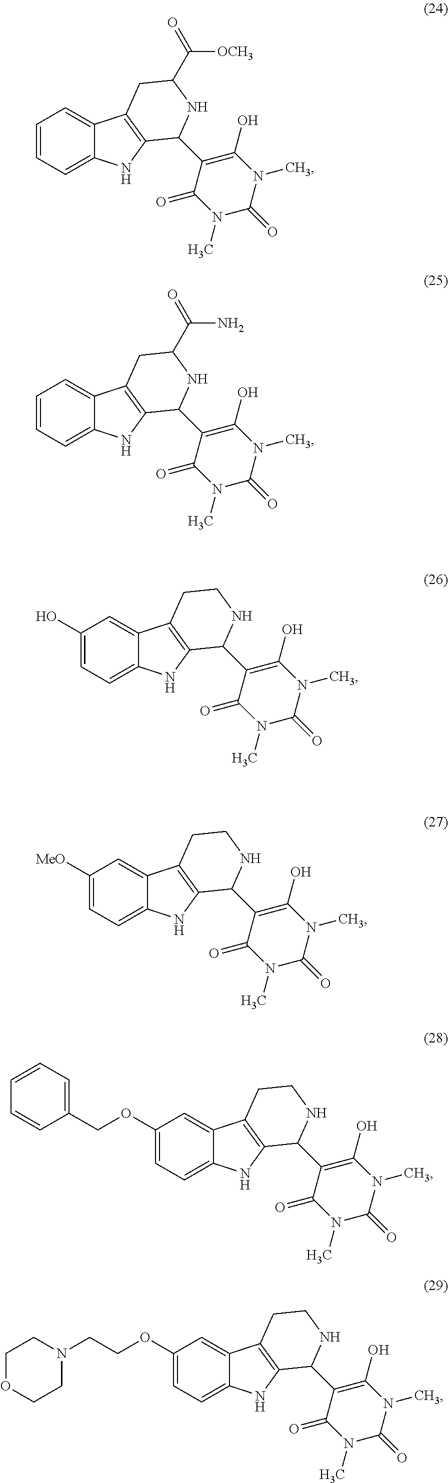

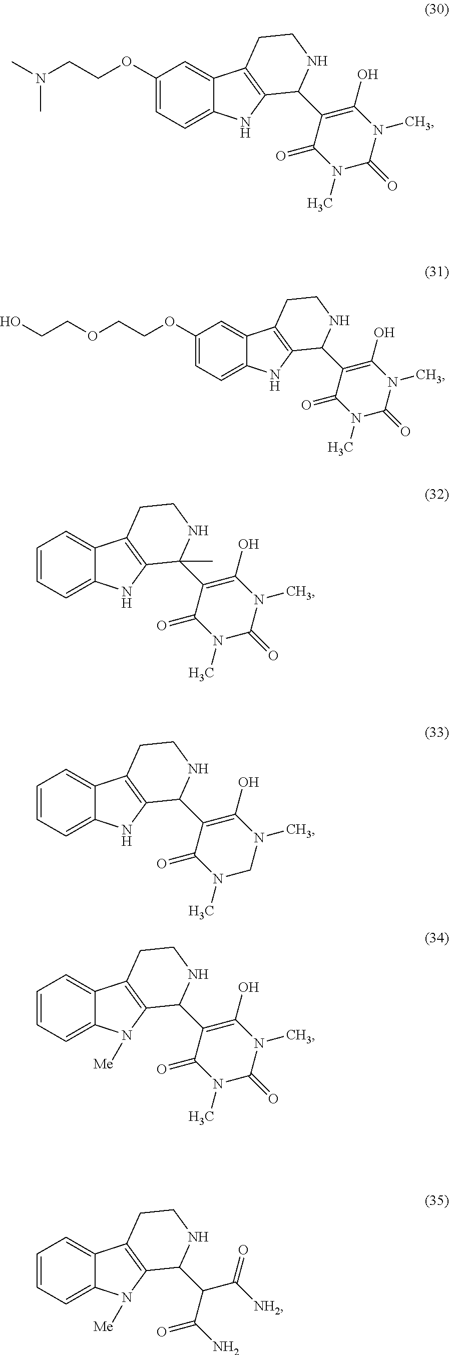

and an enantiomer, optical isomer, diastereomer, N-oxide, crystalline form, hydrate, or pharmaceutically acceptable salt thereof.

In yet another aspect of the methods according to the present invention, the compound is further not:

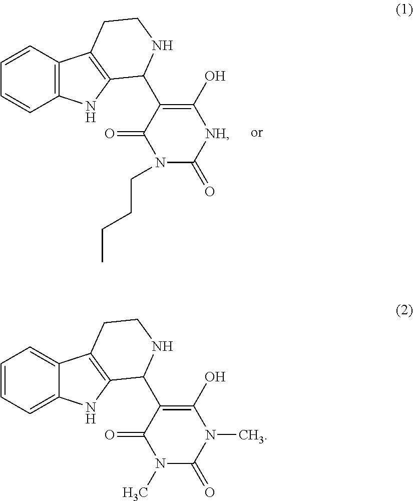

In a further aspect of the methods according to the present invention, wherein the compound is further not:

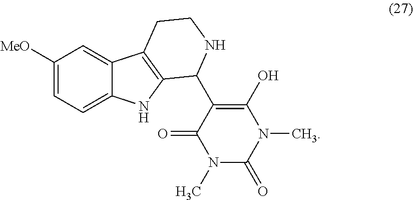

In an additional aspect of the methods according to the present invention, A of the compound having formula (I) is a six-membered ring. In a further aspect of methods according to the present invention, R₆, R₇, and R₈ of the compound having formula (I) are not all oxo. In another aspect of the methods according to the present invention, R₁ and R₂ of the compound having formula (I) are independently selected from H, methyl, and phenyl. In yet another aspect of the methods according to the present invention, R₁ and R₂ of the compound having formula (I) are not both H. In an additional aspect of the methods according to the present invention, R₃ of the compound having formula (I) is not methoxy.

Another embodiment of the present invention is a compound having the structure of

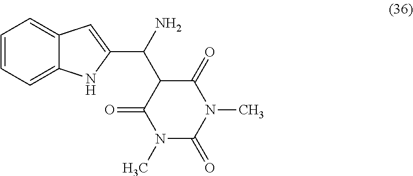

or an enantiomer, optical isomer, diastereomer, N-oxide, crystalline form, hydrate, or pharmaceutically acceptable salt thereof.

An additional embodiment of the present invention is a pharmaceutical composition comprising the compound 36 disclosed above.

A further embodiment of the present invention is a method for modulating murine double minute 2 protein (Mdm2) ligase activity. This method comprises administering to a cell an amount of a compound 36 disclosed above, which amount is effective to modulate Mdm2 ligase activity.

Yet another embodiment of the present invention is a method for treating or ameliorating the effects of a cancerous disease. This method comprises administering to a patient in need thereof an amount of a compound 36 disclosed above, which amount is effective to treat or ameliorate the effects of the cancerous disease.

The pharmaceutical compositions of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a pharmaceutical composition of the present invention may be administered in conjunction with other treatments. A pharmaceutical composition of the present invention may be encapsulated or otherwise protected against gastric or other secretions, if desired.

The pharmaceutically acceptable compositions of the invention comprise one or more active ingredients in admixture with one or more pharmaceutically-acceptable carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention may, optionally, contain additional ingredients and/or materials commonly used in such pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically-acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable carrier. The ointments, pastes, creams and gels may contain excipients. Powders and sprays may contain excipients and propellants.

Pharmaceutical compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

In the foregoing embodiments, the following definitions apply.

As used herein, the term “acyl” has its art-recognized meaning and refers to a group represented by the general formula hydrocarbylC(O)—, preferably alkylC(O)—.

As used herein, the term “acylamino” has its art-recognized meaning and refers to an amino group substituted with an acyl group and may be represented, for example, by the formula hydrocarbylC(O)NH—.

As used herein, the term “alditol” means any of a class of acyclic alcohols containing multiple hydroxyl groups, which are derived from a monosaccharide containing a terminal carbonyl group and having a chemical formula of the form Cₙ(H₂O)ₙ by reduction of the carbonyl functional group. Alditol includes, for example, sorbitol.

The term “alkoxy” refers to an alkyl group, preferably a lower alkyl group, having an oxygen attached thereto. Representative alkoxy groups include methoxy, ethoxy, propoxy, isopropoxy, tert-butoxy and the like. Other alkoxy groups within the scope of the present invention include, for example, the following:

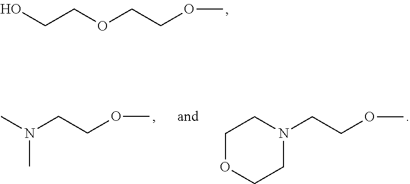

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 8 or fewer carbon atoms in its backbone (e.g., C₁-C₈ for straight chains, C₃-C₈ for branched chains). Likewise, preferred cycloalkyls have from 3-8 carbon atoms in their ring structure, including 5, 6 or 7 carbons in the ring structure.

Moreover, unless otherwise indicated, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, alkyl, alkenyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group. The term “alkylaryl”, as used herein, refers to an aryl group substituted with an alkyl group. The term “alkylheteroaryl”, as used herein, refers to a heteroaryl group substituted with an alkyl group.

The term “alkylheterocycle”, as used herein, refers to an heterocycle group substituted with an alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “acetamino”, as used herein, refers to the group CH₃CONH—.

The term “amide”, as used herein, refers to a group

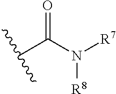

wherein R⁷ and R⁸ each independently represent a hydrogen or hydrocarbyl group, or R⁷ and R⁸ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

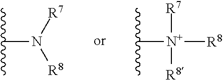

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure.

The term “amino acid,” as used herein, refers a functional group containing both amine and carboxyl groups.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

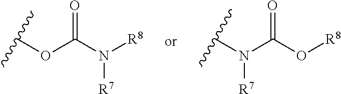

wherein R⁷ and R⁸ independently represent hydrogen or a hydrocarbyl group.

The terms “carbocycle”, “carbocyclyl”, and “carbocyclic”, as used herein, refer to a non-aromatic saturated or unsaturated ring in which each atom of the ring is carbon. Preferably a carbocycle ring contains from 3 to 8 atoms, including 5 to 7 atoms, such as for example, 6 atoms.

The term “carbohydrate”, as used herein, is a functional group that includes an aldehyde or ketone group with many hydroxyl groups added to the carbon backbone, usually one on each carbon atom that is not part of the aldehyde or ketone functional group. Carbohydrate include monosaccharides such as glucose, galactose, and fructose. Carbohydrate also include oligosaccharides made of two or more monosaccharides, but preferably two monosaccharides.

The terms “carboxy” and “carboxyl”, as used herein, refer to a group represented by the formula —CO₂H.

The term “carboxylate” refers to the conjugate base of a carboxyl group, represented by the formula —COO⁻.

The term “ester”, as used herein, refers to a group —C(O)OR⁷ wherein R⁷ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heteroalkyl” means an alkyl in which at least one carbon of a hydrocarbon backbone is substituted with a heteroatom. Heteroalkyls include alkoxyalkyls, such as C₁₋₈ alkoxyalkyl.

The term “heteroaromatic” means at least one carbon atoms in the aromatic group is substituted with a heteroatom.

The terms “heterocyclyl”, “heterocycle”, “heterocyclic”, and the like refer to substituted or unsubstituted non-aromatic ring structures, preferably 3- to 8-membered rings, whose ring structures include at least one heteroatom, preferably one to four heteroatoms, more preferably one or two heteroatoms. The terms “heterocyclyl,” “heterocyclic,” and the like also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “hydroxyl,” as used herein, refers to the group —OH.

The term “lower” when used in conjunction with a chemical moiety, such as, acyl, alkyl, alkenyl, alkynyl, or alkoxy is meant to include groups where there are ten or fewer non-hydrogen atoms in the substituent, preferably eight or fewer, such as for example, from about 2 to 8 carbon atoms, including less than 6 carbon atoms. A “lower alkyl”, for example, refers to an alkyl group that contains ten or fewer carbon atoms, preferably eight or fewer. In certain embodiments, acyl, alkyl, alkenyl, alkynyl, or alkoxy substituents defined herein are respectively lower acyl, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 3 to 8, such as for example, 5 to 7.

The term “oxo” refers to the group ═O.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As used herein, the term “substituent,” means H, cyano, oxo, nitro, acyl, acylamino, halogen, hydroxy, amino acid, amine, amide, carbamate, ester, ether, carboxylic acid, thio, thioalkyl, thioester, thioether, C₁₋₈ alkyl, C₁₋₈alkoxy, C₁₋₈alkenyl, C₁₋₈aralkyl, 3- to 8-membered carbocyclic, 3- to 8-membered heterocyclic, 3- to 8-membered aryl, or 3- to 8-membered heteroaryl, sulfate, sulfonamide, sulfoxide, sulfonate, sulfone, alkylsulfonyl, and arylsulfonyl.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

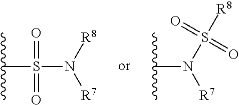

wherein R⁷ and R⁸ independently represents hydrogen or hydrocarbyl.

The term “sulfoxide” is art-recognized and refers to the group —S(O)—R⁷, wherein R⁷ represents a hydrocarbyl.

The term “sulfonate” is art-recognized and refers to the group SO₃H, or a pharmaceutically acceptable salt thereof.

The term “sulfone” is art-recognized and refers to the group —S(O)₂—R⁷, wherein R⁷ represents a hydrocarbyl.

The term “thio”, as used herein, refers to the —SH group.

The term “thioalkyl”, as used herein, refers to an alkyl group substituted with a thiol group.

The term “thioester”, as used herein, refers to a group —C(O)SR⁷ or —SC(O)R⁷ wherein R⁷ represents a hydrocarbyl.

The term “thioether”, as used herein, is equivalent to an ether, wherein the oxygen is replaced with a sulfur.

The term “urea” is art-recognized and may be represented by the general formula

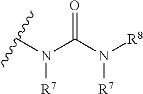

wherein R⁷ and R⁸ independently represent hydrogen or a hydrocarbyl.

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers, optical isomers, and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that compounds of the present invention having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., “Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

In the present invention, free bases of formulae (I)-(VI), their stereoisomers may be converted to the corresponding pharmaceutically acceptable salts under standard conditions well known in the art. For example, a free base of formula (I) is dissolved in a suitable organic solvent, such as methanol, treated with one equivalent of maleic or oxalic acid for example, one or two equivalents of hydrochloric acid or methanesulphonic acid for example, and then concentrated under vacuum to provide the corresponding pharmaceutically acceptable salt. The residue can then be purified by recrystallization from a suitable organic solvent or organic solvent mixture, such as methanol/diethyl ether.

The N-oxides of compounds of formulae (I)-(VI), may be synthesized by simple oxidation procedures well known to those skilled in the art. For example, the oxidation procedure described by P. Brougham et al. (Synthesis, 1015 1017, 1987), allows the two nitrogen of a piperazine ring to be differentiated, enabling both the N-oxides and N,N′-dioxide to be obtained.

The following examples are provided to further illustrate the methods of the present invention. These examples are illustrative only and are not intended to limit the scope of the invention in any way.

### EXAMPLES

### Example 1

### Materials and Methods

### Reagents

MG132 (Sigma, St. Louis, Mo., Cat. No. C2211), ALLN (Calbiochem, San Diego, Calif., Cat. No. 208719), cycloheximide (Sigma, Cat. No. C7698), doxorubicin hydrochloride (Sigma, Cat. No. D1515), etoposide (Sigma, Cat. No. E1383), camptothecin (Sigma, Cat. No. C9911), and Nutlin-3 (Sigma, Cat. No. N6287) were used for cell treatments at the indicated concentrations.

Antibodies to p53 (Calbiochem, Cat. No. OP43), Mdm2 (Calbiochem, Cat. No. OP115), phospho-p53 ser15 (Cell Signaling Technology, Danvers, Mass., Cat. No. 9284s), MdmX (Bethyl Laboratories, Montgomery, Tex., Cat. No. A300-287A), eIF4E (BD Biosciences, San Jose, Calif., Cat. No. 610270), and actin (Santa Cruz Biotech, Santa Cruz, Calif., Cat. No. sc-1616-R) were used. Anti-Mdm2 SMP14 (Millipore, Billerica, Mass.), 2A10 (Calbiochem), and 3G5 (Calbiochem) mix, as supernatants from hybridoma cultures, were used for Mdm2 ubiquitination assays.

### Cell Lines and Transfections

293T (human embryonic kidney), H1299 (non-small cell lung carcinoma), MCF7 (mammary gland adenocarcinoma), wild-type MEFs, p53^{-/-} MEFs, and p53^{-/-} mdm2^{-/-} MEFs were maintained in Dulbecco's modified Eagle's medium (DMEM) (Fisher Scientific, Pittsburgh, Pa., Cat. No. MT-15-018-CM) with 10% fetal bovine serum (FB) and 100 μg/ml penicillin/streptomycin (P/S). Mdm2-luciferase and Mdm2(C464A)-luciferase stable cell lines (in 293T cells) were additionally treated with 300 μg/ml of zeocin (Invitrogen, Carlsbad, Calif., Cat. No. R250-05). Wild-type MEFs were a kind gift from Craig B. Thompson at the University of Pennsylvania. P53^{-/-} MEFs, and p53^{-/-}; mdm2^{-/-} MEFS were kind a gift from Guillermina Lozano at the University of Texas. RKO (colon carcinoma) and RKO E6 (colon carcinoma) cells were maintained in Minimum Essential Medium (MEM) Eagle (Sigma, Cat. No. M5650) with 10% FB and 100 μg/ml P/S. U2OS (osteosarcoma) and HCT116 (colorectal carcinoma) were maintained in McCoy's 5a Medium (Invitrogen, Cat. No. 16600-108) with 10% FB and 100 μg/ml P/S. All cells were grown at 37° C. in 5% CO₂.

Cells were transfected using FuGENE 6 (Roche, Nutley, N.J., Cat. No. 814-442-001) in accordance with the manufacturer's protocol.

To knockdown MdmX expression, cells were transfected with 20 nM control siRNA (AACTTACGCTGAGTACTTCGA) (SEQ ID NO: 19) or MdmX siRNA (AGAGATTCAGCTGGTTATTAA) (SEQ ID NO: 20) using DharmaFECT1 (Dharmacon, add catalog number) according to manufacturer's instructions. After 48 hours of knockdown, cells were treated with compounds.

### Plasmids

Mdm2-luciferase and Mdm2(C464A)-luciferase plasmids were cloned into the pcDNA3.1 vector (Invitrogen, Cat. No. P/N 35-0574) using the Nhe 1 and Xho 1 restriction sites. Luciferase is fused to the N-terminus of Mdm2 with a linker (Gly Ser Gly Gly Gly Ser Gly Gly Gly Gly Ser Gly Gly Gly Gly Ser Gly Gly Thr Gly Ser, SEQ ID NO:1) inserted between the Mdm2 and the luciferase DNA sequence. This was confirmed by sequencing. Primer sequences for cloning are set forth below.

Flag-Mdm2 in pcDNA3 (34), HA-Ubiquitin in pcDNA3 (34), and p53 in pcDNA3 (59) have been previously described. PK-ubiquitin (35) and His-UbCH5c (35) in pET-15a, His-p53 in pRSETB (60), GST-Mdm2 (400-491) (35), and GST-MdmX (410-491) (35) in pGGEX-4T1 have also been previously described. For constructing HA-MdmX baculovirus, the PCR reaction was performed to insert an HA tag upstream of MdmX. The primers used for the PCR reaction are listed as SEQ ID NOs: 17-18. The PCR product was purified and digested with Rsrll and KpnI and cloned into the pFastBac HTa plasmid (Invitrogen). Flag-Mdm2 baculovirus was a kind gift from Dr. S. Grossman at the University of Massachusetts Medical School. Both Flag-Mdm2 and HA-MdmX baculoviruses were prepared according to the Bac-to-Bac baculovirus expression system (Invitrogen).

### Compound Libraries

51,356 compounds were screened. These compound included synthetic compounds and natural products from InterBioScreen Ltd. (Chemogolovka, Russia), Timtec LLC (Newark, Del.), Chembridge Corp. (San Diego, Calif.), MicroSource Discovery Systems Inc. (Gaylordsville, Conn.), and Life Chemicals Inc. (Burlington, Canada). These compounds were stored in dimethylsulfoxide (DMSO) in 384-well plates at 4 mg/ml. MEL23 and MEL24 were identified from InterBioScreen and the analogs were also obtained from InterBioScreen.

An additional 218,724 compounds were screened through the National Institutes of Health (NIH) Molecular Libraries Screening Centers Network (MLSCN) through an RO3 grant.

### Screening

The Mdm2-luciferase cell line was seeded at 7,500 cells per well, in 384-well white plates (PerkinElmer, Waltham, Mass., Cat. No. 6007688), in 27 μl of media (DMEM with FBS and P/S). Assay plates with cells were incubated at 37° C. overnight to allow cells to adhere. After 24 hours, 2 μl from the ‘mother’ plate (4 mg/ml) was transferred to a ‘daughter plate’ containing 148 μl media in order to dilute the compound 1:75. Next, 3 μl from each well of the ‘daughter plates’ was added to triplicate assay plates for a final concentration of 5.33 μg/ml. Assay plates were incubated at 37° C. for 2 hours. Then the cells were lysed with the addition of 30 μl of luminescence buffer (PerkinElmer, Cat. No. 6016989), and the plates were incubated at room temperature for 30 minutes before being analyzed on a Victor3 Plate Reader (PerkinElmer) for luminescence. Compounds that caused an increase in luminescence greater than 30% were re-ordered for further analysis. All repurchased compounds were tested in a 2-fold dilution series via the same protocol as the primary screening. All transfers were conducted using a BioMek (Beckman Coulter, Fullerton, Calif.). The final optimized screen has a z′ value of 0.63 (65). This was determined by comparing the negative control, no treatment, to the positive control, 10 μM MG132.

### Screening (MLSCN Library)

Detailed description may be found in Pubchem (BioAssay AID 1230, AID 1442, AID 1444, and AID 1394, which are incorporated in full herein) and are summarized below. The final optimized screen has a z′ value of 0.49.

For the screens, DMEM (Cat. No. 11995-081), Fetal Bovine serum (Cat. No. 26140-079), Streptomycin (Cat. No. 15140-155), Trypsin-EDTA (Cat. No. 25200-106) and Zeocin (Cat. No. R250-01) were all purchased from Invitrogen. SteadyLite HTS gene assay reporter system (Cat. No. 6016989) was purchased from Perkin Elmer. The Luminescence assay was carried out in 1536-well white plates from Corning (Cat. No. 3727). Breathe-easy membranes (Cat. No. Z380059) were from Sigma.

### AID 1230

HTS was performed using 218,724 compounds of the MLSCN library individually plated into 10 μl 1536 compound plates at a concentration of 2.5 mM each, which were diluted 500-fold into 3 μl 1536 well assay plates (final concentration of compound in assay plate: 5 μM).

Cells were plated and allowed to settle at 37° C. for 4 hours. The cells were then incubated with 5 μM compound (final DMSO concentration: 0.2%) at 37° C. for 2 hours. SteadyLite luciferase reagent was added, plates incubated for 15 minutes and then read on Envisions reader (Perkin Elmer).

1536 well plates were filled with 3 μl of cells in DMEM (2000 cells per well) using Aquamax DW4 (Molecular Devices, Sunnyvale, Calif.). 3 μl DMEM were added to columns 1, 2, 45, and 46 using Aquamax DW4. Plates were sealed with Breathe-easy membranes and incubated at 37° C. for 4 hours. 6 nL of compound (0.25 mM in DMSO) were added using Evolution™ 1536 pintool (Perkin Elmer). Plates were sealed with Breathe-easy membranes again and incubated at 37° C. for 2 hours. Then, 3 μl SteadyLite HTS reagent were added, and the mixture was incubated at room temperature for 15 minutes. Luminescence was read on Envision reader.

The data was analyzed in IDBS ActivityBase (ID Business Solutions Ltd., Guildford, Surrey, United Kingdom). Each HTS plate had a single test compound (5 μM in 0.2% DMSO) in columns 5-44, controls (cells, no compound) in columns 3, 4, 47, and 48, and blanks (DMEM) in columns 1, 2, 45, and 46. Percent enhancement of signal was calculated for each compound from the signal in luminescence units (FU) and the mean of the plate controls and the mean of the plate blanks using the following equation:

% enhancement=100*(((signal−blank mean)−(control mean−blank mean))/(control mean−blank mean)))

Activity scores were calculated as follows:
For positive percent enhancement, score=0.4×Percent inhibition

For negative percent enhancement, score=0

Activity outcome is reported as follows:
(1) Percent enhancement>=30=active
(2) Percent enhancement<30=inactive

A hit cut-off of 30% enhancement was selected. Based on this cutoff, a hit rate of 0.078% was observed.

### AID 1394

Cells were plated and allowed to settle at 37° C. overnight. The cells were then incubated with different concentrations of compound (final DMSO concentration was 0.25%) at 37° C. for 2 hours. SteadyLite luciferase reagent was added, plates incubated for 30 minutes and then read on Envision® reader.

384 well plates were filled with 24 μl of cells in DMEM (7500 cells per well) using Wellmate (Chardon, Ohio) (all columns except 1 and 23). 24 μl DMEM were added to columns 1 and 23 using Wellmate. Then, plates were sealed with Breathe-easy membranes and incubated at 37° C. overnight. 1 μl of compound was diluted from dose-response plate with 79 μl DMEM to get a diluted dose-response plate (top concentration 125 μM). Then, 6 μl of diluted compound were added to the overnight-grown cells, sealed with Breathe-easy membrane and incubated at 37° C. for 2 hours. Afterwards, 30 μl SteadyLite HTS reagent was added, and the mixture was incubated at room temperature for 30 minutes. Finally, luminescence was read on Envision® reader.

The data was analyzed in IDBS ActivityBase. Each dose-response plate contained compounds in columns 3-22, controls (cells, no compound) in columns 2 and 24, and blanks (no cells) in columns 1 and 23. Each column 3-22 contained 16 two-fold dilutions of a single compound, ranging in concentration from 25 μM to 0.75 nM. Percent enhancement of signal was calculated for each well from the signal in luminescence units (LU) and the mean of the plate controls and the mean of the plate blanks using the following equation:

% enhancement=100*(((signal−blank mean)−(control mean−blank mean))/(control mean−blank mean)))

Dose response curves of percent enhancement were fit using the XLfit equation 205 (four parameter logistic model) (ID Business Solutions Ltd.).

The activity score reported here is based on follow-up IC₅₀ testing on compounds that showed >30% inhibition in the primary HIS. IC₅₀ scores were calculated based on the maximum percent enhancement and the EC₅₀ value as follows:
(1) For EC₅₀>0, Score=maximum percent enhancement/2
(2) For EC₅₀=0 (bad curve fit), Score=0

Compounds that gave percent inhibition >30% in the primary HTS were judged to be hits and these compounds were selected for follow-up dose-response testing. Maximum percent enhancement shown by each compound is reported and is used to determine potency of the compound along with the EC₅₀ value.

Activity outcome is reported as follows:

(1) EC₅₀>0 and maximum percent enhancement>35=Active

(2) EC₅₀>0 and maximum percent enhancement<35=Inactive

Hence, active compounds have a score >18, and inactive compounds have a score <18.

### AID 1442

Cells (wild-type or mutant) were plated and allowed to settle at 37° C. overnight. The cells were then incubated with different concentrations of compound (with the final DMSO concentration at 0.25%) at 37° C. for 2 hours. SteadyLite luciferase reagent was added, plates were incubated for 30 minutes and then read on Envision reader.

First, 384 well plates were filled with 24 μl of cells (wild-type or mutant) in DMEM (7500 cells per well) using Wellmate (all columns except 1 and 23). 24 μl DMEM were added to columns 1 and 23 using Wellmate. Then, plates were sealed with Breathe-easy membranes and incubated at 37° C. overnight. 1 μl of compound was diluted from the dose-response plate with 79 μl DMEM to get a diluted dose-response plate (top concentration 125 μM). 6 μl of diluted compound was added to the overnight-grown cells, and the mixture was sealed with Breathe-easy membrane and incubated at 37° C. for 2 hours. Then, 30 μl SteadyLite HTS reagent was added, and the mixture was incubated at room temperature for 30 minutes. Luminescence was read on Envision reader.

The data was analyzed in IDBS ActivityBase. Each dose-response plate contained compounds in columns 3-22, controls (wild-type or mutant cells, no compound) in columns 2 and 24, and blanks (no cells) in columns 1 and 23. Each column 3-22 contained 16 two-fold dilutions of a single compound, ranging in concentration from 25 μM to 0.75 nM. Percent enhancement of signal was calculated for each well from the signal in luminescence units (LU) and the mean of the plate controls and the mean of the plate blanks using the following equation:

% enhancement=100*(((signal−blank mean)−(control mean−blank mean))/(control mean−blank mean)))

Dose response curves of percent enhancement were fit using XLfit equation 205 (four parameter logistic model).

IC₅₀ scores were calculated based on the maximum percent enhancement and the EC₅₀ value as follows:
(1) For EC₅₀>0, Score=maximum percent enhancement/2
(2) For EC₅₀=0 (bad curve fit), Score=0

Maximum percent enhancement shown by each compound is reported and is used to determine potency of the compound along with the EC₅₀ value.

Activity outcome is reported as follows:
(1) EC₅₀>0 and maximum percent enhancement>22=Active
(2) EC₅₀>0 and maximum percent enhancement<22=Inactive
   Hence, active compounds have a score greater than 11, and inactive compounds have a score of less than 11. Of the 40 compounds tested, 32 were active, and 8 were inactive.

### AID 1444

Cells (wild-type or mutant) were plated and allowed to settle at 37° C. overnight. The cells were then incubated with different concentrations of compound (final DMSO concentration: 0.25%) at 37° C. for 2 hours. SteadyLite luciferase reagent was added, plates incubated for 30 minutes and then read on Envision reader.

384 well plates were filled with 24 μl of cells (wild-type or mutant) in DMEM (7500 cells per well) using Wellmate (all columns except 1 and 23). Then, 24 μl DMEM were added to columns 1 and 23 using Wellmate. Plates were sealed with Breathe-easy membranes and incubated at 37° C. overnight. 1 μl of compound was diluted with 79 μl DMEM to get a diluted dose-response plate (top concentration 125 μM). 6 μl of diluted compound was added to the overnight-grown cells. The mixture was sealed with Breathe-easy membrane and incubated at 37° C. for 2 hours. 30 μl SteadyLite FITS reagent was then added, and the mixture was incubated at room temperature for 30 minutes. Luminescence was read on Envision reader.

The data was analyzed in IDBS ActivityBase. Each dose-response plate contained compounds in columns 3-22, controls (wild-type or mutant cells, no compound) in columns 2 and 24, and blanks (no cells) in columns 1 and 23. Each column 3-22 contained 16 two-fold dilutions of a single compound, ranging in concentration from 25 μM to 0.75 nM. Percent enhancement of signal was calculated for each well from the signal in luminescence units (LU) and the mean of the plate controls and the mean of the plate blanks using the following equation:

% enhancement=100*(((signal−blank mean)−(control mean−blank mean))/(control mean−blank mean)))

Dose response curves of percent enhancement were fit using XLfit equation 205 (four parameter logistic model).

IC₅₀ scores were calculated based on the maximum percent enhancement and the EC₅₀ value as follows:
(1) For EC₅₀>0, Score=maximum percent enhancement/2
(2) For EC₅₀=0 (bad curve fit), Score=0

Maximum percent enhancement shown by each compound is reported and is used to determine potency of the compound along with the EC₅₀ value.

Activity outcome was reported as follows:
(1) EC₅₀>0 and maximum percent enhancement>30=Active
(2) EC₅₀>0 and maximum percent enhancement<30=Inactive
   Hence, active compounds have a score of greater than 15, and inactive compounds have a score of less than 15.

Forty compounds were tested, and all of them were active.

### Western Blot Analysis

Cells were seeded and treated with the indicated compound(s). Cells were lysed after each time point in a buffer of 50 mM TrisHCl pH 7.5, 200 mM NaCl, 0.5% NP-40, and 1 complete mini EDTA-free protease inhibitor cocktail tablet per 10 mL of buffer (Roche, Cat. No. 8360170). Protein content was quantified using a Biorad protein assay reagent (Bio-Rad Laboratories, Hercules, Calif., Cat. No. 500-0006). SDS sample buffer was added to the samples, and the mixture was boiled for 5 minutes. Equal amounts of protein were resolved on a 1D SDS-PAGE gel. Following separation proteins were transferred to nitrocellulose membrane, blocked with 5% milk, and then incubated with primary antibody overnight at 4° C. The membrane was incubated with secondary-HRP antibody for 45 minutes at room temperature and developed with SuperSignal West Pico Substrate (Pierce, Rockford, Ill., Cat. No. 34080).

Immunoprecipitation experiments were performed with HA-affinity matrix (Roche, Cat. No. 11815016001) according to manufacturer's protocol. Lysates were immunoprecipitated for 3 hours and then washed 3 times with lyses buffer.

### In Vitro Ubiquitination Assay

Full-length Mdm2 in vitro ubiquitination reactions were performed in 15 μl reaction mixtures containing 50 mM TrisHC1 pH 7.5, 5 mM MgCl₂, 2 mM NaF, 2 mM ATP, 0.6 mM DTT, 50 ng E1 (Boston Biochem, Cat. No. E-305), 1 μg PK-ubiquitin, 50 ng His-UbCH5C, and 100 ng Flag-Mdm2. 50 ng His-p53 was used for in vitro p53 ubiquitination assay. 100 ng Flag-Mdm2 and 100 ng Ha-MdmX were used for ubiquitination reactions with the Mdm2-MdmX complex. After incubation for 30 minutes at 37° C., SDS sample buffer was added to the samples and they were boiled for 5 minutes. The products were resolved by SDS-PAGE and analyzed by Western blot with anti-p53, anti-Mdm2 SMP14, 2A10, 3G5 mix, or anti-MdmX antibodies.

Radiolabeled in vitro ubiquitination assays were carried out in 30 μl reaction mixtures with ³²P-labeled ubiquitin. 500 ng GST-Mdm2 (residues 400-491) and GST-MdmX (residues 410-490) were used for the RING-RING reactions. The products of the reaction were resolved on a SDS-PAGE gel and visualized by autoradiography.

Roc1/Cul1 protein was a kind gift from Zhen-Qiang Pan at the Mount Sinai School of Medicine. 3 μg of protein was used per ubiquitination reaction.

### Plasmids

Luciferase and Mdm2 or luciferase and Mdm2(C464A) were cloned into the NheI and XhoI sites on the pcDNA3.1 plasmid (Invitrogen, Cat. No. P/N 35-0574). The two genes were ligated into a fusion protein utilizing a BamH1 site on the C-terminus of luciferase and N-terminus of Mdm2. A linker (Gly Ser Gly Gly Gly Ser Gly Gly Gly Gly Ser Gly Gly Gly Gly Ser Gly Gly Thr Gly Ser, SEQ ID NO: 1) was cloned in between luciferase and Mdm2. DNA encoding the linker is listed as SEQ ID NO: 2 The primers used for cloning are listed below in Table 1.

TABLE 1

| | | SEQ |
|---|---|---|
| | | ID |
| Name | Sequence | NO. |
| Luciferase | GCT ACT GCT AGC C ACC ATG GAA GAC | 3 |
| Forward | GCC AAA AAC ATA AAG AAA GGC CCG | |
| | GCG CC | |
| Luciferase | AGT AGC AGT AGC GGA TCC CAC GGC | 4 |
| Reverse | GAT CTT TCC GCC CTT CTT GGC C | |
| MDM2 | GCT ACT GCT ACT GGA TCC GGA GGC | 5 |
| forward | GGA AGT GGT GGA GGC GGA AGC GGC | |
| | GGT GGA GGT AGT GGT GGA ACC GGT | |
| | TGC AAT ACC AAC ATG TCT GTA CCT | |
| | ACT GAT GGT GCT GTA ACC ACC TCA | |
| | CAG | |
| MDM2 | GCT ACT CTC GAG CTA GGG GAA ATA | 6 |
| Reverse | AGT TAG CAC AAT CAT TTG AAT TGG | |

### Additional Cell Lines

MCF10a (mammary breast fibrocystic disease), and BJEH (fibroblasts) cells were maintained in Dulbecco's modified Eagle's medium (DMEM) (Fisher Scientific, Cat. No. MT-15-018-CM) with 10% fetal bovine serum (FB) and 100 μg/ml penicillin/streptomycin (P/S). BJEH cells were a kind gift of Robert A. Weinberg at the Massachusetts Institute of Technology.

### Alamar Blue Viability Assay

Cells were seeded in 384-well black clear-bottom plates (Corning Inc., Corning, N.Y., Cat. No. 07-200-655). Cells were then treated with the indicated concentration of compound. Plates were incubated for 48 hours at 37° C. Alamar Blue (Invitrogen, Cat. No. DAL1100) was added to each well to a final concentration of 10% and incubated at 37° C. for 16 hours. Fluorescence intensity was determined using the Victor3 Plate Reader (PerkinElmer). Median percent survival or growth inhibition was calculated and normalized to a no treatment control.

### Bliss Independence Model

Bliss independence was determined using the formula C=A+B−A*B. C is the combined response for the two single compounds with effects A and B. A is the percent growth inhibition with compound A at a particular concentration and B is the percent growth inhibition with compound B at a particular concentration. The excess over the predicted Bliss independence was determined by subtracting the predicted Bliss effect for each treatment from the experimentally determined growth inhibition for the same treatment.

### Protein Purification

PK-ubiquitin purification, UbCH5c, GST-Mdm2 (400-491), and GST-MdmX (410-490) has been previously described (35). p53 cloned with a 6×His-Tag at the N-terminus of the proteins, were expressed in BL21 E. coli cells and purified using Ni-NTA Agarose (Qiagen, Cat. No. 30210) according to the manufacturer's protocols.

Insect Sf9 cells were infected with recombinant viruses encoding Flag-tagged Mdm2 and HA-tagged MdmX, either individually or together. Cells were lysed by sonication and soluble extracts bound to M2-Agarose (Sigma, Cat. No. A2220) in buffer A (50 mM TrisCl pH 8.0, 150 mM NaCl, 10% glycerol and protease inhibitors). Following extensive washing with buffer A containing 300 mM NaCl, proteins were eluted with 1 mg/ml Flag-peptide (Sigma, Cat. No. F3290) in buffer A. For complex purification Flag-peptide eluted material was bound to HA-agarose (Roche, Cat. No. 11815016001) in buffer A and eluted with 1 mg/ml HA-peptide at 30° C.

RNA Extraction and qRT-PCR Analysis

RNA was harvested using RNeasy Mini kit (Qiagen, Cat. No. 74104) and first-strand cDNA synthesis was performed with the TaqMan Reverse Transcription Reagents for qRT-PCR (Applied Biosystems Inc., Foster City, Calif., Cat. No. N8080234) according to the manufacturers' protocol. Samples were analyzed by quantitative real-time PCR on an ABI 7300 real-time PCR instrument (Applied Biosystems) using the ΔΔCT method. PCR reaction mixtures contained 75 ng of cDNA, 1×SYBR Green Mix (Applied Biosystems, Cat. No. 4367659), and 100 nM primers. Values were normalized to those of the hprt1 housekeeping gene (47).

TABLE 2
qRT-PCR primers

| Name | Sequence | SEQ ID NO: |
|---|---|---|
| Mdm2 Forward | TTGGCGTGCCAAGCTTCTCT | 7 |
| Mdm2 Reverse | TACCTGAGTCCGATGATTCC | 8 |
| p21 Forward | GGCGGCAGACCAGCATGACAGATT | 9 |
| p21 Reverse | GCAGGGGGCGGCCAGGGTAT | 10 |
| Puma Forward | CCTGGAGGGTCCTGTACAATCT | 11 |
| Puma Reverse | GCACCTAATTGGGCTCCATCT | 12 |
| Bax Forward | TGGAGCTGCAGAGGATGATTG | 13 |
| Bax Reverse | AAACATGTCAGCTGCCACTCG | 14 |
| Hrpt1 Forward | TGACACTGGCAAAACAATGCA | 15 |
| Hrpt1 Reverse | GGTCCTTTTCACCAGCAAGCT | 16 |

### MEL23

The ¹H-NMR (500 MHz, CDCl₃) of MEL23 is as follows: δ p.p.m. 10.51 (s, 1H), 9.65 (s, 1H), 9.11 (s, 1H), 8.48 (s, 1H), 7.38-7.36 (m, 1H), 7.28-7.26 (m, 1H), 7.02-6.93 (m, 2H), 5.63 (s, 1H), 3.66-3.58 (m, 3H), 3.31 (m, 1H), 2.96 (m, 1H), 2.83 (m, 1H), 1.45-1.40 (m, 2H), 1.27-1.20 (m, 2H), 0.87-0.84 (m, 3H).

The ¹³C-NMR (300 MHz, CDCl₃) of MEL23 is as follows: δ p.p.m. 164.35, 163.75, 153.05, 136.85, 133.52, 127.24, 121.49, 119.28, 118.26, 112.26, 105.31, 80.34, 51.59, 43.54, 39.31, 31.30, 20.67, 19.43, 14.68.

The high resolution magnetic sector data (HRMS) (m/z) of MEL23 is as follows: [M]⁺ calculated for C₁₉H₂₃O₃N₄, 355.1770. found, 355.1775.

### MEL24 (Re-Synthesis)

The ¹H-NMR (500 MHz, CDCl₃) of MEL24 is as follows: δ p.p.m. 10.52 (s, 1H), 7.38-7.37 (m, 1H), 7.25-7.24 (m, 1H), 7.02-6.94 (m, 2H), 5.70 (s, 1H), 3.61-3.57 (m, 1H), 3.38-3.33 (m, 1H), 3.09 (s, 6H), 3.03-2.88 (m, 1H), 2.87-2.84 (m, 1H).

The ¹³C-NMR (300 MHz, CDCl₃) of MEL24 is as follows: δ p.p.m. 163.09 (2C), 153.71, 136.80, 133.43, 127.25, 121.50, 119.30, 118.28, 112.20, 105.41, 80.58, 51.96, 43.53, 27.64 (2C), 19.43.

The high resolution magnetic sector data (HRMS) (m/z) of MEL24 is as follows: [M]⁺ calculated for C₁₇H₁₉O₃N₄, 327.1457. found, 327.1479.

### Example 2

### High-Throughput Screening in a Cell-Based Mdm2-Stability Assay

To identify Mdm2 E3 ligase inhibitors, a high-throughput cellular assay to measure changes in Mdm2 auto-ubiquitination and degradation was designed. This assay took advantage of the fact that Mdm2 can regulate itself in cells through ubiquitination and degradation (8). Two fusion proteins were constructed for the assay: (a) a wild-type Mdm2-luciferase fusion protein, which can auto-ubiquitinate and thereby target itself for degradation; and (b) a mutant Mdm2(C464A)-luciferase fusion protein. Both fusion proteins were constructed with luciferase N-terminal to Mdm2. The C464A mutation disrupts a metal binding site in the RING domain, thereby preventing Mdm2 E3 ligase activity (8). Small molecules that increase the luminescence of the Mdm2-luciferase fusion protein without increasing the luminescence of the Mdm2(C464A)-luciferase fusion protein are likely inhibiting Mdm2 E3 ligase activity or the proteasomal degradation of Mdm2. Alternatively, small molecules that increase the luminescence of both fusion proteins are likely affecting transcription, translation, or have a general cellular impact that is not dependent on the E3 ligase activity of Mdm2 or proteasomal degradation.

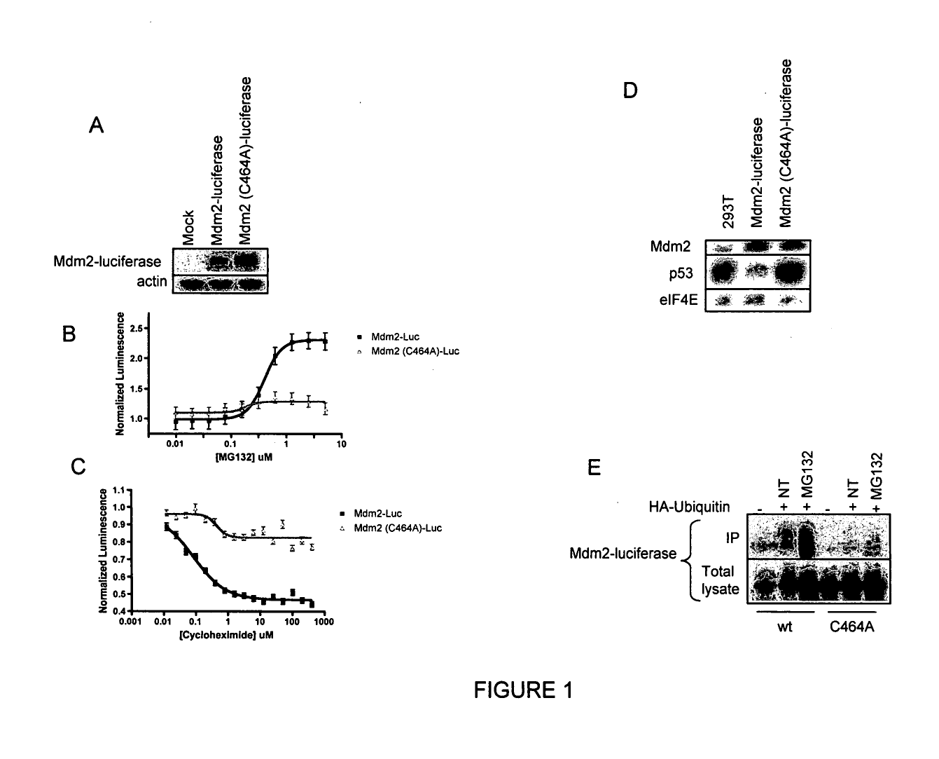

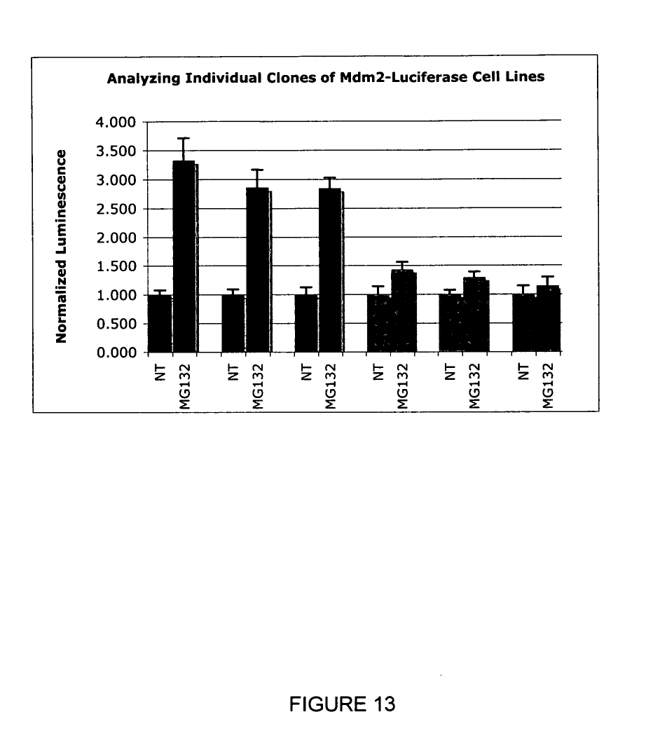

The expression levels of the two fusion proteins in 293T cells were compared. Consistent with auto-ubiquitination, Mdm2-luciferase protein was expressed at lower levels than the mutant Mdm2(C464A)-luciferase protein, because the wild type protein is an active ligase that can auto-degrade (FIG. 1A). To confirm that the difference in luminescence levels between the two constructs was the result of a difference in degradation rates, stably-expressing cell lines in 293T cells were made. They were treated with a proteasome inhibitor, MG132 (Z-Leu-Leu-Leu-aldehyde). Proteasome inhibition increased the levels of the Mdm2-luciferase protein, but not the Mdm2(C646A)-luciferase mutant, in multiple clonal cell lines (FIG. 1B and FIG. 13). Treatment with cycloheximide selectively decreased the luminescence of the Mdm2-luciferase protein, suggesting that the observed decrease in stability was due to a post-translational mechanism (FIG. 1C).

To examine the ubiquitin ligase activity of the Mdm2-luciferase construct, wild-type and mutant Mdm2-luciferase cell lines were transfected with HA-ubiquitin and cell lysates were immunoprecipitated with an anti-HA antibody. The Mdm2-luciferase protein immunoprecipitated with the anti-HA affinity matrix, indicating that it is ubiquitinated in cells, whereas the Mdm2(C464A)-luciferase protein immunoprecipitated to a significantly lesser extent (FIG. 1E). Furthermore, the Mdm2-luciferase cell line had overall lower endogenous p53 levels compared to the Mdm2(C464A)-luciferase cell line, suggesting that the Mdm2-luciferase fusion protein was able to regulate endogenous p53 (FIG. 1D). Together, these results confirmed that Mdm2-luciferase is rapidly degraded after auto-ubiquitination through the proteasome, and its stability is regulated by an intact RING domain.

The above experiments verified that the cell lines could function as a valid system to screen for Mdm2 E3 ligase inhibitors. Next, 270,080 compounds were screened: 51,356 compounds were screened at 5.33 μg/ml in a 384-well format, and 218,724 compounds were screened at 5 μM in 1536-well format (MLSCN library, AID 1442, 1230, 1444, and 1394). All compounds were incubated with the Mdm2-luciferase cell line for 2 hours, after which relative luminescence was detected by the addition of a luminesence buffer. The time point of 2 hours was chosen, as it is sufficient to see increases in Mdm2 due to its short half life of −20 minutes (66) and to minimize secondary effects that could affect luminescence levels. Compounds that increased luminescence in the primary screen were tested in a counter screen in the mutant Mdm2(C464A)-luciferase cell line to eliminate compounds with effects not dependent on Mdm2 degradation. A total of 57 primary hits were found. A series of secondary assays were performed to validate the hits. The first of these was to re-order the hit compounds and test them in a dilution curve in both Mdm2-luciferase cell lines. The effects of the compounds on endogenous p53 and Mdm2 levels and Mdm2 ubiquitination activity were then tested in multiple cell lines. Only two analogs, Mdm2 E3 Ligase Inhibitor 23 and 24 (MEL23 and MEL24), were ultimately validated to be effective Mdm2 inhibitors.

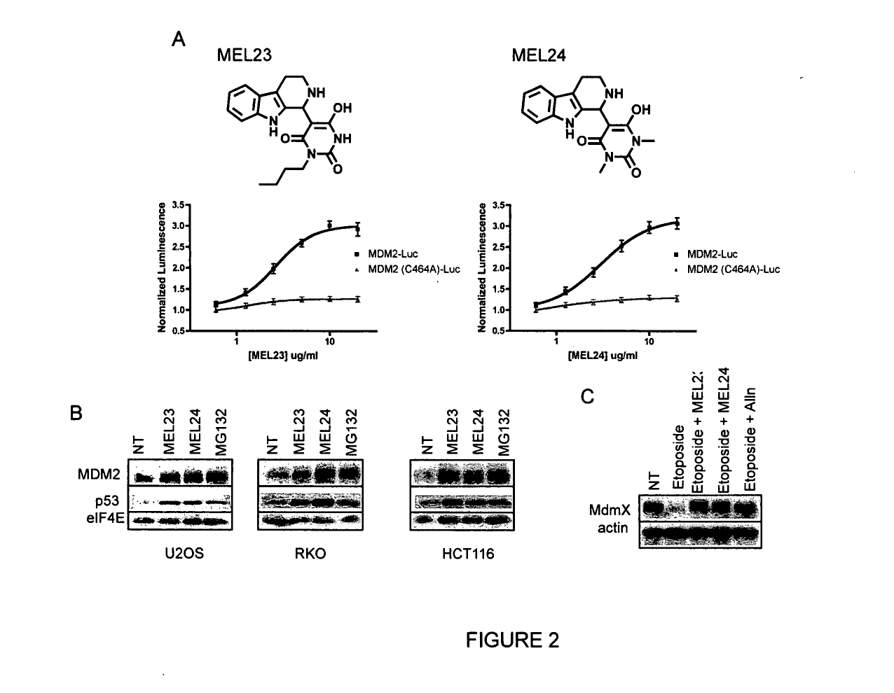

MEL23 and MEL24 increased the luminescence of the Mdm2-luciferase cell line to a similar level as with MG132 treatment and did not significantly affect the luminescence of the control Mdm2(C464A)-luciferase cell line (FIG. 2A). The EC₅₀ values of MEL23 and MEL24 were determined to be 2.7 μg/ml and 3.0 μg/ml (7.5 μM and 9.2 μM) respectively.

Using full-length Mdm2 allowed for the presence of all domains of Mdm2 in order to improve the likelihood of discovering inhibitors. Additionally, the use of a cell-based assay enabled targeting of potential cellular co-factors necessary for ligase activity. The high-throughput screen presented here could be modified in order to discover inhibitors of other E3 ligases. The ease of this type of high-throughput screen and its adaptability may therefore be valuable in the identification of new small molecule inhibitors directed against different E3 ligases.

### Example 3

### The Structure of the MEL Compounds and their Effects on Mdm2, p53, and MDMX in Wild-Type p53 Cell Lines

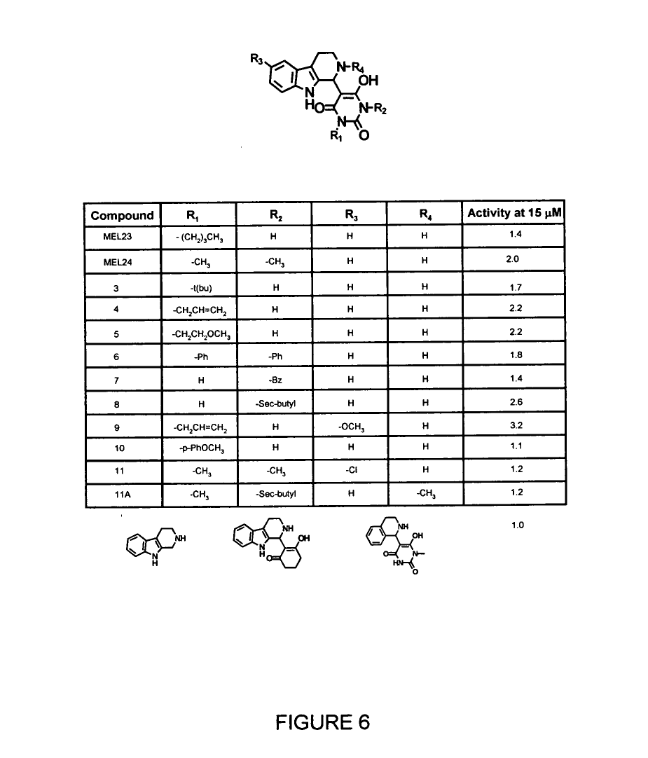

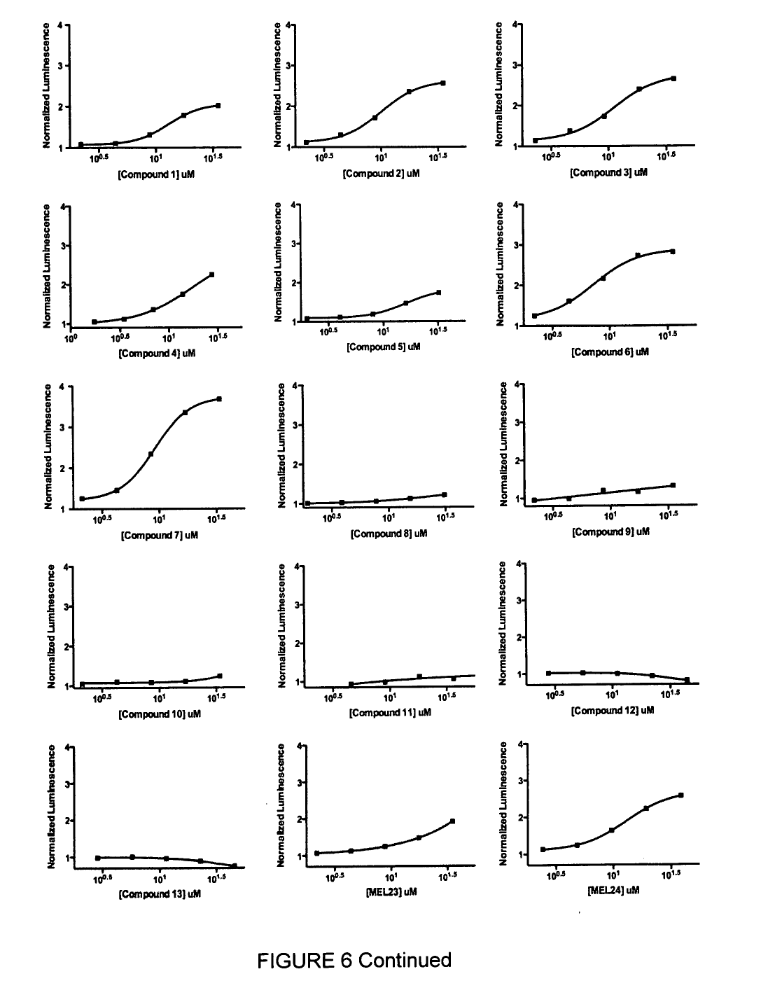

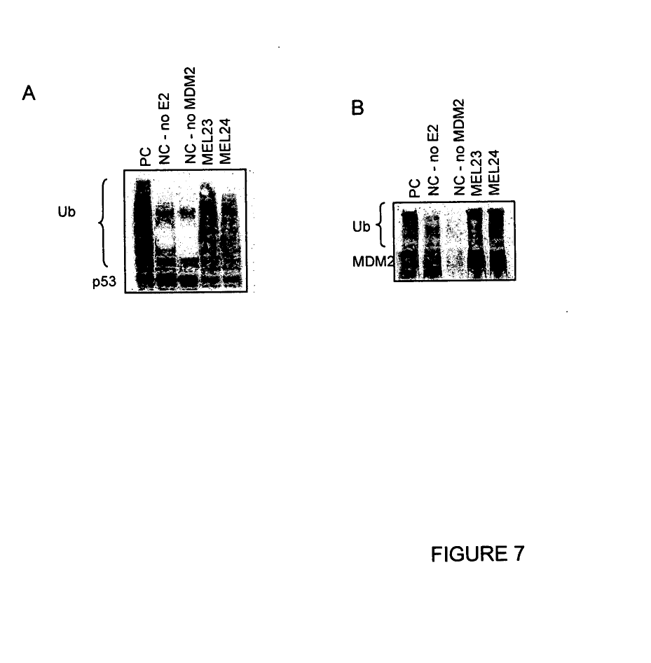

The MEL compounds are composed of tetrahydro-beta-carboline and barbituric acid moieties with slightly different substituents. Testing a series of analogs revealed that both the tetrahydro-beta-carboline and barbituric acid moieties are necessary for activity. However, addition of different substituents to these basic scaffolds could increase, decrease, or eliminate activity (FIG. 6).

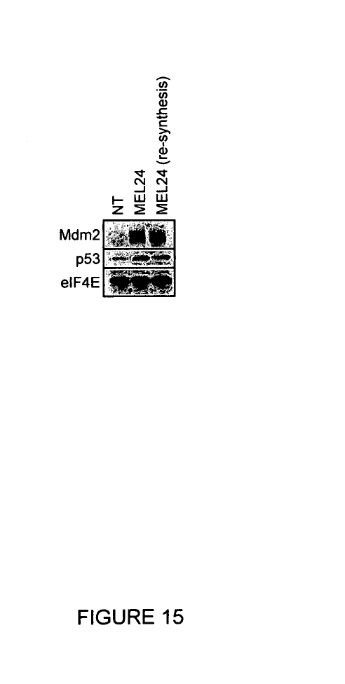

An Mdm2 E3 ligase inhibitor should prevent the degradation of Mdm2 substrates, including Mdm2, p53, and MdmX. Accordingly, Mdm2 and p53 were increased in three wild-type p53 cell lines (U2OS, RKO, HCT116) following incubation with 5 μg/ml MEL23 or MEL24 for 6 hours to similar levels as after MG132 treatment (FIGS. 2B, 3B, and 15). MdmX is stable in unstressed cells, but upon DNA damage signals, MdmX is rapidly degraded by Mdm2 E3 ligase activity (67). Accordingly, when MCF7 cells were treated with etoposide (30 μg/ml), MdmX levels decreased substantially. This degradation could be prevented when cells were co-treated with 50 μl ALLN (N-Acetyl-Leu-Leu-Nle-CHO), a proteasome inhibitor, or 5 μg/ml MEL23 or MEL24 for 16 hours (FIG. 2C). Thus, MEL compounds inhibit endogenous Mdm2 in multiple cell lines.

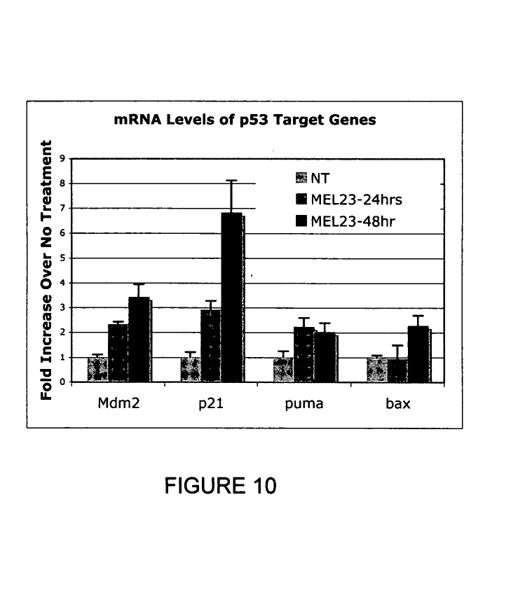

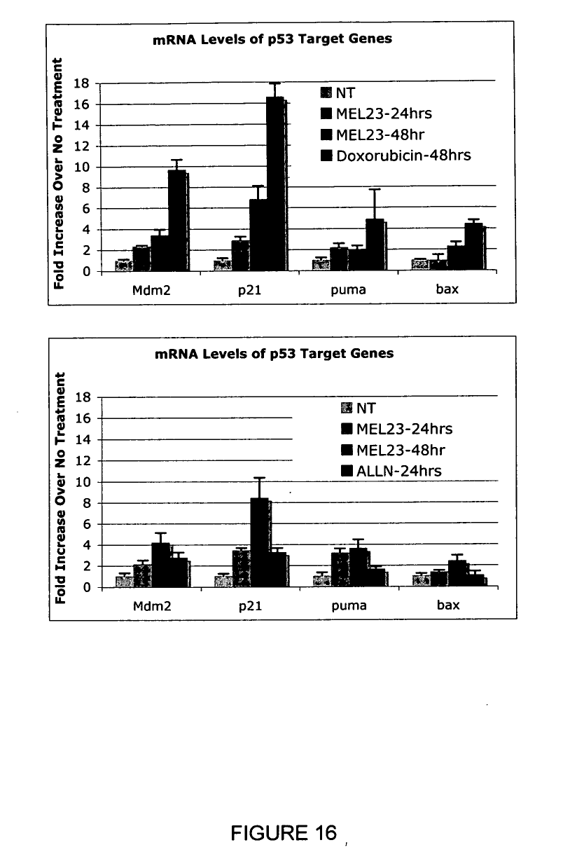

To determine if the increases in p53 protein levels led to a corresponding increase in the transcription of p53 target genes, several p53 target genes were analyzed by quantitative real-time PCR. In RKO cells treated with 5 μg/ml MEL23, mRNA levels of Mdm2, p21, Bax, and puma, all established p53 target genes (68), increased within 48 hours of treatment (FIG. 10). This increase in transcription was less than that seen with doxorubicin treatment, a DNA-damaging and p53-activating agent, but more than with ALLN (FIG. 16). Therefore, MEL compounds activate transcription of at least a certain set of p53 target genes.

### Example 4

### The Activity of MEL23 and MEL24 is Mdm2-Specific and Independent of p53 Transcription

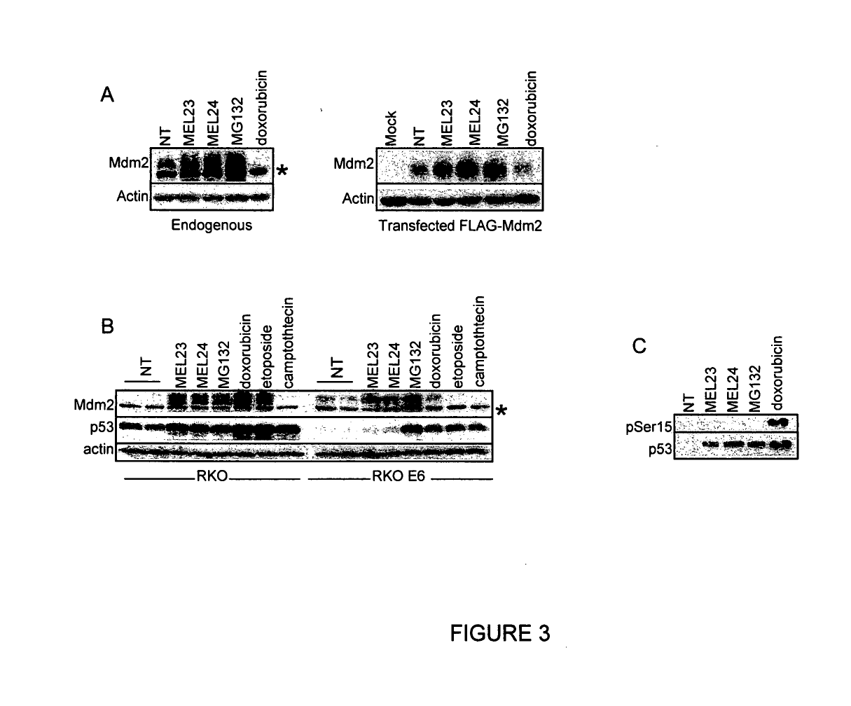

Many types of compounds lead to increases in both Mdm2 and p53 protein levels by inducing a DNA damage response. A series of experiments were performed to exclude the possibility that the MEL compounds were inducing p53-mediated stress, thereby leading to the accumulation of p53 and Mdm2. First, p53-null H1299 cells were treated with 5 μg/ml MEL23 or MEL24 for 6 hours. The levels of both endogenous Mdm2 protein and ectopically expressed Mdm2 increased after treatment, compared to no effect on Mdm2 levels with doxorubicin treatment (FIG. 3A). This indicates that the MEL compounds stabilize Mdm2 in a p53-independent manner. Further verification that MEL23 and MEL24 were not inducing a p53-stabilizing stress response was revealed by the lack of phosphorylation of p53 at serine-15 with MEL23 or MEL24 (5 μg/ml); a phosphorylation strongly induced by doxorubicin treatment (FIG. 3C).

The specificity of MEL23 and MEL24 for Mdm2 was tested in two isogenic cell lines, RKO and RKO-E6. RKO and RKO-E6 cells have wild-type p53 and Mdm2, but RK0-E6 cells express the HPV-E6 protein. The HPV-E6 protein forms an active E3 ligase in association with E6AP, a HECT domain E3 ligase, and targets p53 for degradation (69). As a result, p53 levels are significantly reduced in RKO-E6 cells compared to RKO cells (FIG. 3B, NT lanes). A compound that specifically inhibits Mdm2 E3 ligase activity should increase p53 levels in RKO cells, but not RKO-E6 cells. Consistently, treatment with 5 μg/ml MEL23 or MEL24 for 6 hours only increased levels of p53 in RKO cells, while treatments with MG132, doxorubicin, etoposide, and camptothecin, increased p53 in both cell lines through proteasome inhibition or though the activation of a DNA damage response (FIG. 3B). These data demonstrate that MEL23 and MEL24 do not inhibit the proteasome, do not inhibit E6AP activity, and do not induce a DNA damage response, confirming that MEL23 and MEL24 stabilize Mdm2 via a mechanism independent of p53 transcription.

### Example 5

### MEL23 and MEL24 Inhibit Mdm2-MdmX E3 Ligase Activity and p53 Ubiquitination in Cells

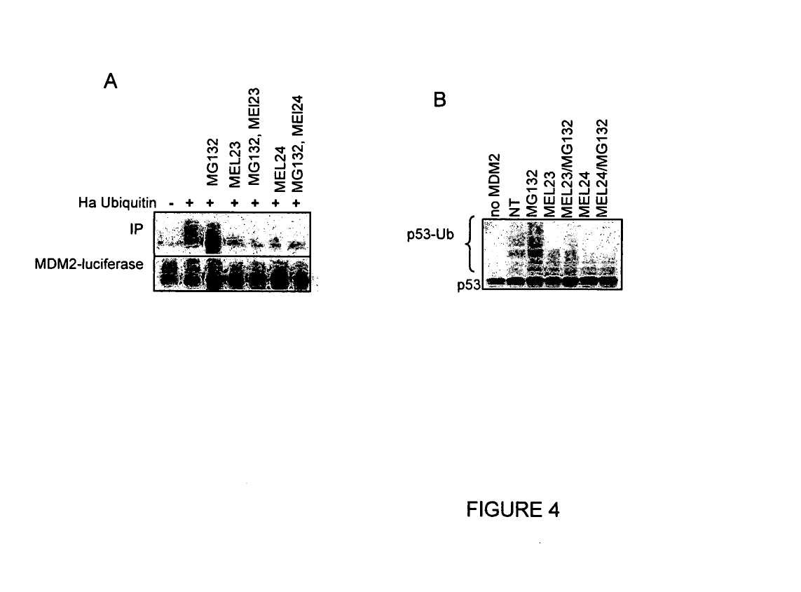

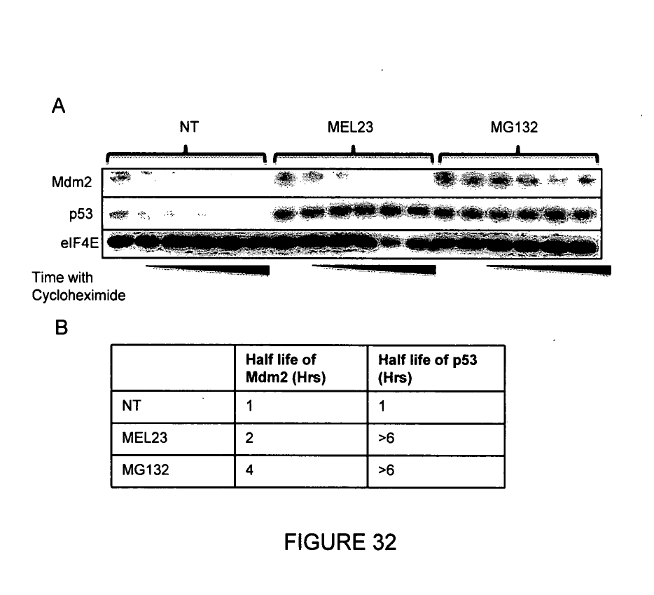

To determine whether the MEL compounds inhibit Mdm2 and p53 ubiquitination in cells, Mdm2 and p53 ubiquitin conjugates were analyzed in cell-based ubiquitination assays. The Mdm2-luciferase cell line was transfected with HA-ubiquitin and pre-treated with 10 μg/ml MEL23 or MEL24, followed by 10 μM MG132. An increased concentration of the compounds, 10 μg/ml, was used to facilitate complete inhibition of ubiquitination. Cells lysates were immunoprecipitated with an anti-HA antibody and analyzed by Western blotting. In cells treated with MEL23 or MEL24, significantly less Mdm2-ubiquitin conjugates were immunoprecipitated, indicating that the MEL compounds inhibit ubiquitination of Mdm2 (FIG. 4A). The inhibition of Mdm2 ubiquitination led to a stabilization of Mdm2 protein, as indicated by its half-life increasing from one hour to 2 hours with 5 μg/ml MEL23 treatment (FIG. 32).

To test the effects of the MEL compounds on p53 ubiquitination, H1299 cells were transfected with plasmids expressing p53, Mdm2, and ubiquitin and pre-treated with 10 μg/ml MEL23 or MEL24, followed by treatment with 10 μM MG132. Ubiquitination of p53 was analyzed by Western blotting. Cells treated with MG132 showed a significant increase in p53 ubiquitination that was eliminated by pre-treatment with MEL23 or MEL24 (FIG. 4B). Additionally, treatment of MEL23 or MEL24 alone demonstrated that the compounds stabilized p53 without ubiquitination. In fact, the half-life of p53 increased from an hour to greater than 6 hours with 5 μg/ml MEL23 treatment (FIG. 32). Thus, treatment with MEL23 and MEL24 inhibits ubiquitin conjugation to Mdm2 and p53.

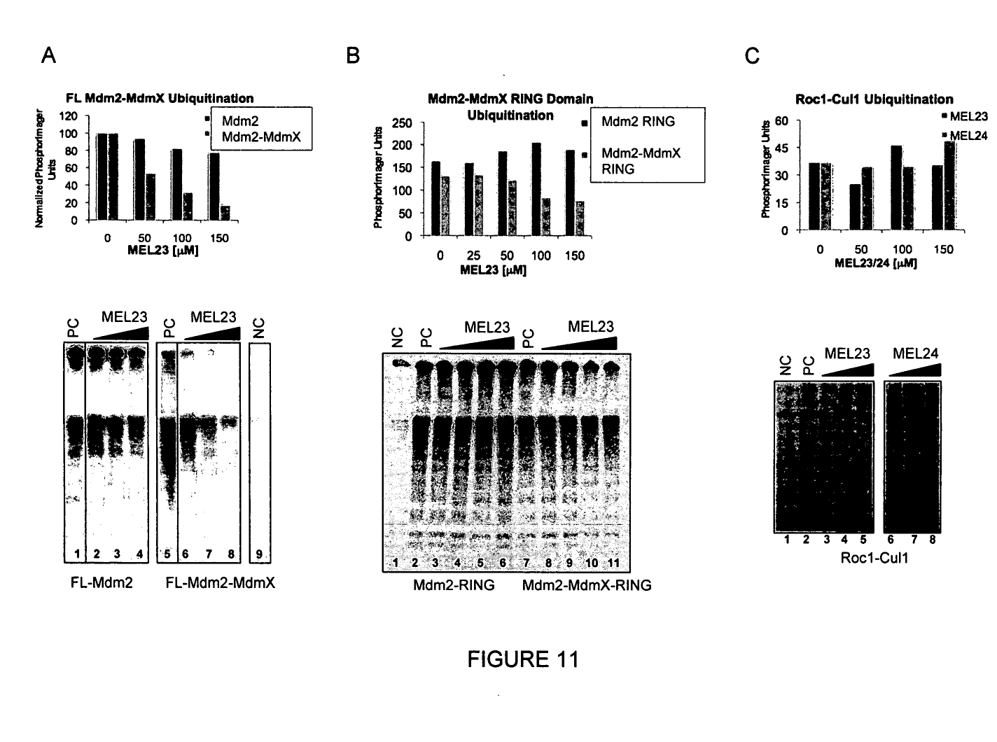

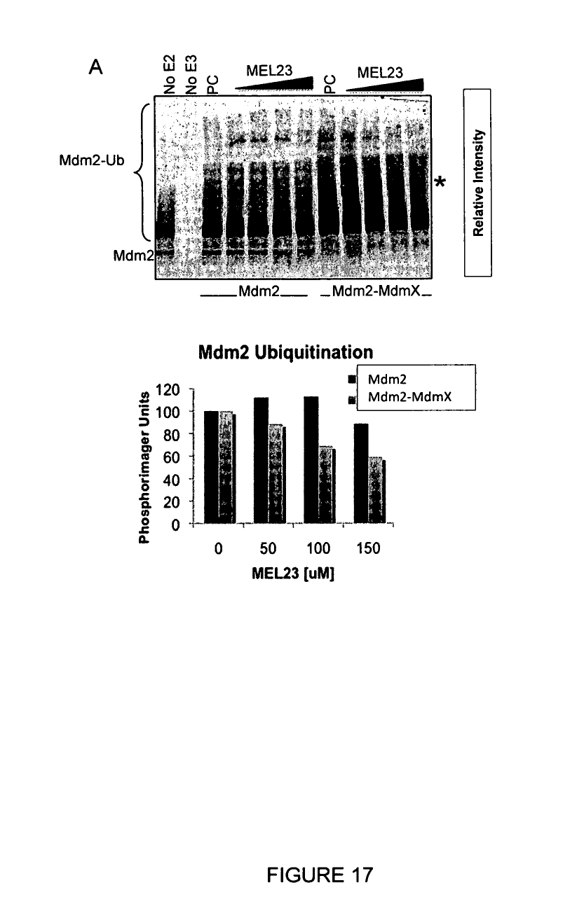

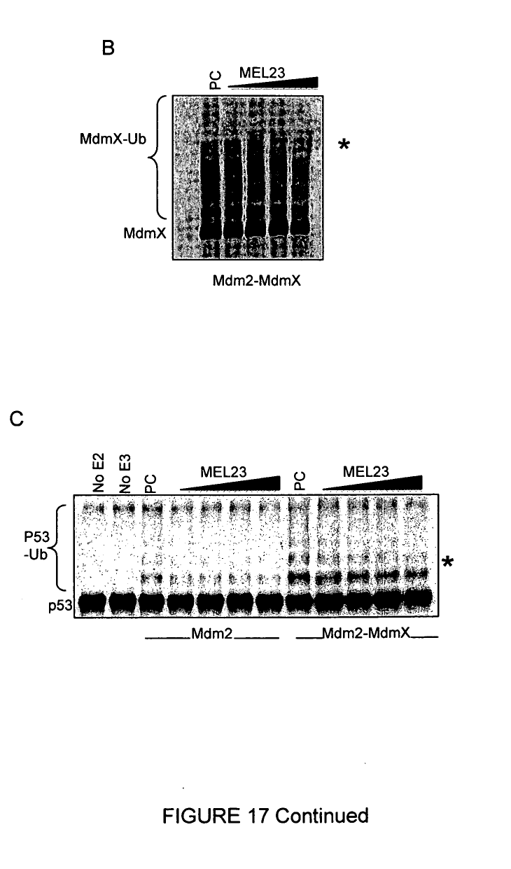

The specificity of the MEL compounds in in vitro ubiquitination reactions also was tested. This assay included purified E1, UbCH5C, ³²P-labeled ubiquitination, and ATP. However, the addition of increasing amounts of MEL23 in the ubiquitination reactions containing full-length Flag-Mdm2 had little to no inhibitory effect on the Mdm2 ligase activity (FIG. 11A). Because Mdm2 is able to make a functional complex with MdmX, and this complex is reported to have different activity and specificity in cells, the activity of MEL23 on the purified Flag-Mdm2-HA-MdmX complex was tested. MEL23 significantly decreased the overall ligase activity of the Mdm2-MdmX complex (FIG. 11A). Western blots against Mdm2 and MdmX ubiquitin conjugates, upon MEL23 treatment, also showed inhibition of the Mdm2-MdmX complex, albeit at higher concentrations of compounds (FIG. 17); this assay format may be less sensitive compared to radiolabel detection. MEL23 also inhibited the activity of the Mdm2-MdmX complex toward p53 (FIG. 17).

Mdm2 and MdmX interact through a binding surface within their RING domains (53); therefore, it was hypothesized that the target of the MEL compounds is the Mdm2 RING-MdmX RING domain interface. The activity of the compounds on the purified RING domains of the proteins was tested. The compounds inhibited the ubiquitination of the Mdm2 RING-MdmX RING hetero-complex but not that of the Mdm2 RING homo-complex (FIG. 11B). However, unlike the full-length complex, which was produced through co-expression of Mdm2 and MdmX in SF9 cells followed by two consecutive purification steps, the RING domain ubiquitination reactions were performed by mixing individually expressed and purified Mdm2 RING and MdmX RING proteins. Thus, it is likely that both active hetero- and homo-RING domain complexes were present in the reactions. Because only the Mdm2-MdmX hetero-complex was inhibited, the overall extent of the inhibition by MEL23 was lower than with the full-length hetero-complex.

In order to further test the specificity of the MEL compounds, the Roc1/Cul1 E3 RING domain ligase was analyzed. The Roc1/Cul1 E3 RING domain ligase is also a multi-subunit ligase (70), which showed no inhibition when tested with MEL23 and MEL24 (FIG. 11C). Of note here, the fact that MEL23 treatment had no effect on Mdm2 activity also provides an additional specificity control.

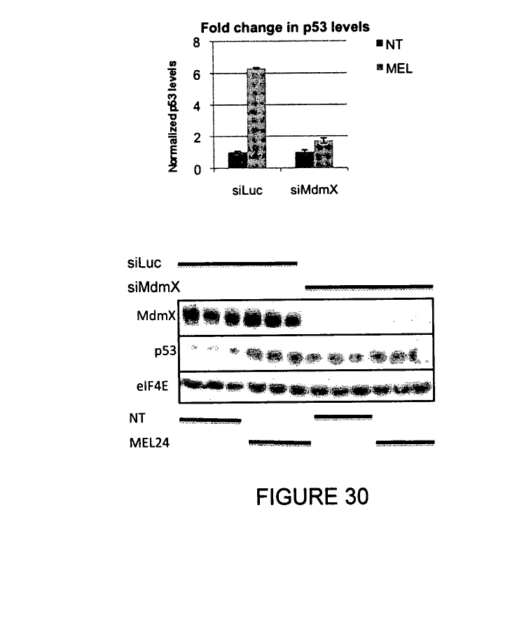

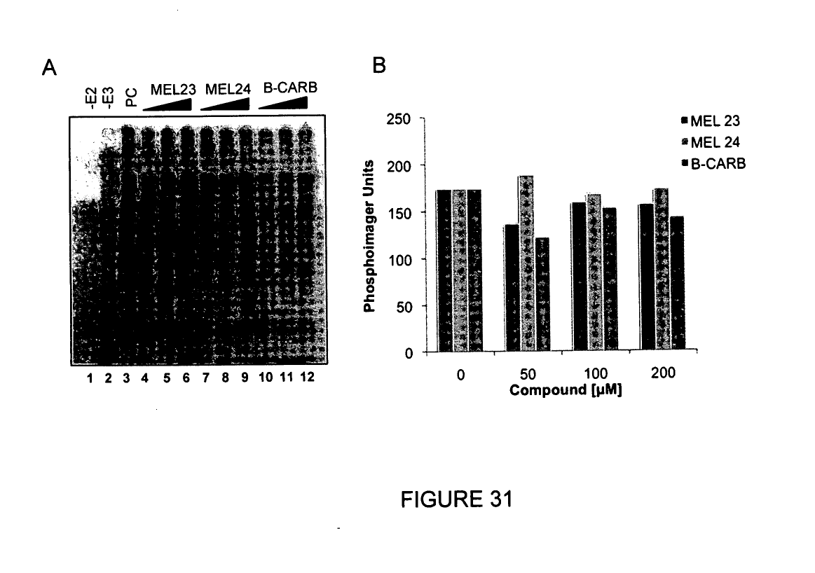

To confirm that the MEL compounds act on the Mdm2-Mdm2 complex in cells, the MdmX levels in the cell was decreased with siRNA. siMdmX transfected cells treated with MEL24 showed relatively stable p53 levels, less than a two-fold increase in p53 protein compared to non-treated cells (FIG. 30). In comparison, cells treated with MEL24 and a non-targeting siRNA showed an increase of greater than six-fold in p53 proteins levels compared to non-treated cells. This result demonstrates that without MdmX present in the cell the MEL compounds are inefficient in preventing p53 degradation, and therefore likely function on the Mdm2-MdmX complex.

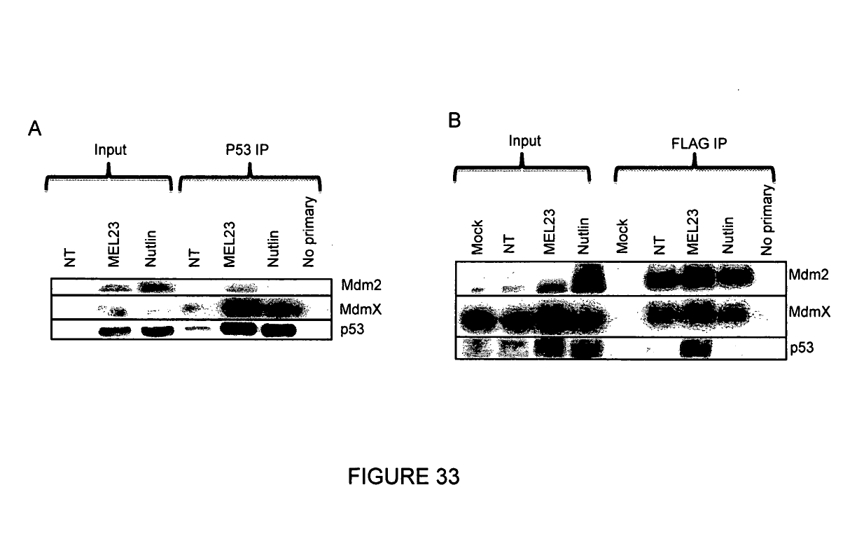

Although it was determined that the MEL compounds likely interfere with the Mdm2-MdmX E3 ligase activity, they were not found to inhibit complex formation between Mdm2 and MdmX with co-immunoprecipation (FIG. 33). Of note, MEL23 also did not inhibit p53-Mdm2 complex formation. Taken together, the results suggest that the E3 ligase activity of the Mdm2-MdmX hetero-complex is the primary molecular target of the MEL compounds in cells, although the compounds function in a mechanism other than interfering with the protein-protein interaction.

Mdm2 inhibitors described to date, include peptide inhibitors (71) and small molecules such as Nutlin-3 (13) and RITA (72). Mdm2 E3 ligase inhibitors identified by in vitro screens, such as the HLI series of compounds, have been described (15). Although the HLI compounds were able to induce p53-dependent apoptosis, they lack specificity towards Mdm2. Other Mdm2 E3 ligase inhibitors identified in in vitro ubiquitination assays, such as sempervirine and lissochlinidine B, have not been tested for specificity in cells (17, 19). More recently, the first small-molecule inhibitor of MdmX binding to p53 was identified (81). Unlike other Mdm2 and MdmX inhibitors identified by biochemical in vitro screening, MEL23 and MEL24 are a unique class of Mdm2 inhibitors identified in a cell-based assay that are able to specifically inhibit the E3 ligase activity of the Mdm2-MdmX complex.

The data demonstrating that MEL23 and MEL24 are selective in their ability to inhibit the E3 ligase activity of Mdm2-MdmX in vitro and stabilize p53 and Mdm2 in cells, indicate that the Mdm2-MdmX complex is centrally involved in regulating the degradation of these proteins. These observations are in agreement with multiple studies that describe the importance and activity of the Mdm2-MdmX complex. While in vitro Mdm2 can catalyze p53 ubiquitination (73), in cells MdmX is needed along with Mdm2 for efficient p53 degradation (55). MdmX can lower the concentration of Mdm2 needed for both p53 ubiquitination and auto-ubiquitination and the Mdm2-MdmX complex has been shown to be a better ligase for p53 than Mdm2 alone (54, 74-75). Enhancement of activity of RING domain hetero-oligomers has also been demonstrated for BCRA1-BARD1 complexes, where the presence of BARD1 in the complex increases the ligase activity of BCRA1 (76). Therefore these results support other evidence suggesting that although Mdm2 is able to function as an E3 ligase on its own, MdmX augments the nature and activity of the ligase with profound functional consequences in cells.

As specific Mdm2-MdmX complex inhibitors, MEL compounds may provide further insight into the function of the Mdm2-MdmX E3 ligase and allow for investigation of the differences in activity between the hetero-complex and the Mdm2 homo-complex. It will be highly informative to determine the precise biophysical mechanism of action of the MEL compounds, as well as the binding site of the compounds. Additionally, the MEL compounds may be used as molecular tools to validate novel targets of Mdm2. An improved understanding of the mechanism of action of the MEL compounds may lead to new ways to inhibit E3 ligases, which could be beneficial in diverse areas.

### Example 6

### MEL23 Leads to Cell Death that is p53-Dependent and Synergistic with DNA Damaging Agents

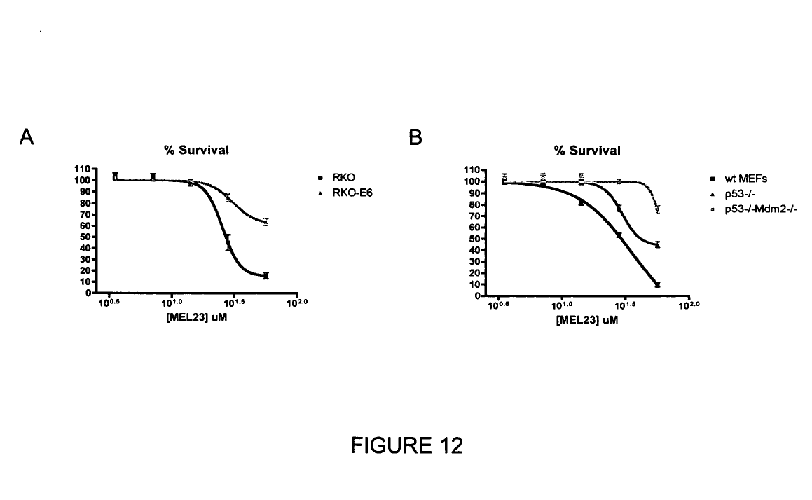

To test the physiological outcomes and potential therapeutic efficacy of the MEL compounds, the survival of RKO and RK0-E6 cells after treatment with MEL23 was examined. Because treatment of RKO cells with the MEL compounds led to an increase in p53 levels and activity (FIGS. 2B, 3B, and 10), a corresponding increase in p53-dependent cell death is expected. Consistent with this expectation, RKO cell survival decreased in a dose-dependent manner following MEL23 treatment, while RK0-E6 cells remained largely unaffected (FIG. 12A).

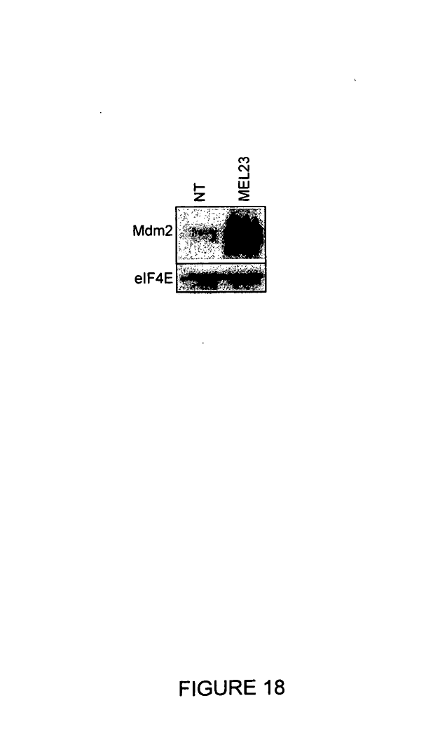

To further test p53-dependence and Mdm2-dependence of MEL23-induced cell death, three mouse embryonic fibroblasts (MEFs) cell lines, wild-type, p53^{-/-}, or p53^{-/-} mdm2^{-/-}, were analyzed (FIGS. 12B and 18). Consistently, the compounds showed p53-dependent activity, as the survival of the wild-type cells decreased the greatest with a dose-dependent increase in MEL23. However, the survival of the p53 MEFs was also decreased with MEL23 treatment, indicating some p53-independent, Mdm2-dependent activity. This is consistent with reports that Mdm2 has oncogenic effects independent of p53 (12). Only a small decrease in cell survival was seen with the p53^{-/-} mdm2^{-/-} MEFs at the highest concentration of MEL23 compound tested. Therefore, consistent with the Mdm2-MdmX complex being the target of the MEL compounds, the majority of growth inhibition was dependent on the presence of Mdm2.

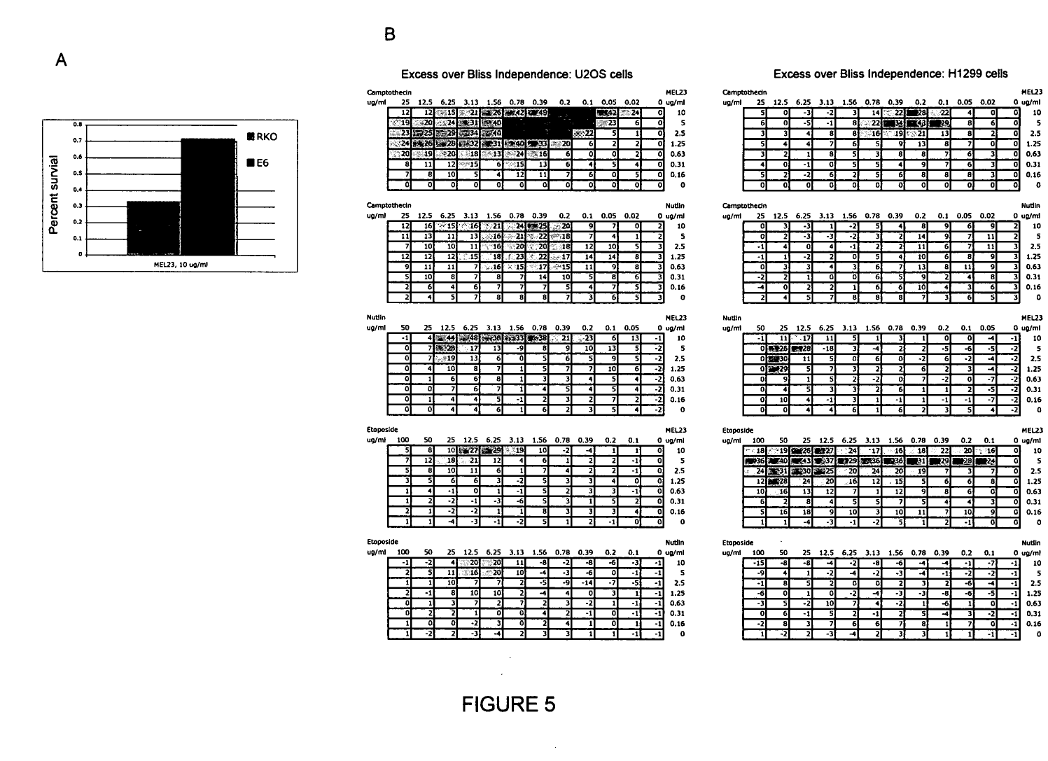

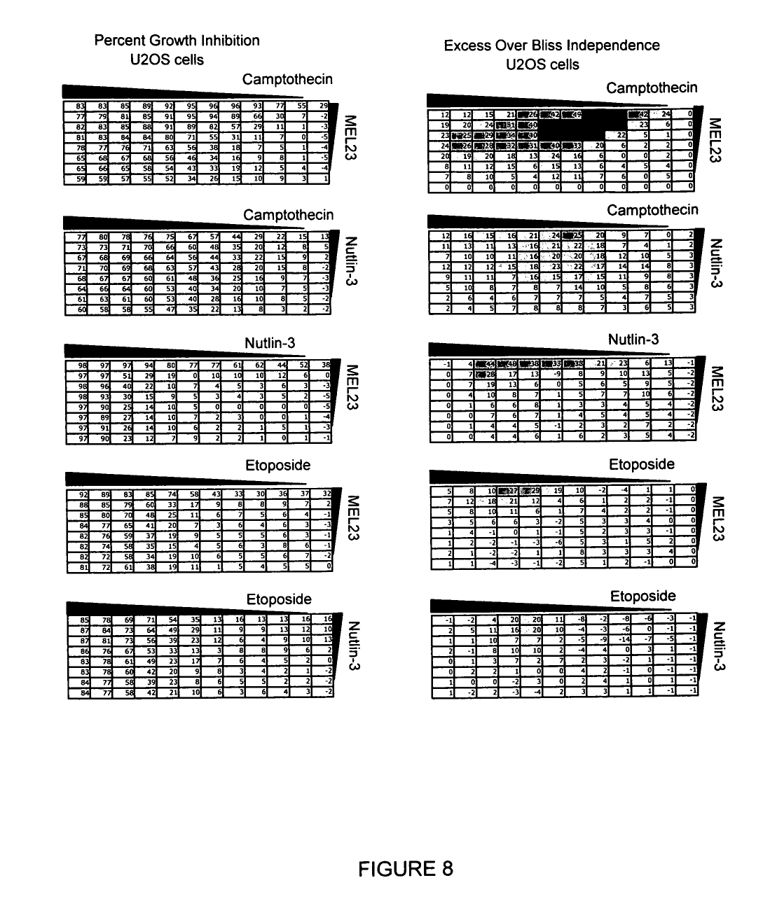

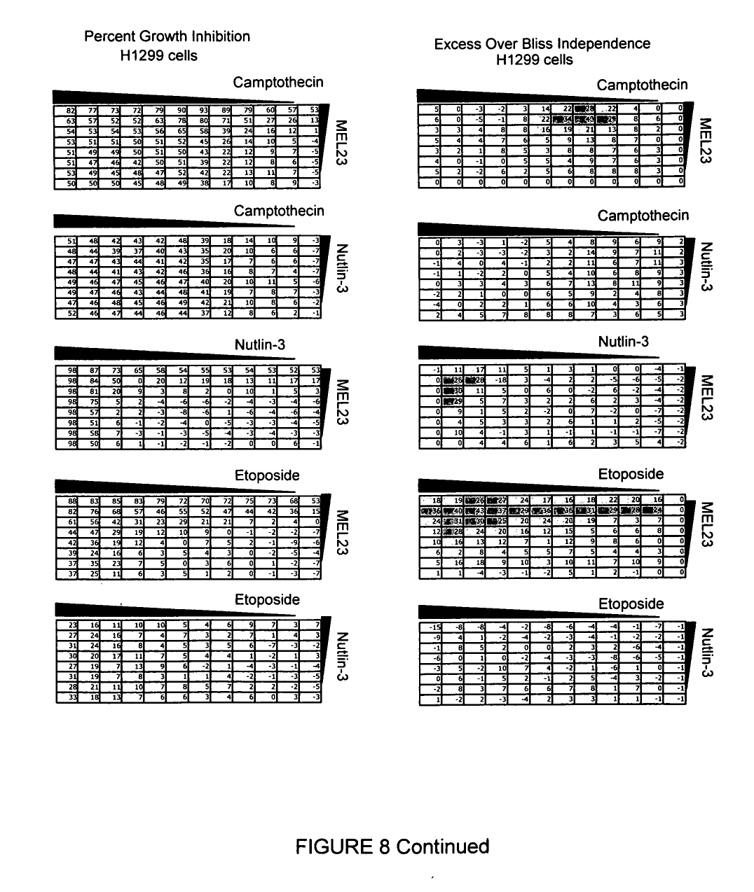

Because treatment with MEL compounds only led to a marginal increase in p53 target gene activation, the ability of MEL23 to cooperate with DNA-damaging agents to further activate p53 was tested. U2OS (p53 wild-type) and H1299 (p53-null) cells were treated with increasing amounts of MEL23 and either camptothecin or etoposide in combination for 48 hours, and then cell viability was measured. The combination of MEL23 with the DNA-damaging agents had a synergistic effect on viability (FIGS. 5B and 8). The results are presented as a decrease in cell viability over that predicted by the Bliss independence model. Bliss independence was determined by the formula C=A+B−A*B, where A is the treatment with one compound alone and B is treatment with the second compound alone (77). The excess over Bliss independence was determined by subtracting the predicted Bliss effect from the experimentally determined effect, yielding the synergistic effect of the combined treatment.

Because MEL23 can inhibit p53 ubiquitination and Nutlin-3 can prevent Mdm2 from inhibiting p53 transcriptional activation, the ability of MEL23 and Nutlin-3 to cooperatively decrease cell viability was tested. Synergy between MEL23 and Nutlin-3 occurred, but to a lesser extent than the combination of MEL23 and DNA-damaging agents (FIGS. 5B and 8).

Interestingly, MEL23 synergized with DNA damaging agents in p53-null H1299 cells. Nutlin-3, on the other hand had synergy with camptothecin and etoposide only in the p53-wild-type cell line. This may be due to the fact that the MEL compounds are inhibiting additional functions of Mdm2 that are p53-independent, but still potentially oncogenic. These synergistic effects of MEL23 with low concentrations of DNA damaging agents that do not cause deleterious effects on their own may increase the therapeutic utility of the MEL compounds.

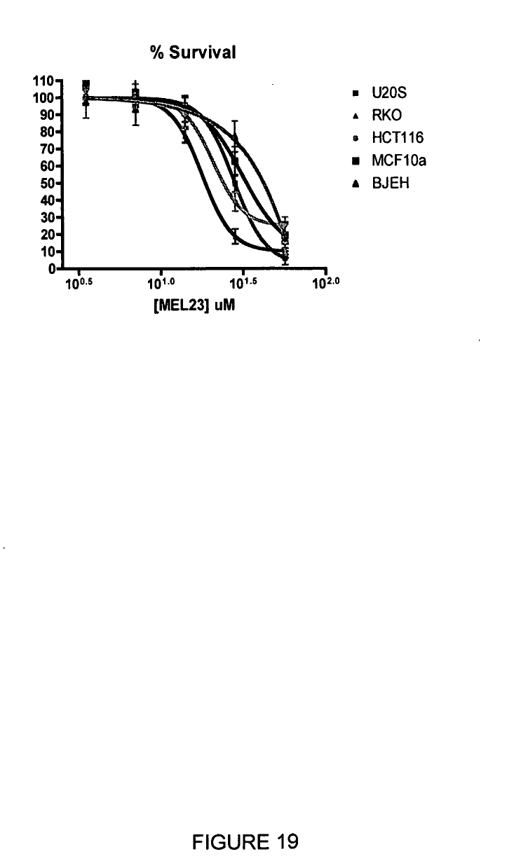

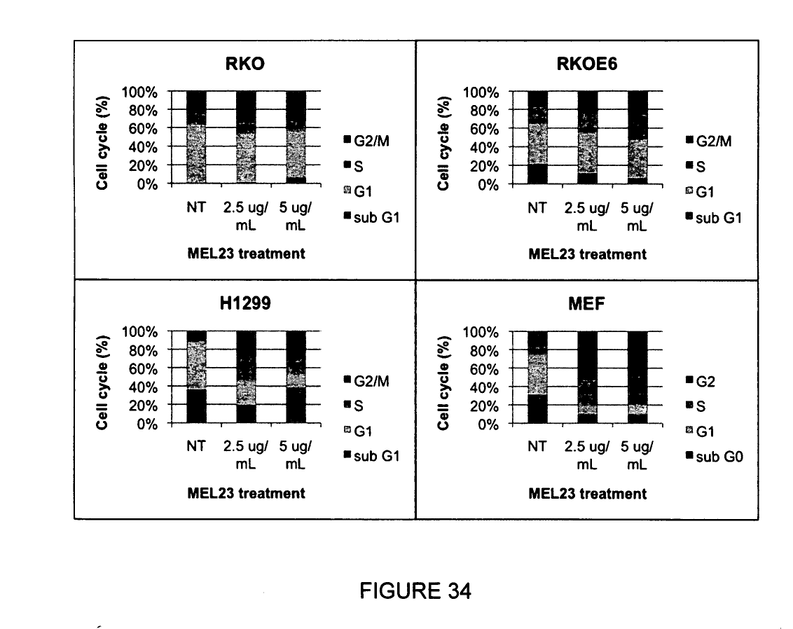

Thus, even though high concentrations were necessary to see inhibition of ubiquitination in vitro, the MEL compounds were able to induce p53 and Mdm2 accumulation in cells at much lower concentrations. It is possible that the enzymatic activity of the Mdm2-MdmX ligase is difficult to inhibit in vitro; even Nutlin compounds do not inhibit Mdm2 ubiquitination of p53 in vitro (78). Perhaps E3 ligase activity is only partially inhibited in cells; however, this might be enough to cause p53 accumulation, consistent with mouse data showing that just a 20-30% reduction in Mdm2 can lead to p53 activation (11). It is tempting to speculate that compounds which completely inhibited Mdm2 or Mdm2-MdmX E3 ligase activity would activate p53 in both cancer and normal cells to such a large extent that they would not be tolerated and would, therefore, not be useful as therapeutics. The MEL compounds, on the other hand, had a small but consistent differential between tumor derived and non-transformed cell lines, decreasing the survival of the tumor derived cells to a greater extent (FIG. 19). Cell cycle analysis revealed that the MEL24 treatment promotes G2 cell cycle arrest in H1299, U2OS, MEF, RKO and RK0-E6 cells, consistent with p53 activation in these cells (FIG. 34).

Because Mdm2 can inhibit p53 through two independent mechanisms—E3 ligase-mediated degradation and binding—it is formally possible that inhibitors of Mdm2 E3 ligase activity would not be sufficient to activate p53. In this model, Mdm2 E3 ligase inhibitors would increase both p53 and Mdm2 levels, but would not prevent Mdm2 from binding p53 and thereby inhibiting its activity. From the qRT-PCR data (FIGS. 10 and 16) and from co-immuniprecipitation data (FIG. 33), however, it is likely that Mdm2 and p53 are still interacting and Mdm2 is inhibiting p53 activity to some extent. However, increased p53 activity was observed compared to proteasome inhibition alone, which also increases Mdm2 and p53 levels. In RKO cells, such increase in p53 activity appears to be sufficient for p53-dependent cell death, albeit at higher concentrations of MEL23 than are needed for stabilization. Indeed, HLI compounds, Mdm2 E3 ligase inhibitors, also induce p53-dependent cell death, suggesting that complete disruption of Mdm2-p53 interaction may not be necessary for activation of p53 (15). Additionally, while MEL compounds do not activate p53 post-translational modifications, some cancer cells may already have such modifications in place as a consequence of oncogenic stress. Nevertheless, it is predicted that the combination of the MEL compounds and DNA damaging agents would enhance their therapeutic potential. This could also allow for use of DNA-damaging agents at concentrations that do not cause the deleterious side effects normally seen with cancer treatment.

MEL23 also cooperated with DNA-damaging agents in p53-null cells to a small, yet reproducible, extent. Mdm2 has been shown to have p53-independent oncogenic effects; for example, (a) overexpression of Mdm2 in mice causes tumors independent of p53 status (12), (b) splice variants of Mdm2 that cannot bind to p53 have been shown to be oncogenic (61), and (c) Mdm2 destabilization of Rb (62, 63) and p21 (64) may contribute to tumor growth. Therefore, the MEL compounds and other specific Mdm2 E3 ligase inhibitors may be beneficial in p53-null or p53-mutant tumors.

### Example 7

### Generation of MEL23/24 Analogue

### Compounds 12, 14, and 19

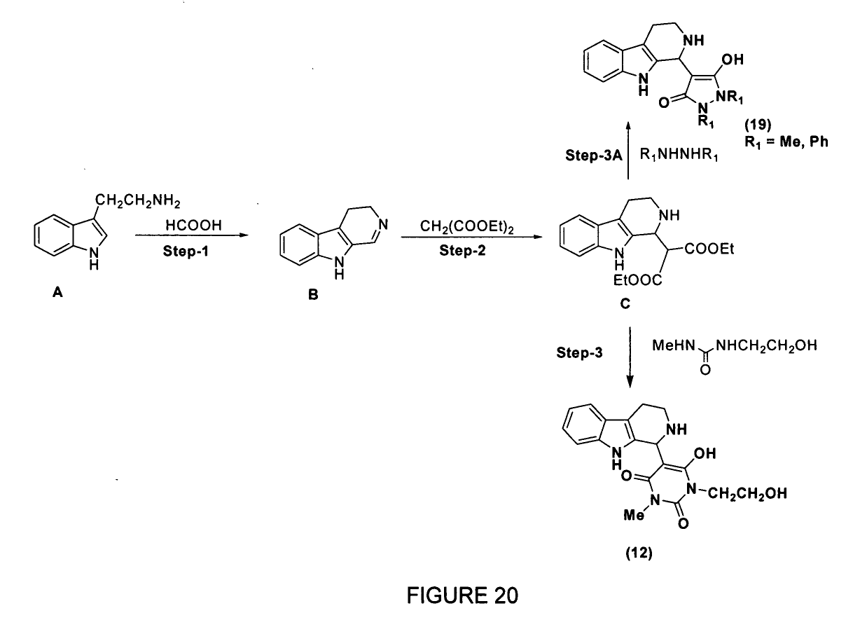

With reference to FIG. 20, compounds 12 and 19 may be synthesized as indicated.

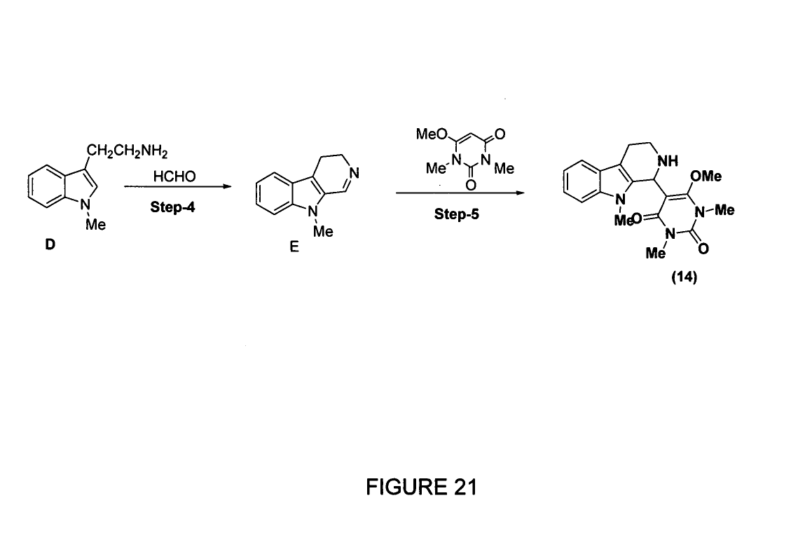

With reference to FIG. 21, compound 14 may be synthesized as indicated.

### Example 8

### Generation of MEL23/24 Analogue

### Compound 16

### Imine Formation

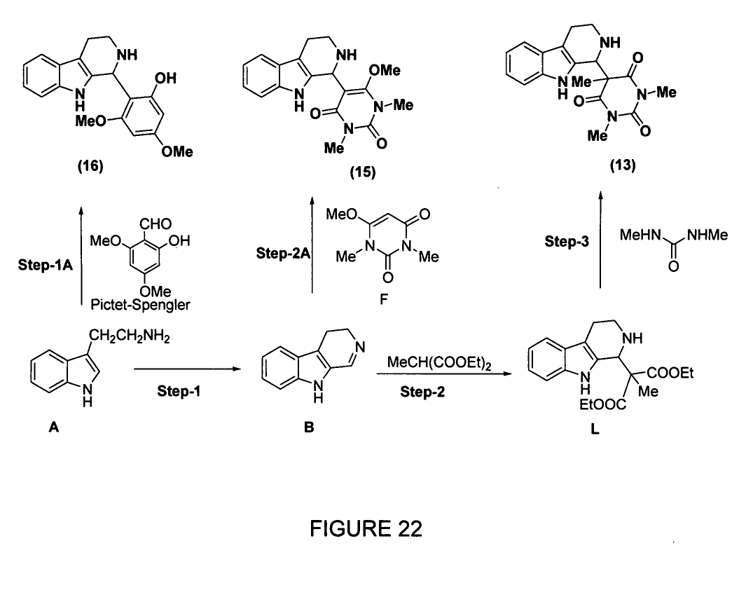

The synthetic scheme for making compound 16 is shown in FIG. 22. To the stirred solution of tryptamine, compound A (1.0 equivalent) in methanol was added 4,6-dimethoxy salicylaldehyde (Cat. No. 138797, Aldrich) (1.0 equivalent). The reaction mixture was refluxed at 65° C. for 30 minutes. The solvent was evaporated, and the residue was diluted with water and extracted with ethyl acetate. The crude product was purified by trituration with ether to give 150 mg of intermediate imine in 70% yield with 98.8% HPLC purity. The desired imine product was confirmed by LCMS and ¹H NMR analysis. As much as 300 mg of intermediate imine in 73% yield with 99% HPLC purity was made. The desired imine product was confirmed by LCMS and ¹H NMR analysis.

### Cyclization

The mixture of imine (150 mg, 1 equivalent) and POCl₃ (0.437 ml, 7 equivalent) was heated at 110° C. for 10 minutes. The reaction mixture was added to the ice cold solution of saturated NaHCO₃ and extracted with ethyl acetate. The crude product was purified by flash column chromatography to give 50 mg of compound 16 in 16% yield with 97% HPLC purity. Although the M+ is not observed by liquid chromatography-mass spectrometry (LCMS) analysis, the ¹H NMR has features of the desired compound 16.

The LCMS analysis did not show the desired molecular ion (M+) peak (MW=324.37, Observed M⁺=405). Similar results were observed when the LCMS analysis carried out under basic medium too. The variable temperature ¹H NMR study in DMSO-d₆ at 50° C. and 70° C. was carried out and has shown no any changes to the original ¹H NMR spectrum. The ¹H NMR has features of the desired compound 16.

### Example 9

### Generation of MEL23/24 Analogue

### Compound 15

The synthetic scheme for making compound 15 is shown in FIG. 22. With reference to FIG. 22, step-1 and step-2A are set forth below.

### Step-1

The mixture of tryptamine, compound A (1.0 equivalent) and formic acid −96% (5.99 ml) (Cat. No. 695076, Aldrich, Banglore, India) was heated at 140° C. for 35 minutes in the microwave to give 300 mg of uncyclised imine in 51% crude yield with 32% purity by LCMS analysis. The crude as such was used for the next reaction.

Then, the mixture of imine (150 mg, 1 equivalent) and POCl₃ (0.437 ml, 7 equivalent) was heated at 110° C. for 10 minutes. The reaction mixture was added to the ice cold solution of saturated NaHCO₃ and extracted with ethyl acetate. 280 mg of crude cyclized imine was obtained. The desired product was observed by LCMS analysis. The crude purification by preparative HPLC is completed to give 35 mg of compound B in 14% yield with 96% HPLC purity. This was confirmed by LCMS and ¹H NMR analysis.

### Step-2A

Step-2A will be carried out as set forth in the synthetic scheme.

### Example 10

### Generation of MEL23/24 Analogue

### Compound 13

The synthetic scheme for making compound 13 is shown in FIG. 22. With reference to FIG. 22, step-1, step-2, and step-3 are set forth below.

### Step-1

The mixture of tryptamine, compound A (1.0 equivalent) and 96% formic acid (5.99 ml) was heated at 140° C. for 35 minutes in the microwave to give 300 mg of uncyclised imine in 51% crude yield with 32% purity by LCMS analysis. The crude imine as such used for the next reaction.

Then, the mixture of imine (150 mg, 1 equivalent) and POCl₃ (0.437 ml, 7 equivalent) was heated at 110° C. for 10 minutes. The reaction mixture was added to the ice cold solution of saturated NaHCO₃ and extracted with ethyl acetate. 280 mg of crude cyclized imine was obtained. The desired product was observed by LCMS analysis. The crude purification by preparative HPLC is completed to give 35 mg of compound B in 14% yield with 96% HPLC purity. This was confirmed by LCMS and ¹H NMR analysis.

### Step-2 and Step-3

Step-2 and step-3 will be carried out as set forth in the synthetic scheme.

### Example 11

### Generation of MEL23/24 Analogue

### Compound 19

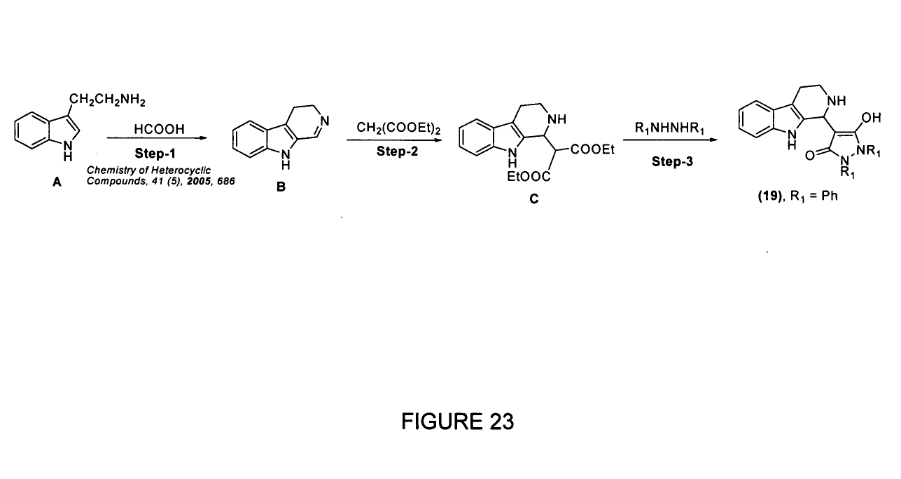

The synthetic scheme for making compound 19 is shown in FIG. 23. With reference to FIG. 23, step-1, step-2, and step-3 are set forth below.

### Step-1

Step-1 is based on reaction steps disclosed by Semenov et al. (44). A mixture of tryptamine, compound A (Cat. No. 19374-7, Aldrich, Banglore, India) (1.0 equivalent) and ethyl formate (Cat no. 74616, Thomas Baker, Mumbai, India) (10.0 equivalent) was heated at 55° C.-60° C. for 12 hours to give up to 5.1 grams of uncyclised imine in 87% yield with 95% purity by LCMS analysis. This was confirmed ¹H NMR analysis.

Then, the mixture of imine (150 mg, 1 equivalent) and POCl₃ (0.437 ml, 7 equivalent) was heated at 110° C. for 10 minutes. The reaction mixture was added to the ice cold solution of saturated NaHCO₃ and extracted with ethyl acetate. 5.1 grams of uncyclised imine gave 4 g of cyclized imine, compound B, in 86% yield with 97% HPLC purity. This is confirmed by LCMS and ¹H NMR analysis.

### Step-2

To the stirred solution of compound B, diethyl malonate (Cat. No. D9775-4, Aldrich) (1.5 equivalents) in tetrahydrofuran (THF) (3 ml) was added triethyl amine (1.5 equivalents) and the reaction mixture was stirred at room temperature for 12 hours. The reaction mixture was diluted with 1N HCl and extracted with ethyl acetate. The desired product, compound C, was observed by LCMS analysis (47%). This step may be scaled to give 50 mg of compound C in 5% yield with 73% HPLC purity, as confirmed by LCMS and ¹H NMR analysis.

### Step 3

Step-3 will be carried out as set forth in the synthetic scheme, with, e.g., diphenyl hydrazine (Cat. No. 12672, Aldrich) and diethyl malonate.

### Example 12

### Alternate Synthetic Schemes for MEL23/24 Analogue, Compound 15

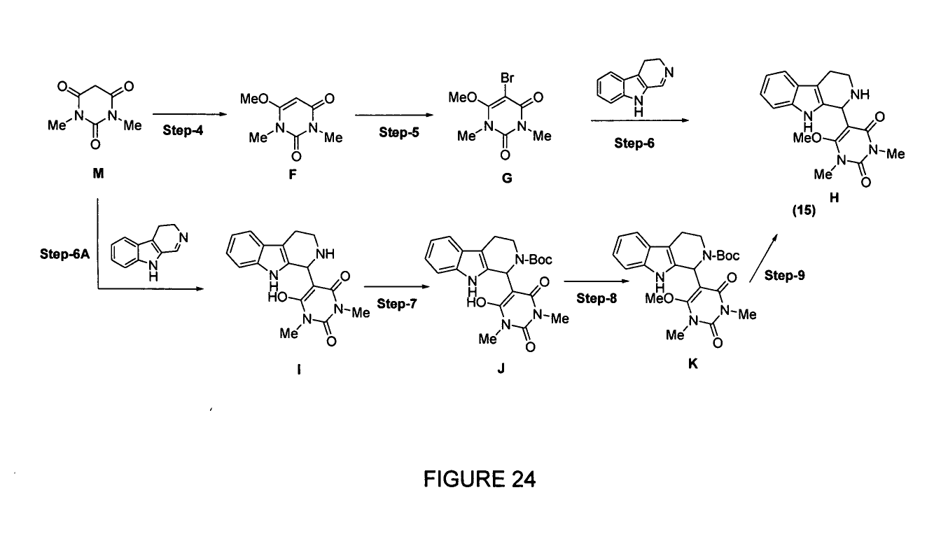

Two alternate synthetic schemes for making compound 15 are shown in FIG. 24. With reference to FIG. 24, step-4, step-5, step-6, step-6A, step-7, step-8, and step-9 are set forth below.

### Step-4

Step 4 was designed based on the disclosure by Szczepankiewicz et al. (45) To the stirred solution of 1,3-dimethyl barbituric acid (compound M) in MeOH:ethyl acetate (1:5, 20 mL) was added trimethylsilyl-diazomethane (Cat. No. 362832, Aldrich) (7.0 equivalents) at 0° C. Then, the reaction mixture was stirred at room temperature for 15 minutes. Solvent was evaporated and the reaction mixture was diluted with water and extracted with ethyl acetate. Purification was done by trituration with ether:hexane to give 600 mg of compound F in 55% yield with 98% HPLC purity. This is confirmed by LCMS and ¹H NMR analysis.

### Step-5

To the stirred solution of compound F in CCl₄, α,α′-Azoisobutyronitrile (Cas no. 78-67-1) used in the reaction in a catalytic amount (e.g. 0.1 equivalent) was added NBS (1.1 equivalents) and heated at 45° C. for 1 hour. CCl₄ was evaporated. Then, the residue was diluted with water and extracted with ethyl acetate to give 200 mg of crude compound G. The desired M+ is observed by LCMS analysis. The crude product (170 mg) was purified by flash column chromatography to give 50 mg of compound G in 17% yield with 70% HPLC purity. This is confirmed by LCMS and ¹H NMR analysis.

### Step-6

Step-6 will be carried out as set forth in the synthetic scheme to provide compound 15.

### Step-6A

The following procedure for step-6A is based on the disclosure of Semenov et al. (44). A mixture of compound B, 1,3-dimethyl barbituric acid (compound M, Cat. No. 31800-0, Aldrich) (1.0 equivalent) in CCl₄ (5 mL) was set at 60° C. for 15 minutes, followed by stirring at room temperature for 12 hours. The desired compound I was observed by LCMS analysis but in very low percentage (14%).

Alternatively, step-6A was carried out as follows (46). To a stirred solution of compound B in hot ethanol (3 mL), was added a hot solution of 1,3-dimethyl barbituric acid (compound M) (1.0 equiv) in ethanol (2 mL) at 40° C. for 15 minutes, followed by stirring at room temperature for 12 hours. A precipitate was formed, which was filtered through a Buckner funnel to give up to 30 mg of compound I in 16% yield with 77% HPLC purity. This was confirmed by LCMS and ¹H NMR analysis. This procedure was repeated to give 200 mg of compound I in 21% yield with 73% HPLC purity. This was confirmed by LCMS and ¹H NMR analysis.

Yet another alternative is as follows: To the stirred solution of compound B, Boron trifluoride diethyl etherate (Cat. No. 15720, Fluka, Banglore, India) (in catalytic amount, e.g., 0.1 equivalent) in hot ethanol (3 mL), was added a solution of 1,3-dimethyl barbituric acid (compound M) (1.0 equivalent), potassium carbonate (1.0 equivalent) in hot ethanol (2 ml), which was followed by stirring at room temperature for 2 hours. A precipitate was formed, which was filtered through a Buckner funnel to give 130 mg of orange-colored crude compound I in 39% yield. The desired compound I is confirmed by LCMS analysis and NMR.

An additional method is as follows: To the stirred solution of compound B, trifluoro acetic anhydride (cas no. 76-05-1, S.d. Fine-Chem. Ltd., Pune, India) (1.0 equivalent) in hot ethanol (3 ml), was added a solution of 1,3-dimethyl barbituric acid (compound M) (1.0 equivalent), potassium carbonate (1.0 equivalent) in hot ethanol (2 ml), which was followed by stirring at room temperature for 2 hours. A precipitate was formed, which was filtered through a Buckner funnel to give a crude product, which was purified by trituration with ethanol to give 50 mg of orange-colored crude compound I in 26% yield. The desired compound I is confirmed by LCMS analysis.

### Step-7

To the stirred solution of compound I, DMAP (1.0 equivalent) in dry DCM, was added Boc anhydride (1.5 equivalents) at 0° C. and stirring continued at room temperature for 1 hour. The reaction mixture was diluted with water and extracted in ethyl acetate to give crude compound, which was purified by flash column chromatography to give 170 mg of compound J in 70% yield with 93% HPLC purity. This was confirmed by LCMS analysis and NMR.

The procedure above was repeated. The purification was done by flash column chromatography to give 185 mg of compound J in 81% yield with 87% HPLC purity. This is confirmed by LCMS and ¹H NMR analysis.

### Step-8

To the stirred solution of compound J in ethyl acetate:methanol solution (3:2, 0.64 ml) was added trimethylsilyl diazomethane (Cat. No. 362832, Aldrich) at 0° C., and the mixture was stirred for 30 minutes. A crude purification by prep TLC was completed to give 6 mg of compound K, which shows the M⁺=340−deprotected compound K, i.e., Compound 19 by LCMS analysis. ¹H NMR did not show the convincing results.

The procedure set forth above was repeated. The crude purification by prep TLC was completed to give 5 mg of compound K which shows the M⁺=340−deprotected K, i.e., Compound 19 by LCMS analysis.

The procedure set forth above was repeated again. The desired compound K was observed by LCMS analysis (52%). The crude compound (210 mg) was used for the next reaction.

### Step-9

A mixture of crude compound K in 1,4-dioxane. HCl (3 ml) was stirred at room temperature for 2 hours. The solvent was evaporated and crude solid was diluted with 1 N HCl and extracted in ethyl acetate. The crude may be purified by prep HPLC.

### Example 13

### Alternate Synthetic Scheme for MEL23/24 Analogue, Compound 14

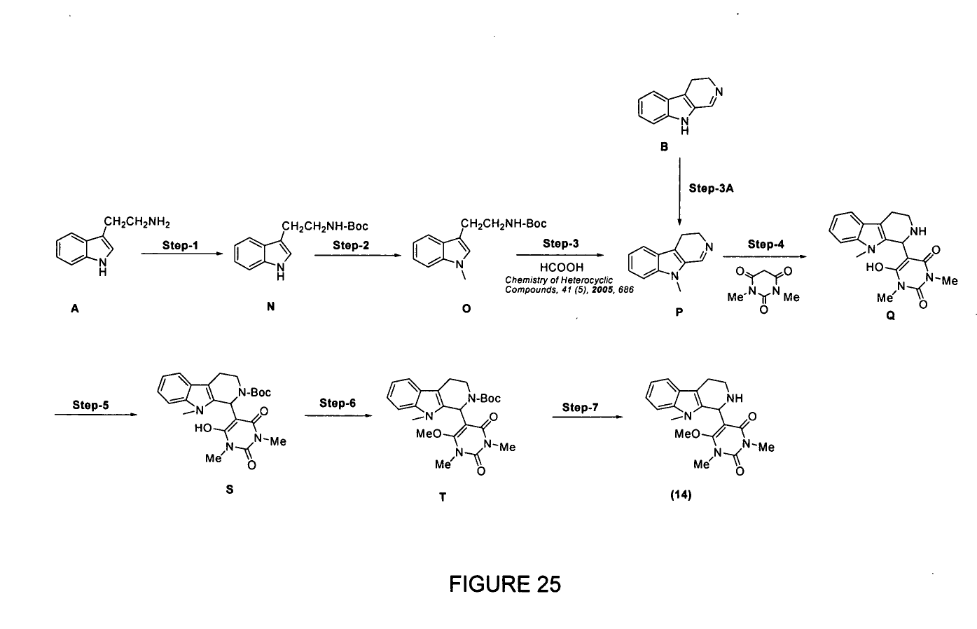

An alternate synthetic scheme for making compound 14 is shown in FIG. 25. With reference to FIG. 25, steps 1-7 are set forth below.

### Step-1

To the stirred solution of A in dry THF was added NaHCO₃ (1.0 equivalent) and Boc anhydride (1.2 equivalents) at 0° C. and stirring continued at room temperature for 1 hour. The reaction mixture was diluted with water and extracted in ethyl acetate. Solvent was evaporated to give 100 mg of crude N. The desired compound is not observed by LCMS but confirmed by ¹H NMR analysis. This reaction was also scaled up to give 330 mg of compound N in 41% yield with 98% HPLC purity. The desired compound is not observed by LCMS but confirmed by ¹H NMR analysis.

### Step-2

Step-2 may be carried out as set forth in the synthetic scheme.

### Step-3A

To a stirred solution of n-Butyl Lithium (Cat. No. 18617-1, Aldrich) (1.5 equivalent) in dry THF, was added drop wise a solution of compound B in dry THF at −10° C. and stirred the reaction mixture at −10° C. under nitrogen for 1 hour. Then methyl iodide (4.9 equiv.) was added, and the mixture was stirred at room temperature for 0.5 hours. The reaction mixture was diluted with 1N HCl and extracted in ethyl acetate. The desired compound P was observed by LCMS analysis (20%) and for crude purification this batch is mixed with the next batch (79).

### Steps 3-7

Steps 3-7 will be carried out as set forth in the synthetic scheme.

### Example 14

### Alternate Synthetic Scheme for MEL23/24 Analogue, Compound 19

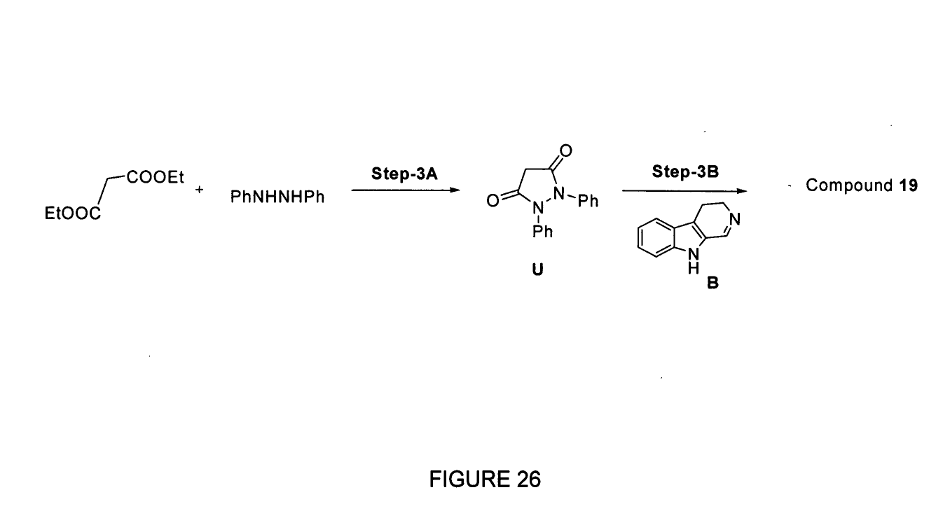

An alternate synthetic scheme for making compound 19 is shown in FIG. 26. With reference to FIG. 26, step-3A and step-3B are set forth below.

### Step-3A

To the stirred solution of 1,2 diphenyl hydrazine (Cat. No. 126721, Aldrich), freshly prepared NaOMe (10 equiv.) in n-butanol was added diethyl malonate (Cat. No. D9775-4, Aldrich) (1.5 equivalent) and heated at 100° C. for 12 hours. This reaction was completed and the desired compound U was observed by LCMS analysis (62%). The degradation of the product is observed during the flash silica column. (80).

This procedure was scaled to 1.0 grams, with desired compound U observed by LCMS analysis (30%). A part of the crude product (300 mg, 30% by LCMS) may be used for the next step. Degradation of the product is observed during the purification by HPLC.

### Step-3B

Step-3B may be carried out as set forth in FIG. 26.

### Example 15

### Testing for Direct Binding of the MEL23/24 Scaffold to Mdm2 and Generation of MEL23/24 Analogues

A radiolabeled analog of MEL23/24 may be made, and a filter-binding assay may be used to measure direct binding to Mdm2. A similar protocol has been used to measure binding of ATP analogs to the RING domain of Mdm2 (34). Full-length Mdm2 protein, as well as many deletion constructs and fragments of Mdm2, have been purified (34-37, 42-43).

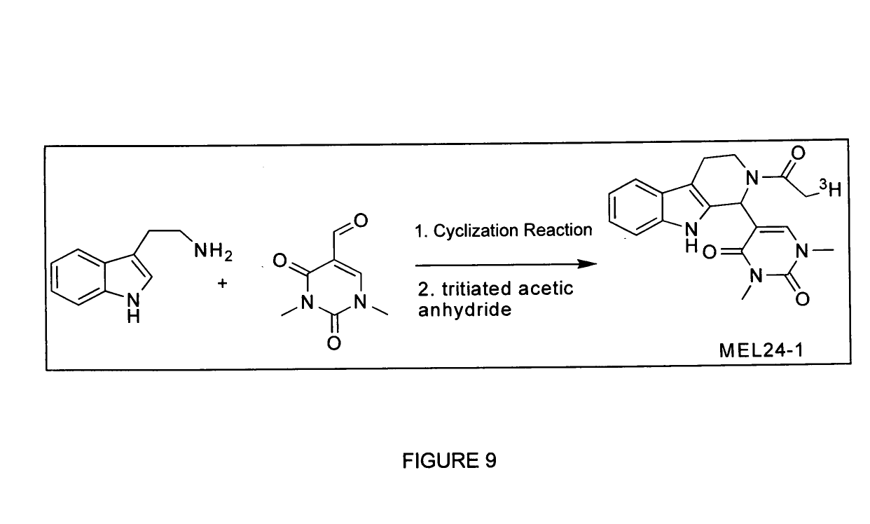

To synthesize a radiolabeled analog of MEL24, tritiated acetic anhydride will be used to install a tritiated acetyl group on the beta-cerboline secondary amine of MEL24 (FIG. 9, MEL24-1). One concern with performing this reaction on MEL24 itself is that acylation of that nitrogen will render the adjacent carbon-carbon labile, probably resulting in opening of the beta-carboline through a retro-Michael-type mechanism. To avoid this scenario, an analog of MEL24 lacking the hydroxyl group needed for this retro-Michael reaction (MEL24-1) will instead be synthesized. This analog will be synthesized via, e.g., a Pictet-Spengler cyclization of tryptamine with a suitable aldehyde or another suitable cyclization reaction, then acetylated with tritiated acetic anhydride (FIG. 9). The cold analog of this compound will also be made in order to determine that it is active. If it is not active, other analogs of MEL23/24 will be synthesized using the same Pictet-Spengler cyclization chemistry, varying the site of addition of the radiolabel.

This synthetic route may be generalized to provide for racemic MEL23/24 analogs. The enantiomers will be purified by HPLC using a chiral column. However, asymmetric variants of the Pictet-Spengler cyclizations obviating the need for chiral separation may also be employed.

### Example 16

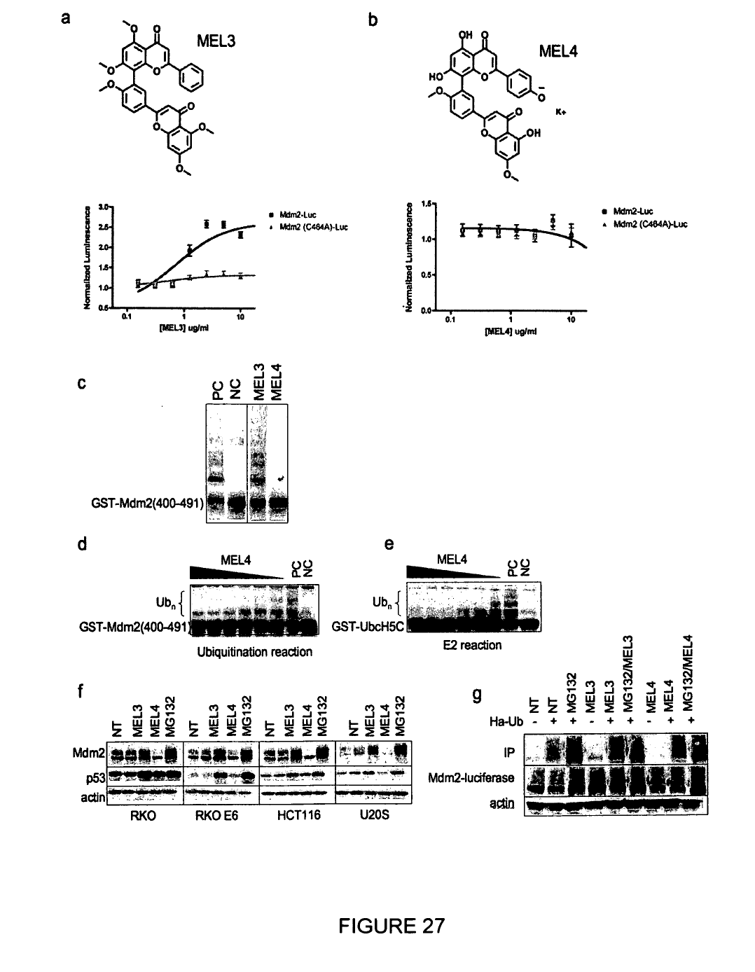

Referring now to FIGS. 27a and b, the structures of MEL3 and MEL4 and their activities in the cell-based auto-ubiquitination assay are shown. Briefly, cells were plated in 384-well format and treated with MEL3 or MEL4 for 2 hours at the indicated concentrations. A luminescence buffer was added and the relative luminescence levels determined on a Victor3 Plate Reader. Each graph represents the median of three replicates normalized to a no treatment control; one standard deviation is shown. The EC₅₀ value of MEL3 was determined to be 0.7 μg/ml (1.2 μM). The graphs and EC₅₀ values were determined using Graphpad Prism.

Referring now to FIGS. 27c and d, in vitro ubiquitination reactions with dilutions of MEL3 and MEL4 are shown. Briefly, 25 ng of E1, 25 ng of Ubch5C, 1 μg of ubiquitin, and 500 ng of GST-Mdm2(400-491) were incubated with or without the indicated compound for 1 hour at 37° C. in ubiquitination buffer. Samples were analyzed by Western blotting with anti-GST. PC indicates positive control and NC indicates negative controls without UbCH5C protein. In FIG. 27c, compounds were tested at 120 μg/ml, and in FIG. 27(d) the MEL4 was tested in a 2-fold dilution series, 100, 50, 25, 12.5, 6.25, and 3.125 μg/ml. Brackets indicate slower migrating species of the proteins are the ubiquitin-conjugated forms.

Referring now to FIG. 27e, an in vitro E2 ubiquitination reaction with dilutions of MEL4 is shown. Briefly, 25 ng of E1, 500 ng of GST-Ubch5C, and 1 μg of ubiquitin were incubated with and without the indicated compound for 1 hour at 37° C. in ubiquitination buffer. Samples were analyzed by Western blotting with anti-GST. PC indicates positive control and NC indicates negative controls without E1 protein. MEL4 was tested in a 2-fold dilution series, 100, 50, 25, 12.5, 6.25, and 3.125 μg/ml. Brackets indicate slower migrating species of the proteins are the ubiquitin-conjugated forms.

Referring now to FIG. 27f, endogenous Mdm2 and p53 protein levels are increased with MEL3 but not MEL4 treatment as shown. Briefly, U2OS, RKO, and HCT116 cells (p53 wild-type) and RKO E6 cells were incubated with 5 μg/ml of MEL3 or MEL4, or 10 μM of MG132 for 6 hours. Cells were lysed and analyzed by Western blotting using anti-Mdm2, anti-p53, and anti-actin antibodies. The lower band seen with the Mdm2 antibody is a non-specific band. NT indicates no treatment control.

Referring now to FIG. 27g, MEL3 and MEL4 did not inhibit Mdm2 auto-ubiquitination in cells. Briefly, Mdm2-luciferase cells were transfected with HA-ubiquitin. 36 hours later cells were treated with 5 μg/ml of MEL3 or MEL4 and co-treated with 10 μM MG132 or a DMSO control. 2 hours later, cells were lysed and immunoprecipitated for 3 hours with an anti-HA affinity matrix. Input lysates and immunoprecipitated samples were analyzed by Western blotting using anti-Mdm2 antibody.

### Identification of Primary Hits: MEL3 and MEL4

MEL3 and MEL4 are structural analogs identified from the Microsource library (FIGS. 27a and 27b). MEL3 showed consistent activity in the cell-based assay, increasing the luminescence of the Mdm2-luciferase cell line without significantly affecting the luminescence of the control Mdm2(C464A)-luciferase cell line (FIG. 27a). After re-testing in the cell-based assay, MEL4 did not show any activity (FIG. 27b). The EC₅₀ value of MEL3 was determined to be 0.7 μg/ml (1.2W).

In the in vitro ubiquitination reaction, only MEL4, but not MEL3, had activity (FIGS. 27c and 27d). It seems likely that MEL4 inhibits the E1 or UbCH5C protein, as it is active in both the Mdm2 ubiquitination and E2 ubiquitination reactions at similar concentrations (FIGS. 27d and 27e). It was hypothesized that only MEL3 can get into cells where it is then modified to the active form of the compound MEL4, which would explain the activity of MEL3 in cells and MEL4 in vitro. In view of the foregoing, these analogs were studied further, as disclosed in more detail below.

An Mdm2 E3 ligase inhibitor should prevent degradation of Mdm2 substrates, including Mdm2 and p53. Further testing showed that MEL3, but not MEL4, is able to increase Mdm2 and p53 levels in three wild-type p53 expressing cell lines, RKO, HCT116, and U2OS (FIG. 27f). This is consistent with only MEL3 being active in cells.

Many compounds can lead to increases in both Mdm2 and p53 levels by inducing a DNA damage response. The RKO and RKO E6 isogenic cell lines were used to test if MEL3 was inducing a DNA damage response and to test if the compound was specific for Mdm2. RKO and RK0-E6 cells have wild-type p53 and Mdm2, but RK0-E6 cells express the HPV-E6 protein. The HPV-E6 protein forms an active E3 ligase in association with E6AP, a HECT domain E3 ligase, and targets p53 for degradation (69). As a result, p53 levels are significantly reduced in RK0-E6 cells compared to the RKO cells (FIG. 27f, NT lanes). A compound that does not induce a DNA damage response or that specifically inhibits Mdm2 E3 ligase activity should increase p53 levels in RKO cells, but not in RKO-E6 cells, as the compound should not affect E6AP activity. It was found that MEL3 increases p53 levels in both RKO and RKO E6 cell lines (FIG. 27f) indicating that the compound is either stimulating a DNA response or is inhibiting the E6AP HECT domain E3 ligase. Inhibition of E6AP is consistent with the in vitro findings that MEL3 can inhibit the E1 or E2, UbCH5C, protein.

To determine whether MEL3 and MEL4 can inhibit Mdm2 ubiquitination in cells, Mdm2-ubiquitin conjugates were analyzed with a cell-based ubiquitination assay (FIG. 27g). The Mdm2-luciferase cell line was transfected with HA-ubiquitin and co-treated with 5 μg/ml MEL3 or MEL4 and 10 μM MG132 or a DMSO control. Cell lysates were immunoprecipitated with an anti-HA antibody and analyzed by western blotting. In cells treated with MEL3 or MEL4, similar amounts of Mdm2-ubiquitin conjugates were immunoprecipitated, indicating that the compounds do not inhibit ubiquitination of Mdm2 in this assay. It is possible that further optimization of this assay, such as pretreating the cells with MEL3 or MEL4 before MG132 or increasing the concentration of compound used, may result in some inhibition, but that has not been done to date.

It would be interesting to further analyze whether one of these analogs is the active form, MEL4, but the other is able to get across the cell membrane, MEL3. As E2 or E1 inhibitors may have both scientific and clinical benefits, future studies with these compounds, to determine their exact mechanism of action is warranted.

### Example 17

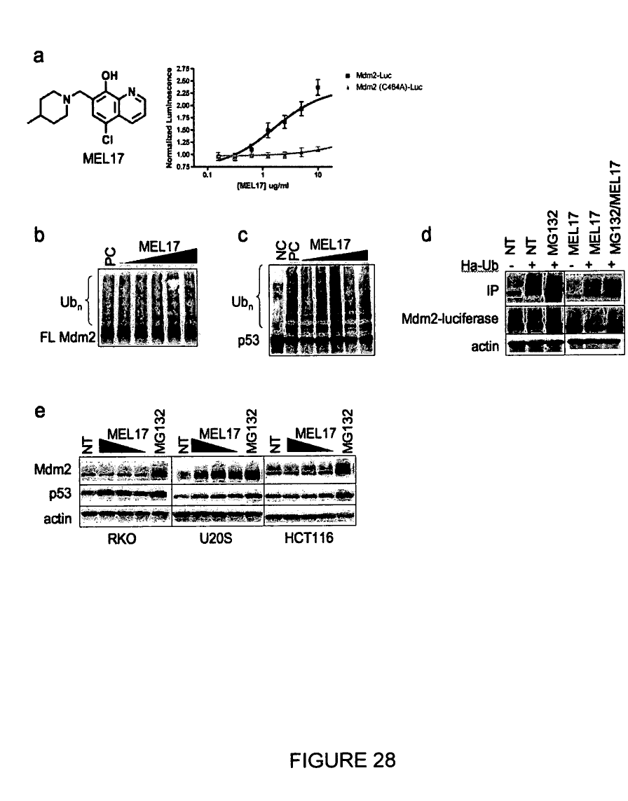

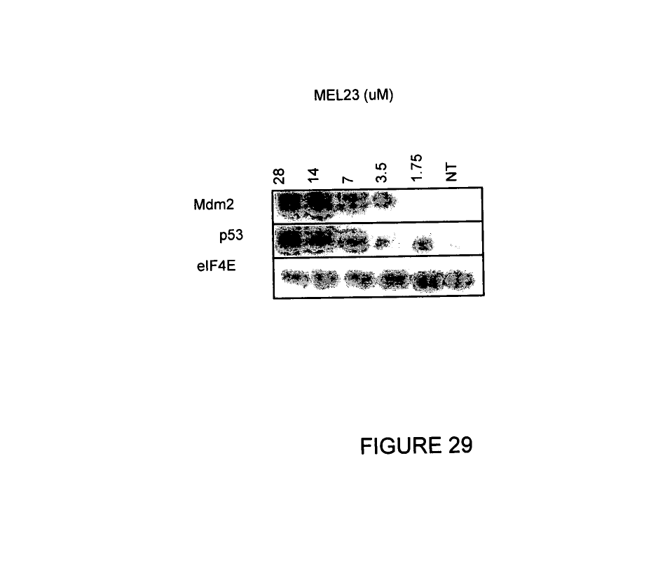

Referring now to FIG. 28a, the structure of MEL17 and its activity in the cell-based auto-ubiquitination assay are shown. Briefly, cells were plated in a 384-well format and treated with MEL17 for 2 hours at the indicated concentrations. A luminescence buffer was added and the relative luminescence levels determined on a Victor3 Plate Reader. Each graph represents the median of three replicates normalized to a no treatment control; one standard deviation is shown. The EC₅₀ value of MEL17 was determined to be 1.6 μg/ml (5.5 μM). The graphs and EC₅₀ values were determined using Graphpad Prism.

Referring now to FIG. 28b, as shown, Mdm2 in vitro ubiquitination is not inhibited by MEL17 treatment. Briefly, 50 ng of E1, 50 ng of Ubch5C, 1 μg of PK-ubiquitin, and 300 ng of full-length Flag-Mdm2 were incubated for 1 hour at 37° C. in ubiquitination buffer. Samples were analyzed by Western blotting with anti-Mdm2 antibodies. PC indicates positive control and NC indicates negative control lacking UbCH5C. Reactions were performed in the presence of 5, 10, 20, 50, and 100 μg/ml of MEL17 compound. Brackets indicate slower migrating species of the proteins are the ubiquitin-conjugated forms.

Referring now to FIG. 28c, as shown, p53 in vitro ubiquitination reactions are not inhibited by MEL17 treatment. Briefly, 50 ng of E1, 50 ng of Ubch5C, 1 μg of PK-ubiquitin, 300 ng of full-length Flag-Mdm2, and 50 ng of p53 were incubated for 1 hour at 37° C. in ubiquitination buffer. Samples were analyzed by Western blotting with anti-p53 antibodies. PC indicates positive control and NC indicates negative control lacking UbCH5C. Reactions were performed in the presence of 5, 10, 20, 50, and 100 μg/ml of MEL17 compound. Brackets indicate slower migrating species of the proteins are the ubiquitin-conjugated forms.

Referring now to FIG. 28d, as shown, MEL17 inhibits Mdm2 auto-ubiquitination in a cell-based assay. Briefly, Mdm2-luciferase cells were transfected with HA-ubiquitin. 36 hours later, cells were treated with 5 μg/ml of MEL17 and co-treated with 10 μM MG132 or a DMSO control. 2 hours later, cells were lysed and immunoprecipitated for 3 hours with an anti-HA affinity matrix. Input lysates and immunoprecipitated samples were analyzed by Western blotting using anti-Mdm2 antibody. NT indicates no treatment control.

Referring now to FIG. 28e, endogenous Mdm2 and p53 protein levels with MEL17 treatment are shown. Briefly, U2OS, RKO, and HCT116 cells (p53 wild-type) were incubated with 5 μg/ml of MEL17 or 10 μM of MG132 for 6 hours. Cells were lysed and analyzed by Western blotting using anti-Mdm2, anti-p53, and anti-actin antibodies. The lower band seen with the Mdm2 antibody is a non-specific band.

### Identification of Primary Hits: MEL 17

MEL17 was identified from the BBB library and showed consistent activity in the cell-based assay, increasing the luminescence of the Mdm2-luciferase cell line without significantly affecting the luminescence of the control Mdm2(C464A)-luciferase cell line (FIG. 28a). The EC₅₀ value of MEL17 was determined to be 1.6 μg/ml (5.5 μM).

MEL17 does not inhibit full-length Mdm2, RING Mdm2, or p53 ubiquitination when analyzed with purified proteins in vitro (FIGS. 28b and 28c). Full-length Mdm2 and p53 ubiquitination were tested in addition to RING Mdm2, because MEL17 appeared to inhibit Mdm2 ubiquitination in cells (FIG. 28d). To determine whether MEL17 inhibits Mdm2 ubiquitination in cells, the Mdm2-luciferase cell line was transfected with HA-ubiquitin and co-treated with 5 μg/ml MEL17 and 10 μM MG132 or a DMSO control. Cells lysates were immunoprecipitated with an anti-HA antibody and analyzed by Western blotting. In cells treated with MEL17 or MEL17 and MG132, significantly less Mdm2-ubiquitin conjugates were immunoprecipitated, indicating that the compound inhibits ubiquitination of Mdm2 in this assay (FIG. 28d). Although this assay was repeated multiple times, the results were variable. It is possible that further optimization of this assay, such as pre-treating the cells with MEL17 before MG132 treatment, may result in an increased and more robust inhibition, but that has not been done to date. One likely possibility for the discrepancy between the in vitro and cell-based ubiquitination assays is that a domain on Mdm2 other than the RING domain interacts with MEL17. In vitro, the RING domain alone is sufficient for auto-ubiquitination of Mdm2, so inhibitors of other domains may be undetectable.

An Mdm2 E3 ligase inhibitor should prevent the degradation of endogenous Mdm2 substrates, including Mdm2 and p53. Further testing showed that MEL17 increases Mdm2 and p53 levels in certain, but not all, wild-type p53 expressing cell lines tested (FIG. 28e). Treatment of 5 μg/ml MEL17 increased p53 levels in RK0 and U2OS but not HCT116 cells and increased Mdm2 in U2OS but not RKO or HCT116. As MEL17 did not increase Mdm2 and p53 in all three cell lines tested, it is likely not directly inhibiting Mdm2 E3 ligase activity.

MEL17 was also sent to the National Institutes of Health (NIH) for analysis in the NCl60 screen. This is a service provide by the NIH, that tests novel compounds for killing in 60 tumor cell lines. As MEL17 displayed a unique and interesting killing profile in these 60 tumor cell lines, it was selected for preliminary in vivo mouse studies. First, MEL17 was tested for toxicity and it was found that it was non-toxic at all concentrations tested up to 400 mg/kg/dose. It was then tested in a hollow-fiber assay. 12 tumor cell lines were tested in two concentrations and at two sites, subcutaneous implants and intraperitoneal implants. A 50% or greater reduction in percent net growth in the treated samples compared to the vehicle control was considered a positive result, and given a score of 2. Out of a possible score of 96 (12 cell lines×2 concentrations×2 sites×a score of 2), MEL17 got a score of 4. As it had a low score, MEL17 did not qualify for further testing by the NIH.

MEL17 did not increase p53 and Mdm2 in all the cell lines tested, and therefore, it has not been analyzed further. However, testing MEL17 in a broader range of p53 wild-type cell lines may give a clearer picture of MEL17's ability to increase endogenous Mdm2 and p53. From both cell-based and in vitro testing, it seems likely that Mdm2 is not the direct target of MEL17. However, despite this, it may be interesting to determine MEL17's mechanism of activation and how it can induce a unique tumor cell death profile whether or not this is directly dependent on the p53-Mdm2 pathway.

### Example 18

### Synthetic Scheme for MEL24 Analogue, Compound 20

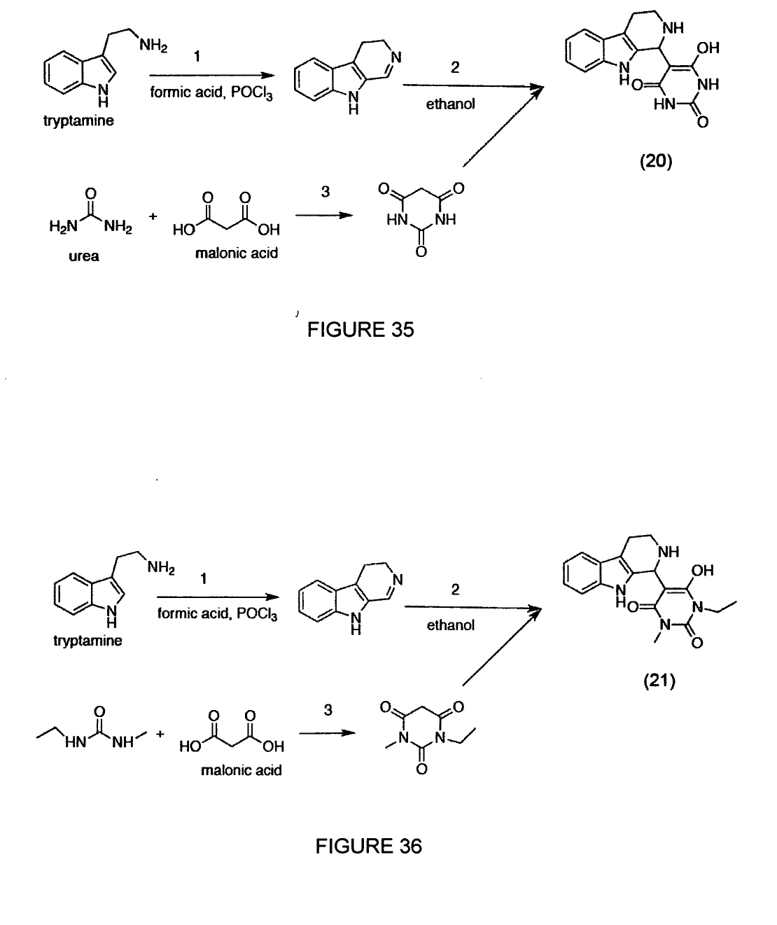

A representative synthetic scheme for making compound 20 is shown in FIG. 35. With reference to FIG. 35, step 1 of the reaction may be carried out as set forth in Example 10, Step-1 above. Steps 2 and 3 of the reaction may be carried out as set forth in FIG. 35.

### Example 19

### Synthetic Scheme for MEL24 Analogue, Compound 21

A representative synthetic scheme for making compound 21 is shown in FIG. 36. With reference to FIG. 36, step 1 of the reaction may be carried out as set forth in Example 10, Step-1 above. Steps 2 and 3 of the reaction may be carried out as set forth in FIG. 36.

### Example 20

### Synthetic Scheme for MEL24 Analogue, Compound 22

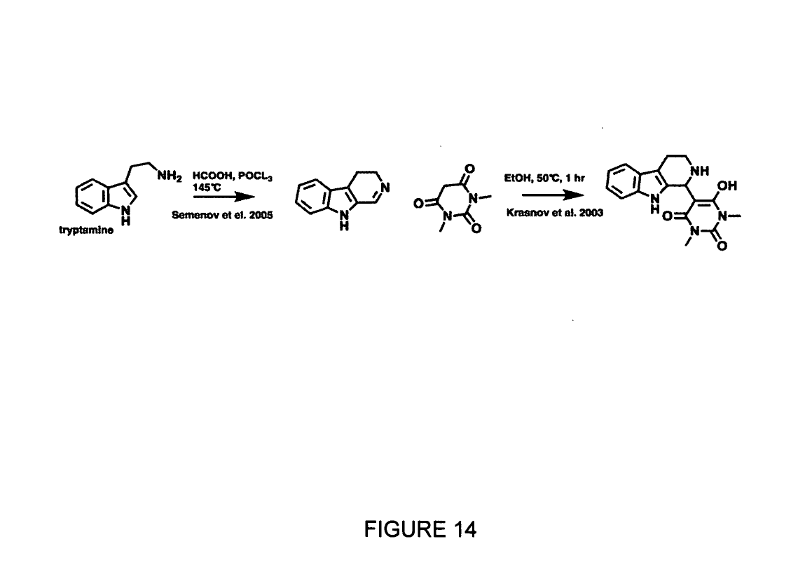

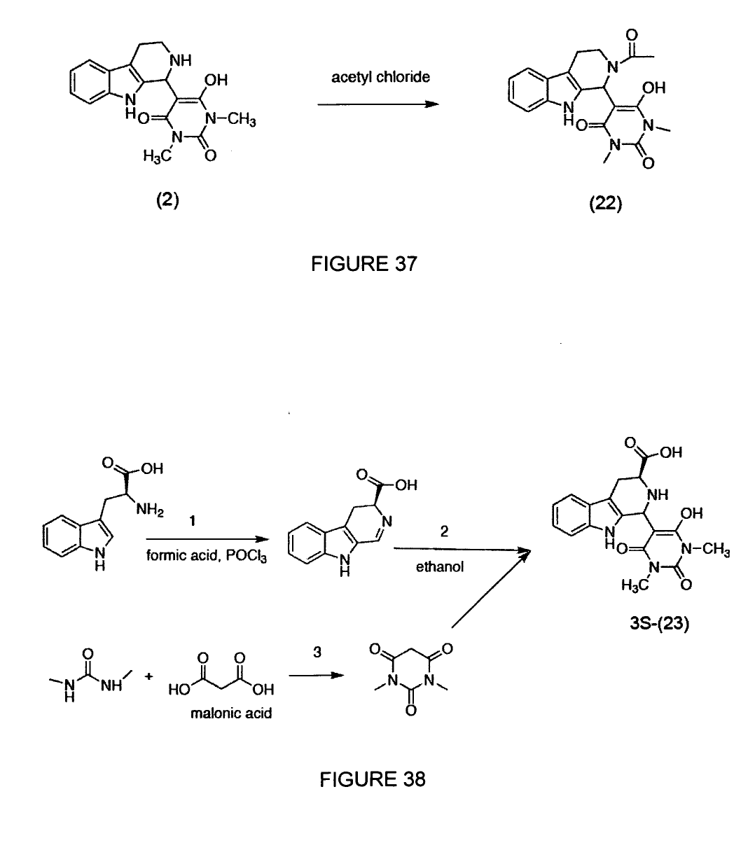

A representative synthetic scheme for making compound 22 is shown in FIG. 37. The starting compound, compound 2, may be made as set forth in FIG. 14 or as in Krasnov et al. (46).

### Example 21

### Synthetic Scheme for MEL24 Analogues, Compounds 23-32 and 34-36

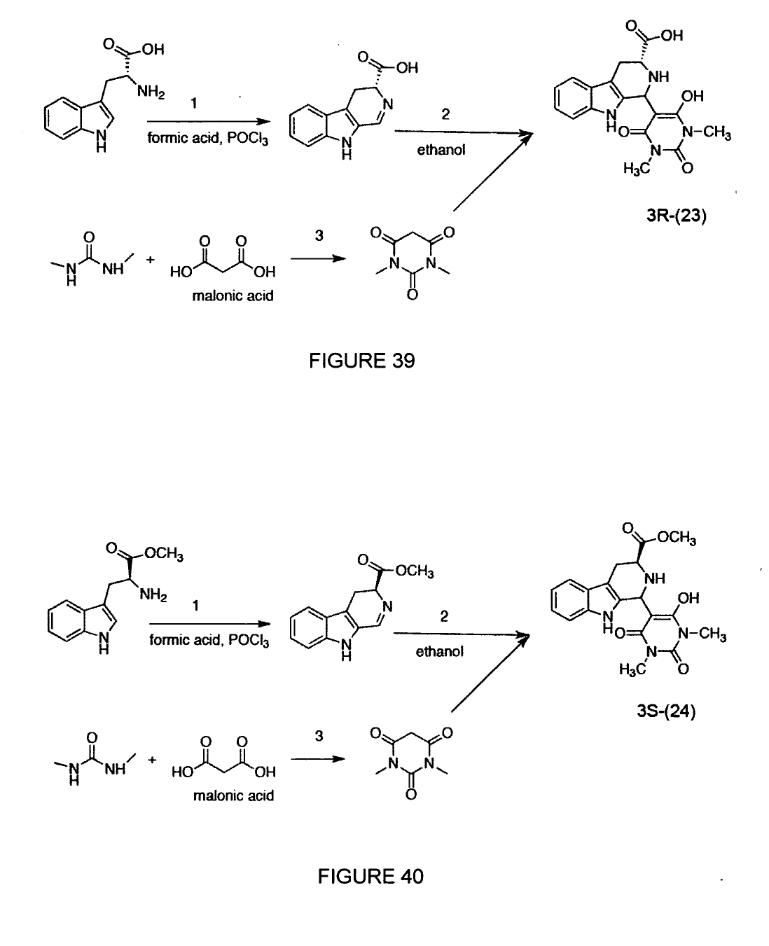

Representative synthetic schemes for making 3S and 3R forms of compound 23 are shown in FIGS. 38 and 39.

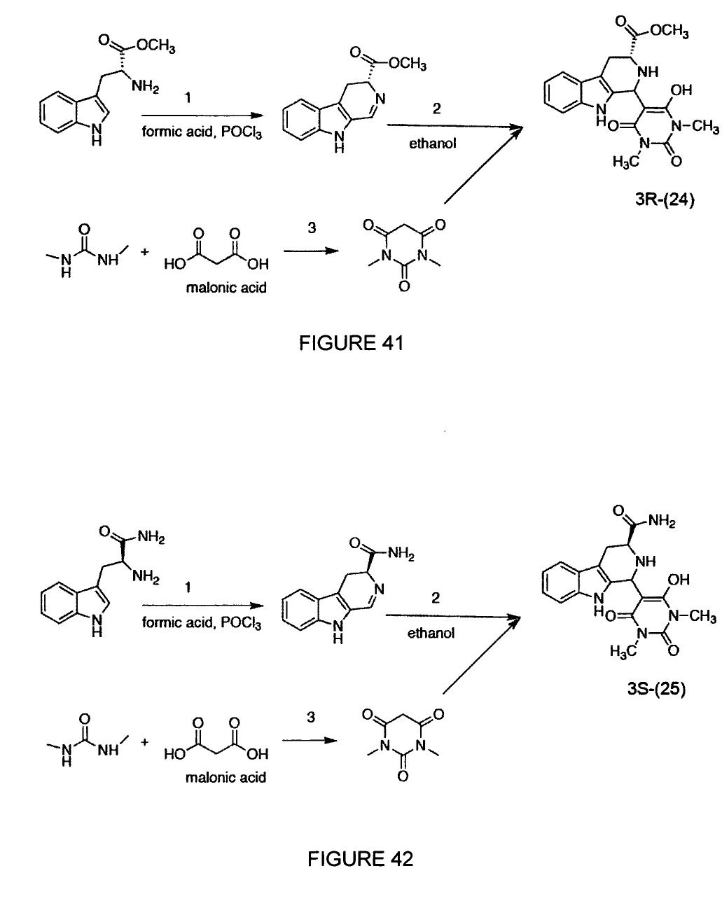

Representative synthetic schemes for making 3S and 3R forms of compound 24 are shown in FIGS. 40 and 41.

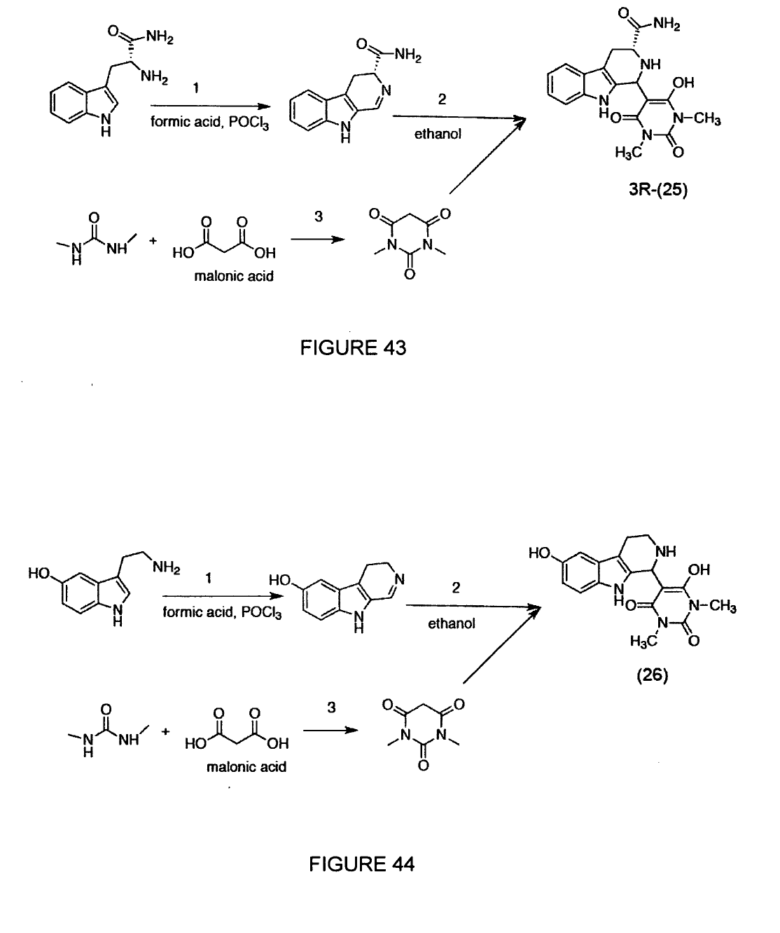

Representative synthetic schemes for making 3S and 3R forms of compound 25 are shown in FIGS. 42 and 43.

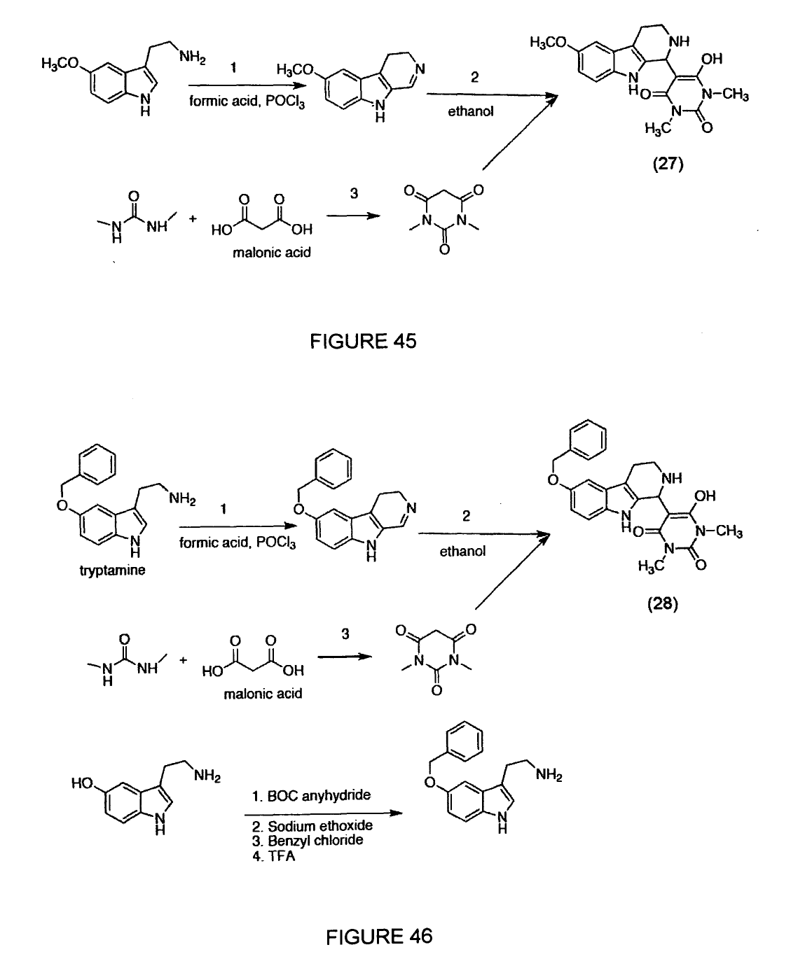

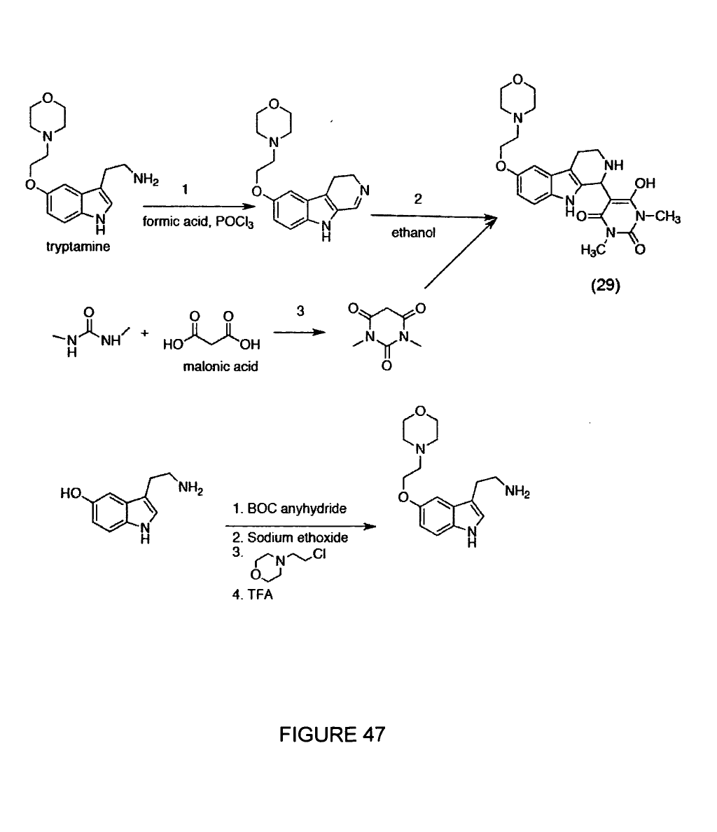

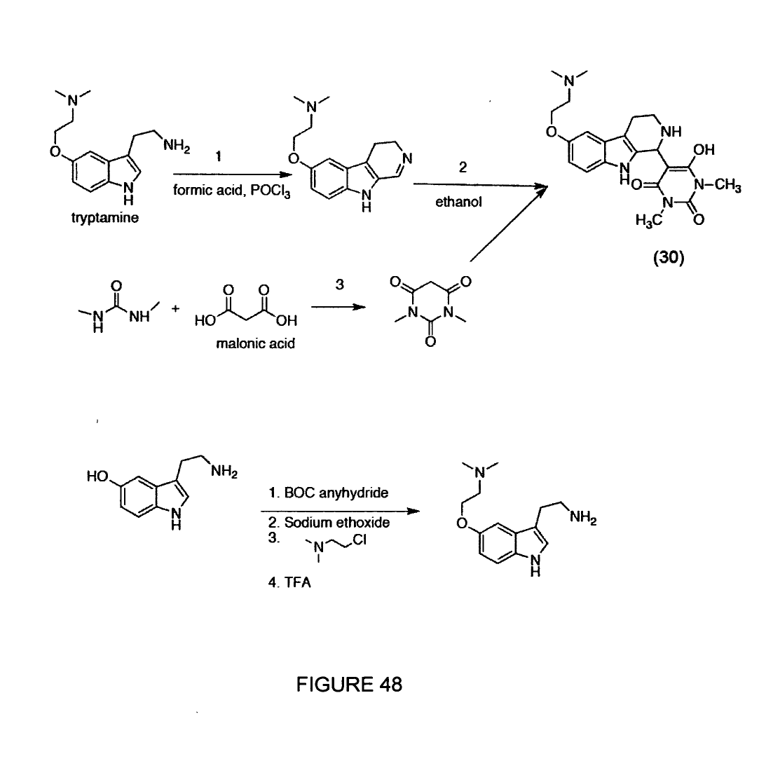

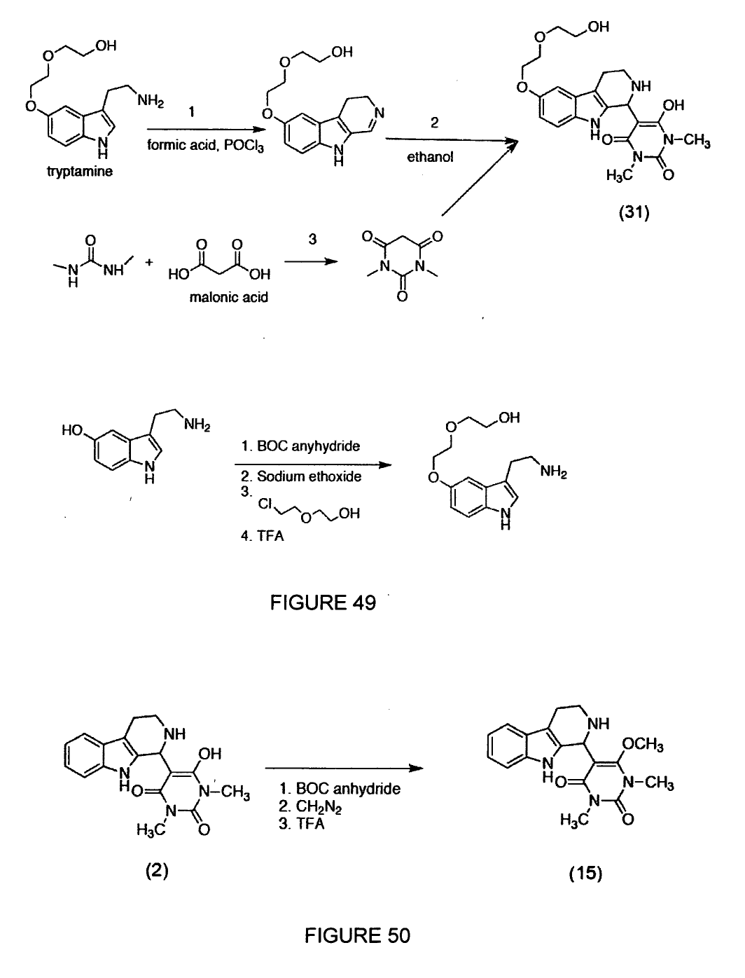

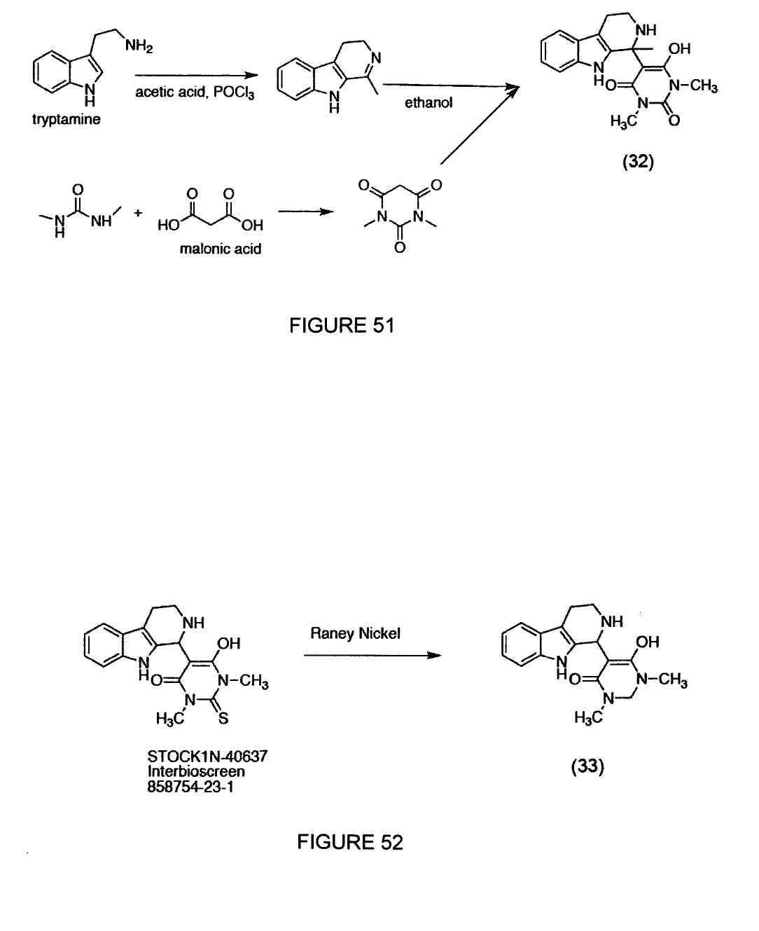

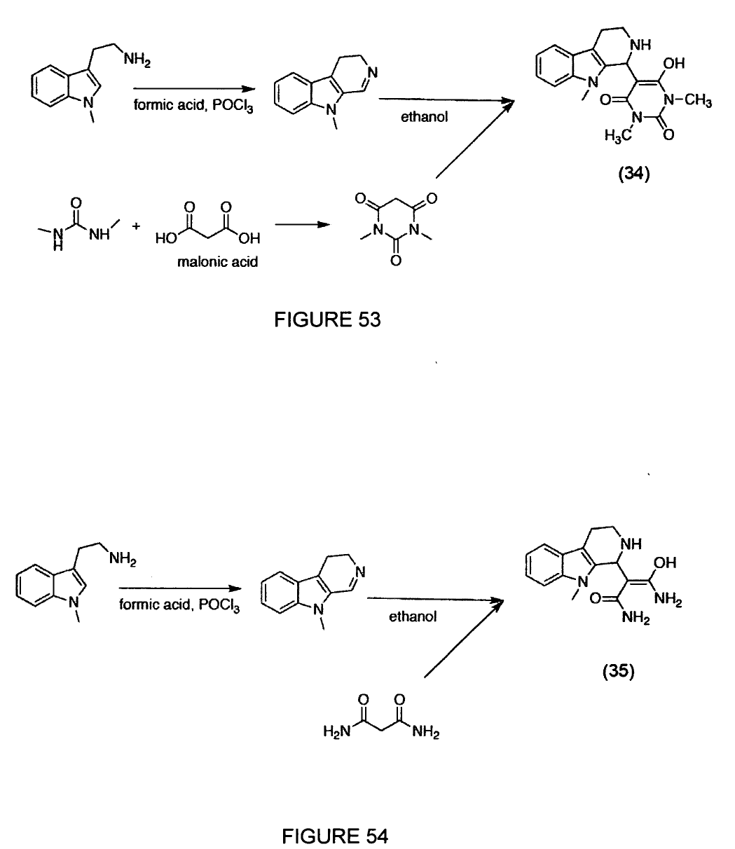

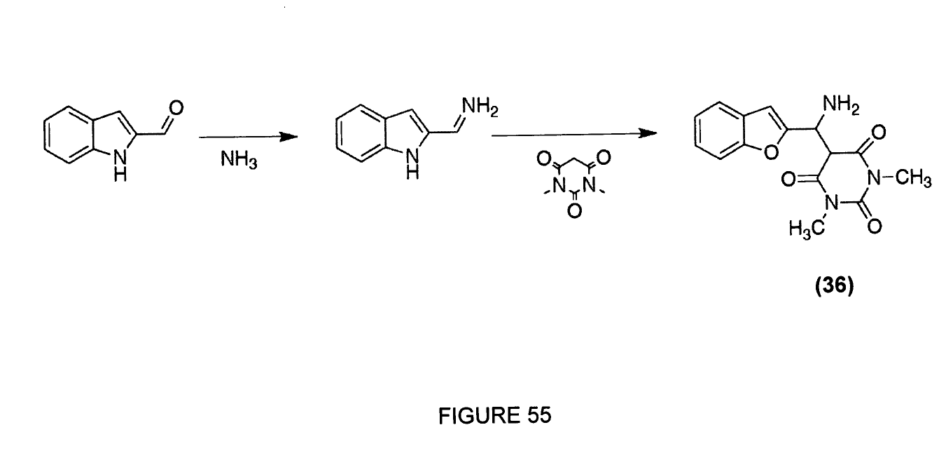

Representative synthetic schemes for making compounds 26-32, and 34-36 are shown in FIGS. 44-49, 51, 53-55, respectively.

### Example 22

### Alternative Synthetic Scheme for MEL24 Analogue, Compound 15

An alternative synthetic scheme for making compound 15 is shown in FIG. 50. The starting compound, compound 2, may be made as set forth in FIG. 14 or as in Krasnov et al. (46).

### Example 23

### Synthetic Scheme for MEL24 Analogue, Compound 33

A representative synthetic scheme for making compound 22 is shown in FIG. 37. The starting compound, STOCK1N-40637, may be obtained from Interbioscreen Ltd. (Moscow, Russia) or other commercial sources. The reaction may be carried out as set forth in Ramachary et al. (82).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### CITED DOCUMENTS

All documents cited herein, including those identified below, are incorporated by reference as if recited in full herein:
1. Glickman, M. H. and A. Ciechanover (2002). “The ubiquitin-proteasome proteolytic pathway: destruction for the sake of construction.” Physiol Rev 82(2): 373-428.
2. Jones, S, N., A. E. Roe, et al. (1995). “Rescue of embryonic lethality in Mdm2-deficient mice by absence of p53.” Nature 378(6553): 206-8.
3. Montes de Oca Luna, R., D. S. Wagner, et al. (1995). “Rescue of early embryonic lethality in mdm2-deficient mice by deletion of p53.” Nature 378(6553): 203-6.
4. Haupt, Y., R. Maya, et al. (1997). “Mdm2 promotes the rapid degradation of p53.” Nature 387(6630): 296-9.
5. Kubbutat, M. H., S, N. Jones, et al. (1997). “Regulation of p53 stability by Mdm2.” Nature 387(6630): 299-303.
6. M. H. Kubbutat, S, N.J., and K. H Vousden (1997). “Regulation of p53 stability by Mdm2.” Nature 387(6630): 299-303.
7. Momand J, Zambetti G P, et al. (1992). “The mdm-2 oncogene product forms a complex with the p53 protein and inhibits p53-mediated transactivation.” Cell. 69(7):1237-45.
8. Honda, R. and H. Yasuda (2000). “Activity of Mdm2, a ubiquitin ligase, toward p53 or itself is dependent on the RING finger domain of the ligase.” Oncogene 19(11): 1473-6.
9. Momand, J., D. Jung, et al. (1998). “The Mdm2 gene amplification database.” Nucleic Acids Res 26(15): 3453-9.
10. Martins, C. P., L. Brown-Swigart, et al. (2006). “Modeling the therapeutic efficacy of p53 restoration in tumors.” Cell 127(7): 1323-34.
11. Mendrysa, S. M., K. A. O'Leary, et al. (2006). “Tumor suppression and normal aging in mice with constitutively high p53 activity.” Genes Dev 20(1): 16-21.
12 Jones, S, N., A. R. Hancock, et al. (1998). “Overexpression of Mdm2 in mice reveals a p53-independent role for Mdm2 in tumorigenesis.” Proc Natl Acad Sci USA 95(26): 15608-12.
13. Vassilev, L. T., B. T. Vu, et al. (2004). “In vivo activation of the p53 pathway by small-molecule antagonists of Mdm2.” Science 303(5659): 844-8.
14. Lai, Z., T. Yang, et al. (2002). “Differentiation of Mdm2-mediated p53 ubiquitination and Hdm2 autoubiquitination activity by small molecular weight inhibitors.” Proc Natl Acad Sci USA 99(23): 14734-9.
15. Yang, Y., R. L. Ludwig, et al. (2005). “Small molecule inhibitors of HDM2 ubiquitin ligase activity stabilize and activate p53 in cells.” Cancer Cell 7(6): 547-59.
16. Murray, M. F., A. J. Jurewicz, et al. (2007). “A high-throughput screen measuring ubiquitination of p53 by human mdm2.” J Biomol Screen 12(8): 1050-8.
17. Clement, J. A., J. Kitagaki, et al. (2008). “Discovery of new pyridoacridine alkaloids from Lissoclinum cf. badium that inhibit the ubiquitin ligase activity of Hdm2 and stabilize p53.” Bioorg Med. Chem.
18. Kitagaki, J., K. K. Agama, et al. (2008). “Targeting tumor cells expressing p53 with a water-soluble inhibitor of Hdm2.” Mol Cancer Ther 7(8): 2445-54.
19. Sasiela, C. A., D. H. Stewart, et al. (2008). “Identification of inhibitors for Mdm2 ubiquitin ligase activity from natural product extracts by a novel high-throughput electrochemiluminescent screen.” J Biomol Screen 13(3): 229-37.
20. Wilkinson, K. D. Ubiquitination and deubiquitination: targeting of proteins for degradation by the proteasome. Semin Cell Dev Biol. 11: 141-8. (2000).
21. Schubert, U., Anton, L. C., Gibbs, J., Norbury, C. C., Yewdell, J. W. & Bennink, J. R. Rapid degradation of a large fraction of newly synthesized proteins by proteasomes. Nature. 404: 770-4 (2000).
22. Hershko, A. & Ciechanover, A. The ubiquitin system. Annu Rev Biochem. 67: 425-79. (1998).
23. Scheffner, M., Nuber, U. & Huibregtse, J. M. Protein ubiquitination involving an E1-E2-E3 enzyme ubiquitin thioester cascade. Nature. 373: 81-3. (1995).
24. Ciechanover, A. & Ben-Saadon, R. N-terminal ubiquitination: more protein substrates join in. Trends Cell Biol. 14: 103-6 (2004).
25. Fang, S. & Weissman, A. M. A field guide to ubiquitylation. Cell Mol Life Sci. 61: 1546-61 (2004).
26. Huibregtse, J. M., Scheffner, M., Beaudenon, S. & Howley, P. M. A family of proteins structurally and functionally related to the E6-AP ubiquitin-protein ligase. Proc Natl Aced Sci USA. 92: 2563-7. (1995).
27. Borden, K. L. & Freemont, P. S. The RING finger domain: a recent example of a sequence-structure family. Curr Opin Struct Biol. 6: 395-401 (1996).
28. Saurin, A. J., Borden, K. L., Boddy, M. N. & Freemont, P. S. Does this have a familiar RING? Trends Biochem Sci. 21: 208-14 (1996).
29. Iwakuma, T. & Lozano, G. Mdm2, an introduction. Mol Cancer Res. 1: 993-1000 (2003).
30. Yang, Y., Ludwig, R. L., Jensen, J. P., Pierre, S. A., Medaglia, M. V., Davydov, I. V., Safuran, Y. J., Oberoi, P., Kenten, J. H., Phillips, A. C., Weissman, A. M. & Vousden, K. H. Small molecule inhibitors of HDM2 ubiquitin ligase activity stabilize and activate p53 in cells. Cancer Cell. 7: 547-59 (2005).
31. Scherf, U., Ross, D. T., Waltham, M., Smith, L. H., Lee, J. K., Tanabe, L., Kohn, K. W., Reinhold, W. C., Myers, T. G., Andrews, D. T., Scudiero, D. A., Eisen, M. B., Sausville, E. A., Pommier, Y., Botstein, D., Brown, P. O. & Weinstein, J. N. A gene expression database for the molecular pharmacology of cancer. Nat. Genet. 24: 236-44 (2000).
32. Meulmeester, E., R. Frenk, et al. (2003). “Critical role for a central part of Mdm2 in the ubiquitylation of p53.” Mol Cell Biol 23(14): 4929-38.
33. Matheu A, Maraver A, et al., (2007). “Delayed ageing through damage protection by the Arf/p53 pathway.” Nature 448(7151):375-9.
34. Poyurovsky, M. V., Jacq, X., Ma, C., Karni-Schmidt, 0., Parker, P. J., Chalfie, M., Manley, J. L. & Prives, C. Nucleotide binding by the Mdm2 RING domain facilitates Arf-independent Mdm2 nucleolar localization. Mol. Cell. 12: 875-87 (2003).
35. Poyurovsky, M. V., Priest, C., Kentsis, A., Borden, K. L., Pan, Z. Q., Pavletich, N. & Prives, C. The Mdm2 RING domain C-terminus is required for supramolecular assembly and ubiquitin ligase activity. Embo J. 26: 90-101 (2007).
36. Karni-Schmidt, 0., Friedler, A., Zupnick, A., McKinney, K., Mattia, M., Beckerman, R., Bouvet, P., Sheetz, M., Fersht, A. & Prives, C. Energy-dependent nucleolar localization of p53 in vitro requires two discrete regions within the p53 carboxyl terminus. Oncogene. 26: 3878-91 (2007).
37. Shieh, S. Y., Ikeda, M., Taya, Y. & Prives, C. DNA damage-induced phosphorylation of p53 alleviates inhibition by Mdm2. Cell. 91: 325-34 (1997).
38. Wessel, D. & Flugge, U. I. A Method For The Quantitative Recovery Of Protein In Dilute-Solution In The Presence Of Detergents And Lipids. Analytical Biochemistry. 138: 141-143 (1984).
39. Unlu, M., Morgan, M. E. & Minden, J. S. Difference gel electrophoresis: A single gel method for detecting changes in protein extracts. Electrophoresis. 18: 2071-2077 (1997).
40. Blake, C. A., Brown, L. M., Duncan, M. W., Hunsucker, S. W. & Helmke, S. M. Estrogen regulation of the rat anterior pituitary gland proteome. Experimental Biology And Medicine. 230: 800-807 (2005).
41. Carr, S., Aebersold, R., Baldwin, M., Burlingame, A., Clauser, K. & Nesvizhskii, A. The need for guidelines in publication of peptide and protein identification data—Working group on publication guidelines for peptide and protein identification data. Molecular & Cellular Proteomics. 3: 531-533 (2004).
42. Zhang, T. & Prives, C. Cyclin a-CDK phosphorylation regulates Mdm2 protein interactions. J Biol. Chem. 276: 29702-10 (2001).
43. Okamoto, K., Li, H., Jensen, M. R., Zhang, T., Taya, Y., Thorgeirsson, S. S. & Prives, C. Cyclin G recruits PP2A to dephosphorylate Mdm2. Mol. Cell. 9: 761-71 (2002).
44. B. B. Semenov, K. A. Novikov, K. A. Krasnov and V. V. Kachala, Diastereocontrolled Synthesis of 6-Hydroxy-1,3-dimethyl-5-(R,R-4-phenyl-1,2,3,4-tetrahydro-1H-(3-carbolin-1-yl)-1H-pyrimidine-2,4-dione. Chemistry of Heterocyclic Compounds, 41 (5): 686-688 (2005).
45. Bruce G. Szczepankiewicz, Jeffrey J. Rohde, and Ravi Kurukulasuriya. Synthesis of Purines and Other Fused Imidazoles from Acyclic Amidines and Guanidines. Organic Letter, 7(9) 1833-1836 (2005).
46. Krasnov, K. A., Kartsev, V. G., and Yurova, M. N. Synthesis and Properties of N-Alkyl-6-hydroxy-5-(2,3,4,9-tetrahydro-1H-carbolin-1-yl)-1H-pyrimidine-2,4-diones and Their 2-Thioanalogs. Russian Journal of Organic Chemistry 39, 596-600. (2003).
47. Vandesompele, J. et al. Accurate normalization of real-time quantitative RT-PCR data by geometric averaging of multiple internal control genes. Genome Biol 3, RESEARCH0034 (2002).
48. Migliorini, D. et al. Mdm4 (Mdmx) regulates p53-induced growth arrest and neuronal cell death during early embryonic mouse development. Mol Cell Biol 22, 5527-38 (2002).
49. Finch, R. A. et al. mdmx is a negative regulator of p53 activity in vivo. Cancer Res 62, 3221-5 (2002).
50. Parant, J. et al. Rescue of embryonic lethality in Mdm-4-null mice by loss of Trp53 suggests a nonoverlapping pathway with MDM2 to regulate p53. Nat Genet. 29, 92-5 (2001).
51. Toledo, F. & Wahl, G. M. Regulating the p53 pathway: in vitro hypotheses, in vivo veritas. Nat Rev Cancer 6, 909-23 (2006).
52. Shvarts, A. et al. MDMX: a novel p53-binding protein with some functional properties of MDM2. EMBO J. 15, 5349-57 (1996).
53. Tanimura, S. et al. MDM2 interacts with MDMX through their RING finger domains. FEBS Lett 447, 5-9 (1999).
54. Linares, L. K., Hengstermann, A., Ciechanover, A., Muller, S. & Scheffner, M. HdmX stimulates Hdm2-mediated ubiquitination and degradation of p53. Proc Natl Acad Sci USA 100, 12009-14 (2003).
55. Gu, J. et al. Mutual dependence of MDM2 and MDMX in their functional inactivation of p53. J Biol Chem 277, 19251-4 (2002).
56. de Graaf, P. et al. Hdmx protein stability is regulated by the ubiquitin ligase activity of Mdm2. J Biol Chem 278, 38315-24 (2003).
57. Pan, Y. & Chen, J. MDM2 promotes ubiquitination and degradation of MDMX. Mol Cell Biol 23, 5113-21 (2003).
58. Kawai, H. et al. DNA damage-induced MDMX degradation is mediated by MDM2. J Biol Chem 278, 45946-53 (2003).
59. Karni-Schmidt, 0. et al. p53 is localized to a sub-nucleolar compartment after proteasomal inhibition in an energy-dependent manner. J Cell Sci 121, 4098-105 (2008).
60. Ko, L. J. et al. p53 is phosphorylated by CDK7-cyclin H in a p36MAT1-dependent manner. Mol Cell Biol 17, 7220-9 (1997).
61. Fridman, J. S. et al. Tumor promotion by Mdm2 splice variants unable to bind p53. Cancer Res 63, 5703-6 (2003).
62. Uchida, C. et al. Enhanced Mdm2 activity inhibits pRB function via ubiquitin-dependent degradation. EMBO J. 24, 160-9 (2005).
63. Sdek, P. et al. MDM2 promotes proteasome-dependent ubiquitin-independent degradation of retinoblastoma protein. Mol Cell 20, 699-708 (2005).
64. Zhang, Z. et al. MDM2 is a negative regulator of p21WAF1/CIP1, independent of p53. J Biol Chem 279, 16000-6 (2004).
65. Zhang, J. H., Chung, T. D. & Oldenburg, K. R. A Simple Statistical Parameter for Use in Evaluation and Validation of High Throughput Screening Assays. J Biomol Screen 4, 67-73 (1999).
66. Olson, D. C. et al. Identification and characterization of multiple mdm-2 proteins and mdm-2-p53 protein complexes. Oncogene 8, 2353-60 (1993).
67. Marine, J. C., Dyer, M. A. & Jochemsen, A. G. MDMX: from bench to bedside. J Cell Sci 120, 371-8 (2007).
68. Oren, M. Decision making by p53: life, death and cancer. Cell Death Differ 10, 431-42 (2003).
69. Scheffner, M., Huibregtse, J. M., Vierstra, R. D. & Howley, P. M. The HPV-16 E6 and E6-AP complex functions as a ubiquitin-protein ligase in the ubiquitination of p53. Cell 75, 495-505 (1993).
70. Tan, P. et al. Recruitment of a ROC1-CUL1 ubiquitin ligase by Skp1 and HOS to catalyze the ubiquitination of I kappa B alpha. Mol Cell 3, 527-33 (1999).
71. Chene, P. Inhibiting the p53-MDM2 interaction: an important target for cancer therapy. Nat Rev Cancer 3, 102-9 (2003).
72. Issaeva, N. et al. Small molecule RITA binds to p53, blocks p53-HDM-2 interaction and activates p53 function in tumors. Nat Med 10, 1321-8 (2004).
73. Fang, S., Jensen, J. P., Ludwig, R. L., Vousden, K. H. & Weissman, A. M. Mdm2 is a RING finger-dependent ubiquitin protein ligase for itself and p53. J Biol Chem 275, 8945-51 (2000).
74. Kawai, H., Lopez-Pajares, V., Kim, M. M., Wiederschain, D. & Yuan, Z. M. RING domain-mediated interaction is a requirement for MDM2's E3 ligase activity. Cancer Res 67, 6026-30 (2007).
75. Okamoto, K., Taya, Y. & Nakagama, H. Mdmx enhances p53 ubiquitination by altering the substrate preference of the Mdm2 ubiquitin ligase. FEBS Lett (2009).
76. Hashizume, R. et al. The RING heterodimer BRCA1-BARD1 is a ubiquitin ligase inactivated by a breast cancer-derived mutation. J Biol Chem 276, 14537-40 (2001).
77. Keith, C. T., Borisy, A. A. & Stockwell, B. R. Multicomponent therapeutics for networked systems. Nat Rev Drug Discov 4, 71-8 (2005).
78. Ma, J. et al. A second p53 binding site in the central domain of Mdm2 is essential for p53 ubiquitination. Biochemistry 45, 9238-45 (2006).
79. Andriamialisoa et al., “A new efficient total synthesis of vindorosine and vindoline,” J. Org. Chem., Vol. 50, No. 7, 1985, 961-967 (2005).
80. U.S. Pat. No. 6,458,474.
81. Reed, D. et al., “Identification and characterization of the first small-molecule inhibitor of MDMX.” J Biol. Chem., First Published on Jan. 15, 2010, doi: 10.1074/jbc.M109.056747.
82. Ramachary et al., “Multi-catalysis cascade reactions based on the methoxycarbonylketene platform: diversity-oriented synthesis of functionalized non-symmetrical malonates for agrochemicals and Pharmaceuticals.” Org. Biomol. Chem., Vol. 7, pp. 2053-2062 (2009).

What is claimed is:

1. A compound according to formula (I): 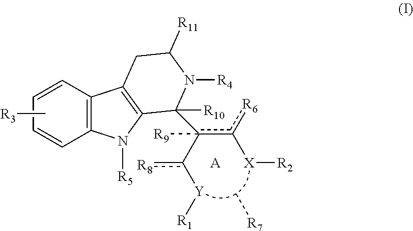
wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₅alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
or an enantiomer, optical isomer, diastereomer, N-oxide, crystalline form, hydrate, or pharmaceutically acceptable salt thereof,
with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
with the second proviso that (1) R₁ is not p-PhOCH₃, (2) R₄ is not methyl, and (3) R₃ is not methoxy;
and with the third proviso that the compound is not:

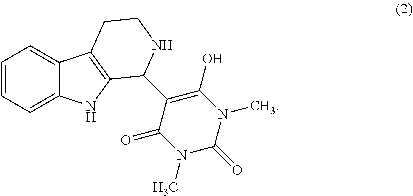

2. The compound according to claim 1, wherein A is a six-membered ring.

3. The compound according to claim 2, wherein R₆, R₇, and R₈ are not all oxo.

4. The compound according to claim 1, wherein R₁ and R₂ are independently selected from H, methyl, and phenyl.

5. The compound according to claim 1, wherein R₁ and R₂ are not both H.

6. The compound according to claim 1, wherein the compound is further not:

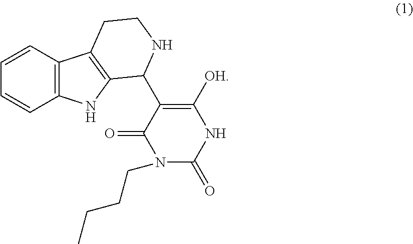

7. A compound according to claim 1 having formula (II): 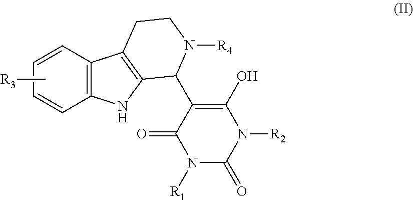
wherein:
R₁, R₂, R₃, and R₄ are independently selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

8. A compound according to claim 1 having the formula (III): 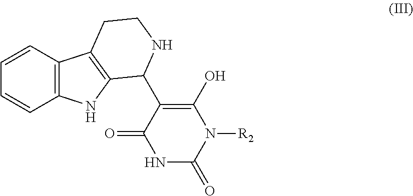
wherein
R₂ is selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

9. A compound according to claim 1 having formula (IV): 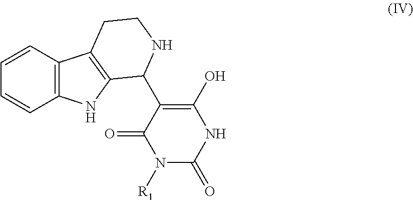
wherein
R₁ is selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

10. A compound according to claim 1 having the formula (V): 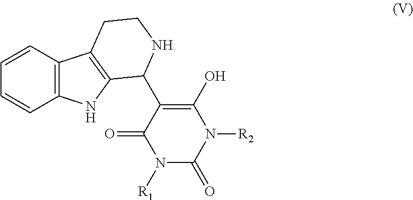
wherein
R₁ and R₂ are independently selected from the group consisting of H, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent; or
an enantiomer, optical isomer, diastereomer, N-oxide, crystalline form, hydrate, or pharmaceutically acceptable salt thereof.

11. A compound according to claim 1 having the formula (VI): 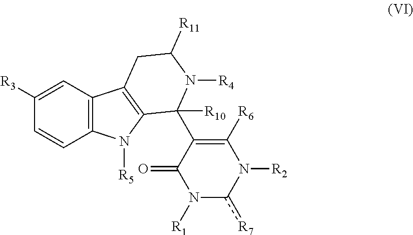
wherein:
R₁, R₂, R₃, R₄, R₅, R₆, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
a dashed line together with a solid line represent a single bond or a double bond;
wherein
if the bond linking a ring atom of A to R₇ is a double bond, R₇ is O; if the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
or an enantiomer, optical isomer, diastereomer, N-oxide, crystalline form, hydrate, or pharmaceutically acceptable salt thereof.

12. The compound according to claim 1, which is selected from the group consisting of: 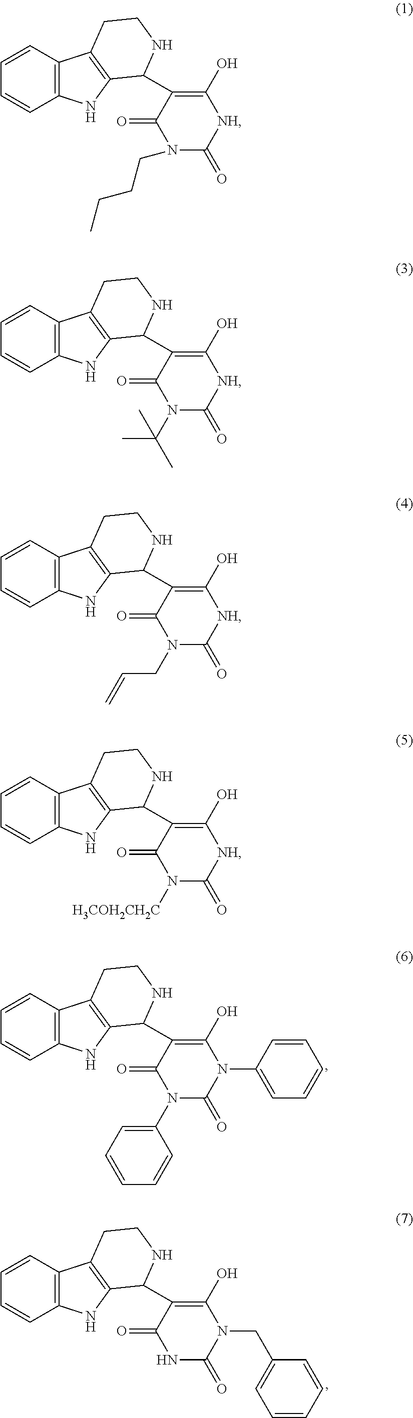 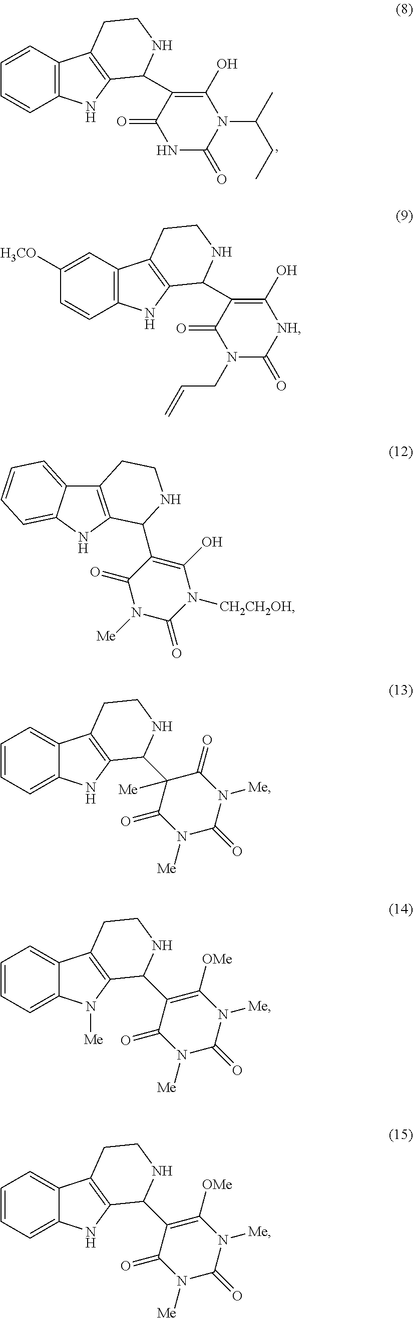 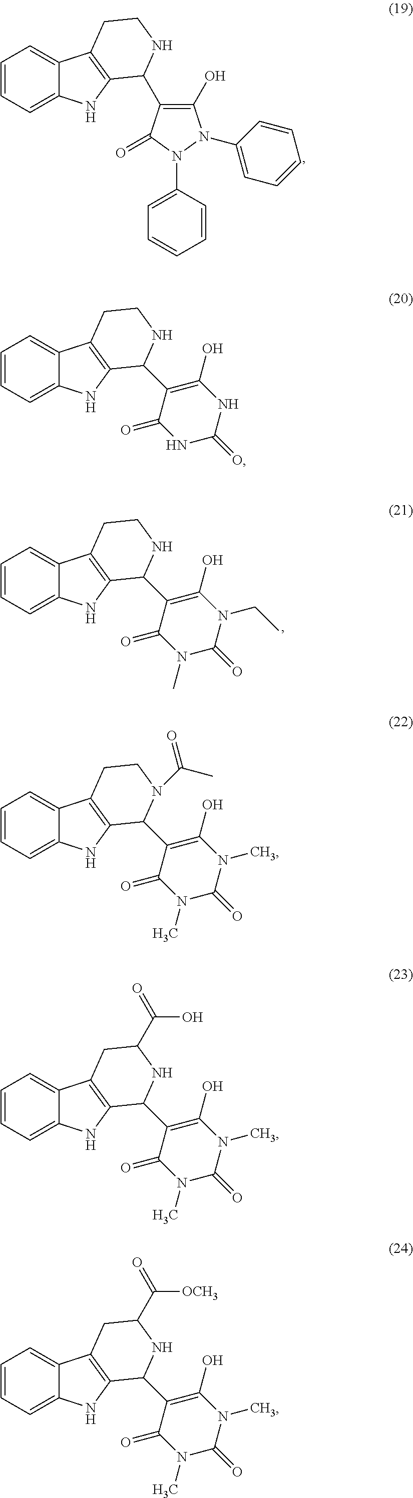 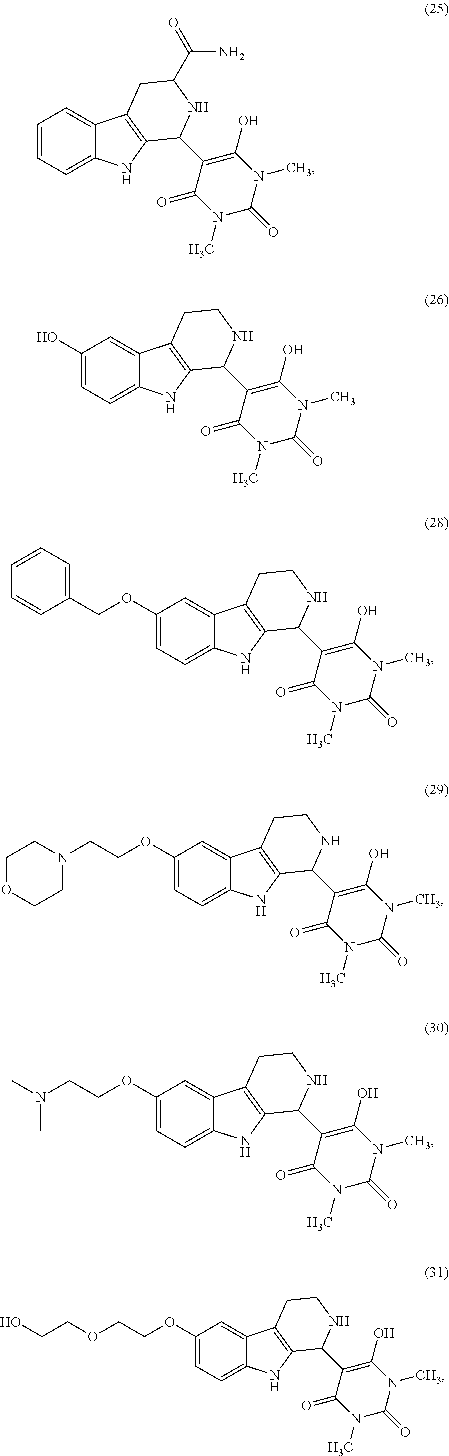 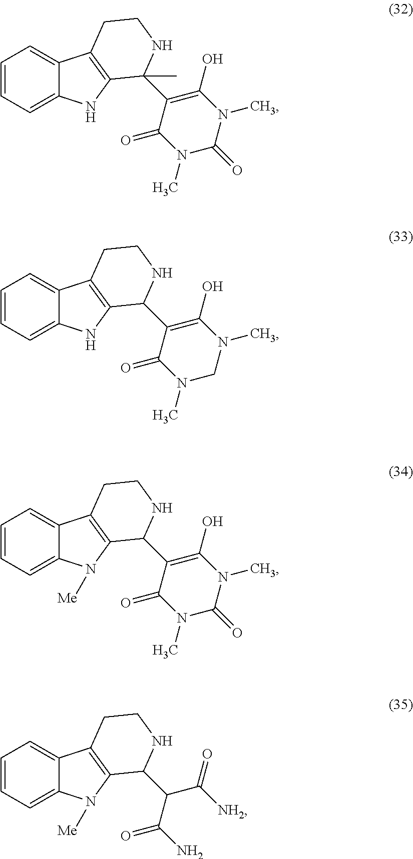
and an enantiomer, optical isomer, diastereomer, N-oxide, crystalline form, hydrate,
or pharmaceutically acceptable salt thereof.

13. A pharmaceutical composition comprising a pharmaceutically acceptable carrier and a compound according to formula (I): 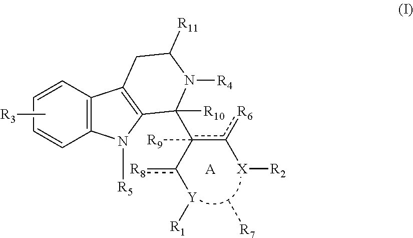
wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
or an enantiomer, optical isomer, diastereomer, N-oxide, crystalline form, hydrate, or pharmaceutically acceptable salt thereof,
with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
with the second proviso that (1) R₁ is not p-PhOCH₃, (2) R₄ is not methyl, and (3) R₃ is not methoxy;
and with the third proviso that the compound is not:

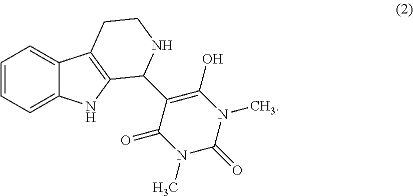

14. The pharmaceutical composition according to claim 13, wherein the compound is not:

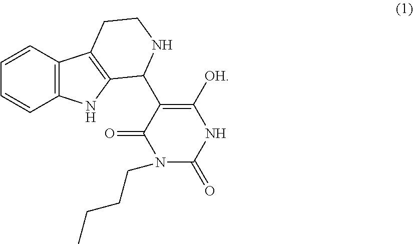

15. A pharmaceutical composition comprising a pharmaceutically acceptable carrier and a compound according to claim 13, which is selected from the group consisting of: 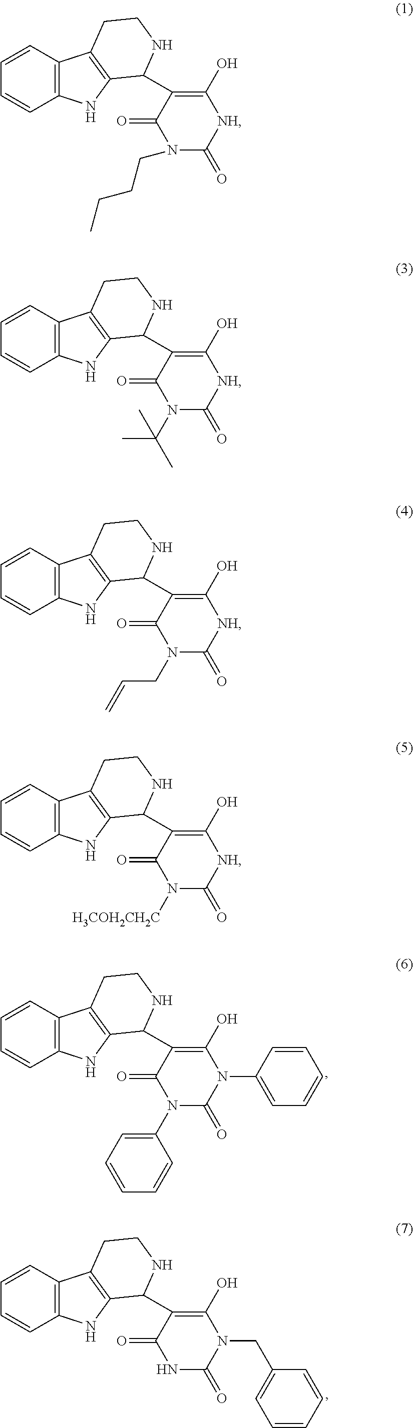 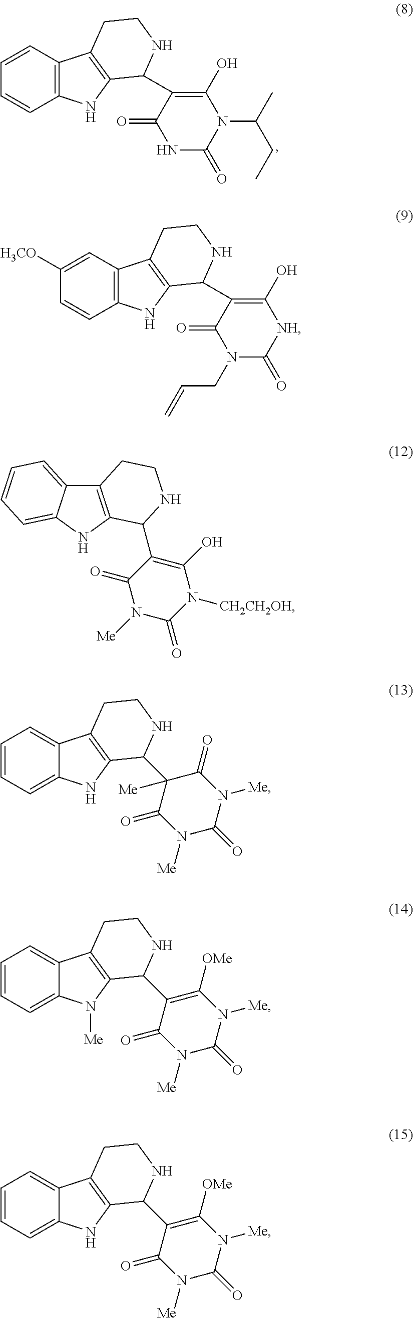 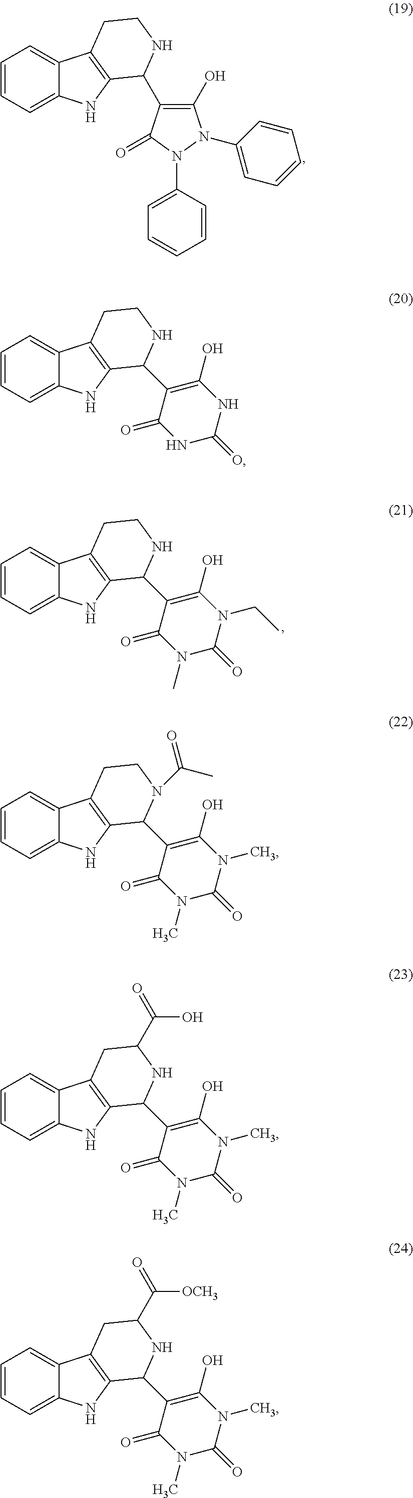 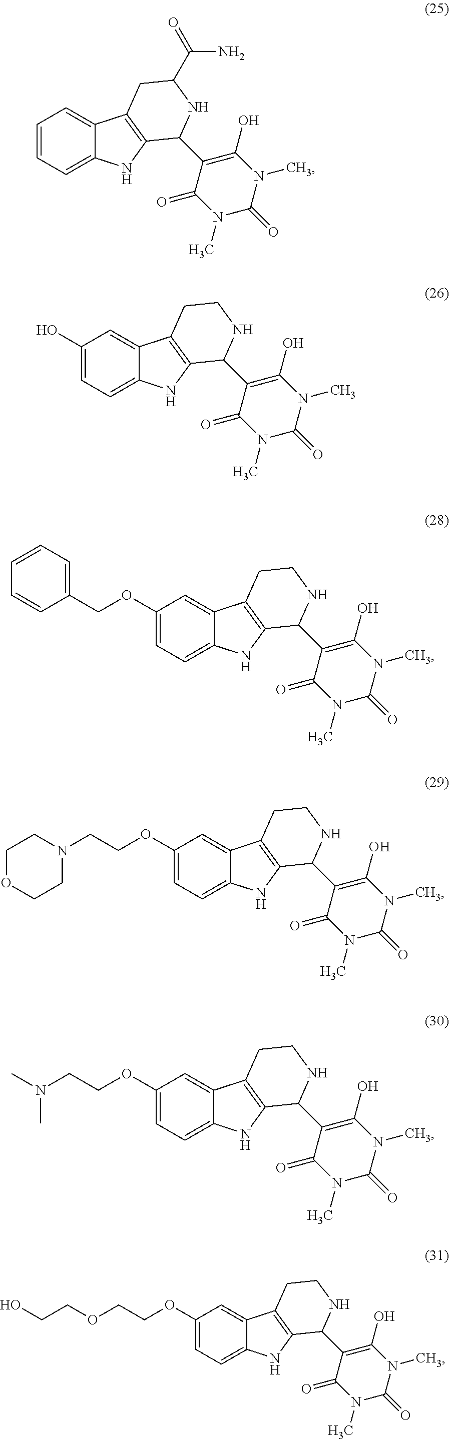 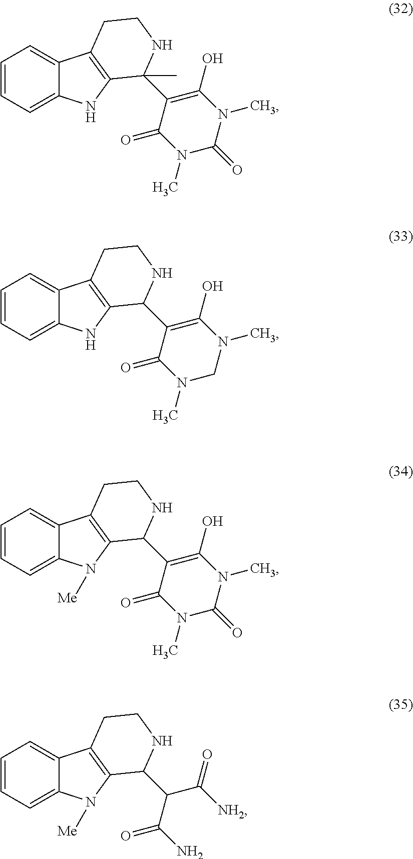
and an enantiomer, optical isomer, diastereomer, N-oxide, crystalline form, hydrate,
or pharmaceutically acceptable salt thereof.

16. A pharmaceutical composition comprising a compound according to claim 1, a pharmaceutically acceptable carrier, and at least one DNA damaging agent.

17. The pharmaceutical composition according to claim 16, wherein the DNA damaging agent is selected from the group consisting of actinomycin, amsacrine, anthracyclines, busulfan, cisplatin, cytoxan, epirubicin, hexamethylmelamineoxaliplatin, iphosphamide, mitoxantrone, taxotere, teniposide, triethylenethiophosphoramide, prednizone, dexamethasone, oxaliplatin, zoledronic acid, ibandronate, verapamil, podophyllotoxin, carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transpiatinum, 5-fluorouracil, vincristin, vinblastin, methotrexate, and combinations thereof.

18. A method for modulating murine double minute 2 protein (Mdm2) ligase activity comprising administering to a cell an amount of a compound according to formula (I): 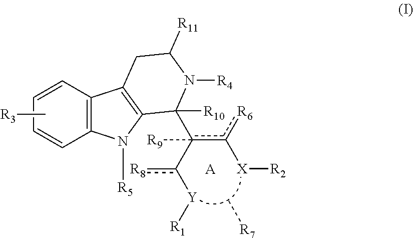
wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R^{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₈aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
or an enantiomer, optical isomer, diastereomer, N-oxide, crystalline form, hydrate, or pharmaceutically acceptable salt thereof,
with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
with the second proviso that (1) R₁ is not p-PhOCH₃, and (2) R₄ is not methyl;
which amount is effective to modulate Mdm2 ligase activity.

19. The method according to claim 18, wherein the Mdm2 ligase activity is Mdm2-MdmX hetero-complex E3 ligase activity.

20. A method for treating or ameliorating the effects of a cancerous disease comprising administering to a patient in need thereof an amount of a compound according to formula (I): 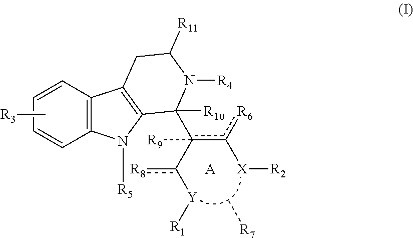
wherein:
A is a five-membered or six-membered ring, the ring optionally containing 1-3 heteroatoms, if X and Y are linked by one or more bonds;
R₁, R₂, R₃, R₄, R₅, R₉, R₁₀, and R₁₁ are independently selected from the group consisting of nothing, H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
Rₐ is selected from the group consisting of H, C₁₋₈alkyl, carbocycle, aryl, heteroaryl, heterocycle, alkylaryl, alkylheteroaryl, and alkylheterocycle, wherein each alkyl, carbocycle, aryl, heteroaryl, heterocycle, alkylaryl, alkylheteroaryl, and alkylheterocycle may be optionally substituted with at least one substituent;
R_{b} is selected from the group consisting of H, C₁₋₈alkyl, C₁₋₈alkenyl, C₁₋₈alkynyl, aryl, carbocycle, heteroaryl, heterocycle, alkylaryl, alkylheteroaryl, alkylheterocycle, and heteroaromatic, wherein each alkyl, alkenyl, alkynyl, aryl, carbocycle, heteroaryl, heterocycle, alkylaryl, alkylheteroaryl, alkylheterocycle, and heteroaromatic may be optionally substituted with at least one substituent;
X and Y are independently C or N, but X and Y cannot both be C if A is not a ring;
a single dashed line represents an optional bond, a single bond, or a double bond;
a dashed line together with a solid line represent a single bond or a double bond;
wherein
if the bond leading to R₆ is a double bond, R₆ is O; if the bond leading to R₆ is a single bond, R₆ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
if A is a six-membered ring, and the bond linking a ring atom of A to R₇ is a double bond, R₇ is O; if A is a six-membered ring, the bond linking a ring atom of A to R₇ is a single bond, R₇ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether; if A is not a six-membered ring, then R₇ is nothing;
if the bond leading to R₈ is a double bond, R₈ is O; if the bond leading to R₈ is a single bond, R₈ is selected from the group consisting of H, hydroxyl, C₁₋₈alkyl, C₁₋₈heteroalkyl, C₁₋₈alkenyl, C₁₋₈alkoxy, 3- to 8-membered carbocyclic or heterocyclic, aryl, heteroaryl, C₁₋₄aralkyl, residues of glycolic acid, ethylene glycol/propylene glycol copolymers, carboxylate, ester, amide, carbohydrate, amino acid, alditol, OC(Rₐ)₂COOH, SC(Rₐ)₂COOH, NHCHRₐCOOH, COR_{b}, CO₂R_{b}, sulfate, sulfonamide, sulfoxide, sulfonate, sulfone, thioalkyl, thioester, propylphthalimide, and thioether;
or an enantiomer, optical isomer, diastereomer, N-oxide, crystalline form, hydrate, or pharmaceutically acceptable salt thereof,
with the first proviso that (1) if A is a ring, then A is not an aryl; (2) if A is a ring, and R₇ is H or nothing, then at least one of R₁, R₂, R₆, R₈, and R₉ is not H, oxo, amino, aminoalkyl, acetamino, thio, or thioalkyl; or (3) if A is a ring, and R₇ is not H or nothing, then at least one of R₁, R₂, R₆, R₇, R₈, and R₉ is not H, oxo, amino, aminoalkyl, acetamino, thio, or thioalkyl;
with the second proviso that (1) R₁ is not p-PhOCH₃, and (2) R₄ is not methyl;
which amount is effective to treat or ameliorate the effects of the cancerous disease.

21. The method according to any one of claim 18 or 20, wherein the compound is selected from the group consisting of: 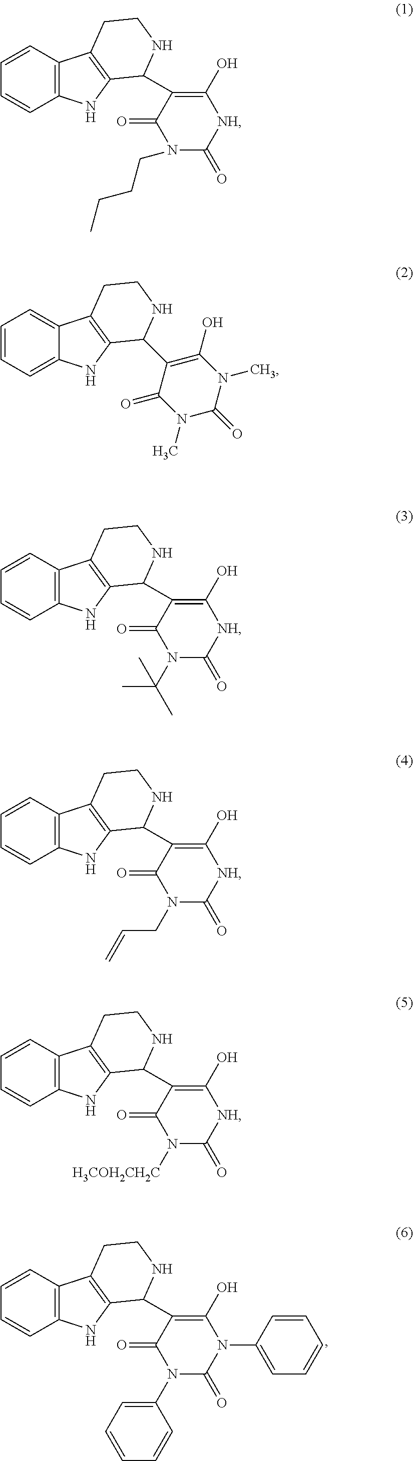 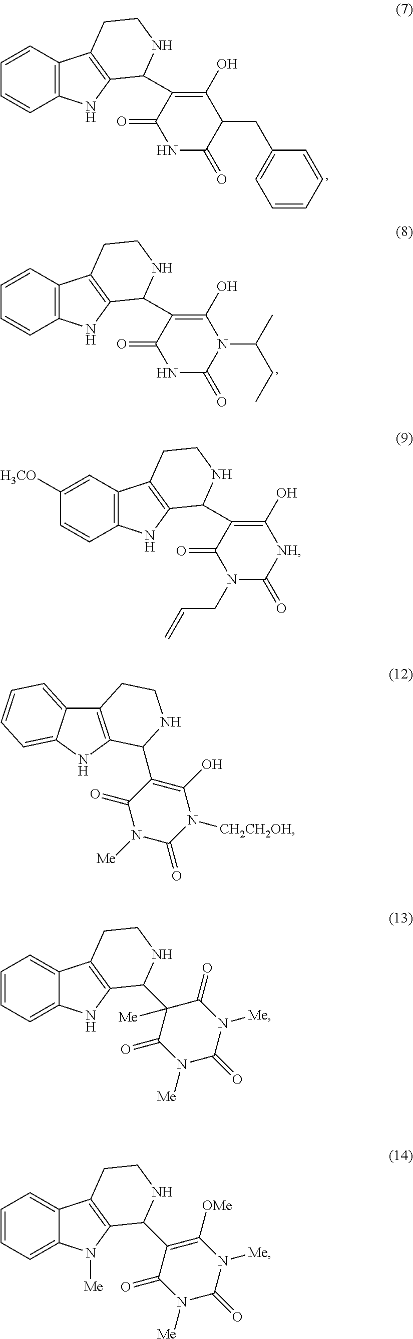 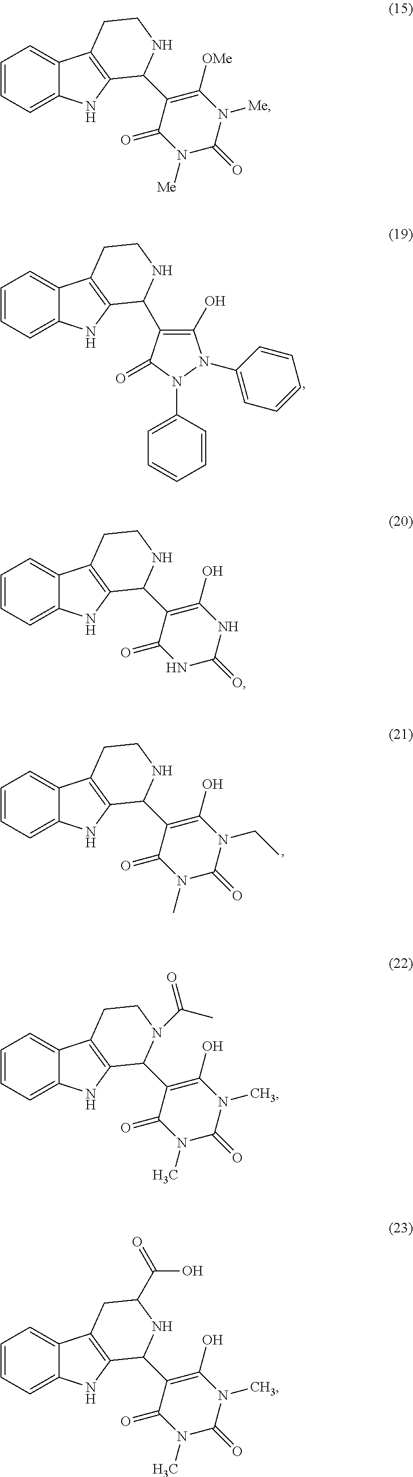 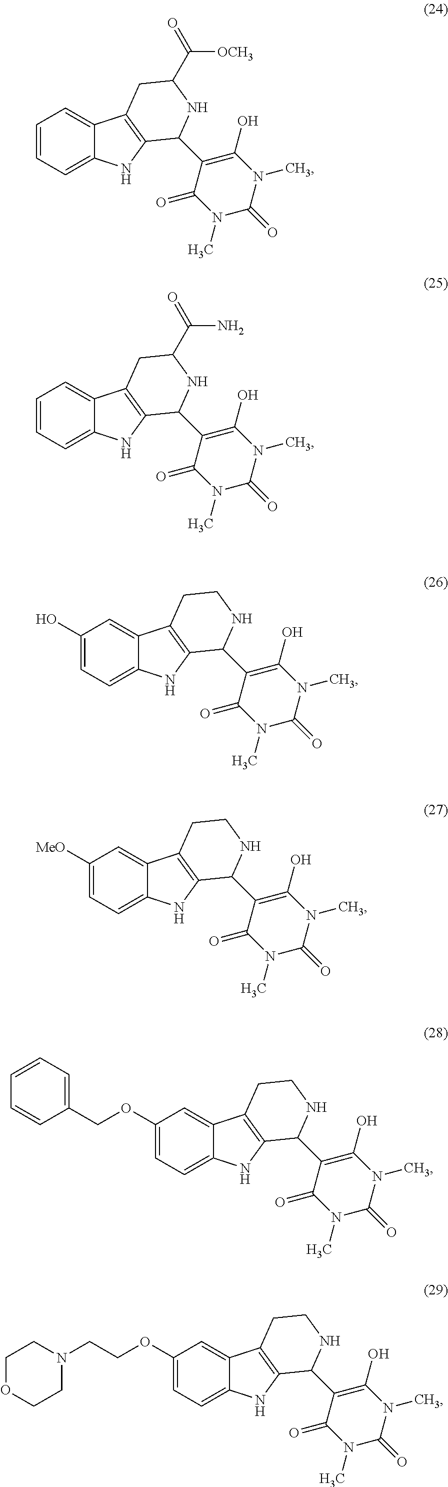 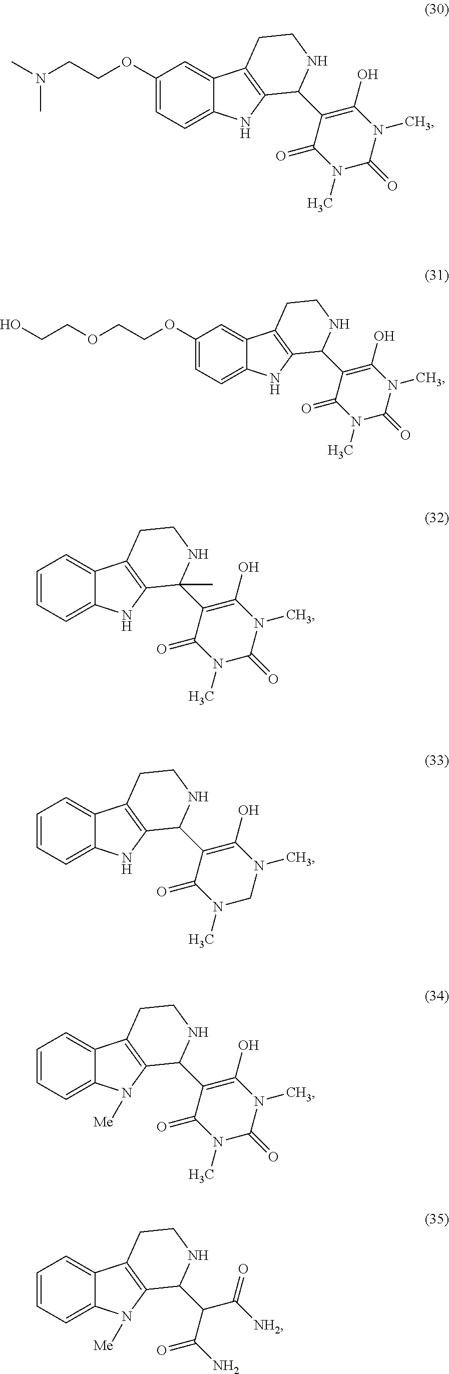
and an enantiomer, optical isomer, diastereomer, N-oxide, crystalline form, hydrate,
or pharmaceutically acceptable salt thereof.

22. The method according to any one of claim 18 or 20 further comprising administering a DNA damaging agent selected from the group consisting of actinomycin, amsacrine, anthracyclines, busulfan, cisplatin, cytoxan, epirubicin, hexamethylmelamineoxaliplatin, iphosphamide, mitoxantrone, taxotere, teniposide, triethylenethiophosphoramide, prednizone, dexamethasone, oxaliplatin, zoledronic acid, Ibandronate, verapamil, podophyllotoxin, carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transpiatinum, 5-fluorouracil, vincristin, vinblastin, methotrexate, and combinations thereof.

23. The method according to any one of claim 18 or 20, wherein the compound is further not:

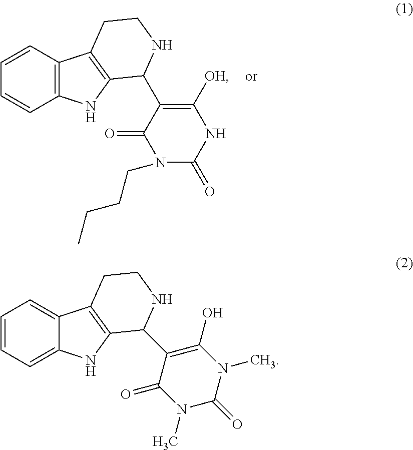

24. The method according to any one of claim 18 or 20, wherein the compound is further not:

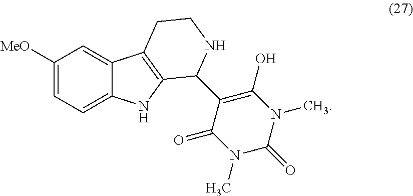

25. The method according to any one of claim 18 or 20, wherein A is a six-membered ring.

26. The method according to claim 25, wherein R₆, R₇, and R₈ of the compound having formula (I) are not all oxo.

27. The method according to claim 20, wherein R₁ and R₂ of the compound having formula (I) are independently selected from H, methyl, and phenyl.

28. The method according to claim 20, wherein R₁ and R₂ of the compound having formula (I) are not both H.

29. The method according to claim 20, wherein R₃ of the compound having formula (I) is not methoxy.

30. A compound having the structure of 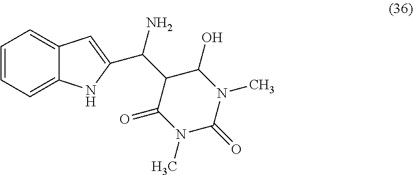
or an enantiomer, optical isomer, diastereomer, N-oxide, crystalline form, hydrate, or pharmaceutically acceptable salt thereof.

31. A pharmaceutical composition comprising the compound according to claim 30 and a pharmaceutically acceptable carrier.

32. A method for modulating murine double minute 2 protein (Mdm2) ligase activity comprising administering to a cell an amount of a compound according to claim 30, which amount is effective to modulate Mdm2 ligase activity.

33. A method for treating or ameliorating the effects of a cancerous disease comprising administering to a patient in need thereof an amount of a compound according to claim 30, which amount is effective to treat or ameliorate the effects of the cancerous disease.
